(19) United States
(12) Patent Application Publication
(10) Pub. No.: US 2026/0124173 A1
(43) Pub. Date: May 7, 2026

(54) THERAPEUTIC ALKALOID COMPOUNDS

(71) Applicant: Sensorium Therapeutics, Inc., Boston, MA (US)

(72) Inventors: Jacob M. Hooker, Winchester, MA (US); Michael S. Placzek, Tewksbury, MA (US)

(21) Appl. No.: 19/421,772

(22) Filed: Dec. 16, 2025

Related U.S. Application Data
(63) Continuation of application No. PCT/US2024/055181, filed on Nov. 8, 2024.
(60) Provisional application No. 63/548,083, filed on Nov. 10, 2023.
(60) Provisional application No. 63/651,580, filed on May 24, 2024.

(51) Int. Cl. A61K 31/404; C07D 209/08; C07D 405/04

(52) U.S. Cl. CPC A61K 31/404; C07D 209/08; C07D 405/04

(57) ABSTRACT

Disclosed are compounds useful for inhibiting SERT, and related methods of preparing and using these compounds.

### RELATED APPLICATIONS

This application claims the benefit of priority to U.S. Provisional patent application Ser. No. 63/548,083, filed Nov. 10, 2023; and U.S. Provisional patent application Ser. No. 63/651,580, filed May 24, 2024; the contents of each of which are hereby incorporated by reference in their entirety.

### TECHNICAL FIELD

The present disclosure relates to the field of medicine, including the discovery of alkaloid compounds useful for eliciting antidepressant and/or anxiolytic effects by inhibiting, in part, the serotonin transporter protein (5-HT).

### BACKGROUND

Serotonin (5-HT) is an essential neurotransmitter for the normal function of the central nervous system. This neurotransmission system in the brain controls various important behaviors, including sleep awake cycle, mood, temperature, appetite, etc. In addition, several commonly used anti-anxiety drugs.

Certain SERT inhibitors, including the selective serotonin transporter inhibitors, also called selective serotonin reuptake inhibitors (SSRIs), are used as therapeutic antidepressant drugs. They are believed to exert their effect by increasing extracellular 5-HT levels in the serotoninergic terminal fields such as the hippocampus and prefrontal cortex.

A fundamental evaluation in drug development is the assessment of absorption, distribution, metabolism, excretion, and pharmacokinetics (ADME/PK). One of the primary ADME screens that a novel chemical entity is subjected to is an in vitro metabolic stability screen. Drug stability upon exposure to human liver hepatocytes or microsomes is a common in vitro assay to approximate in vivo, liver-based drug metabolism.

There remains a need for therapeutic compounds that inhibit SERT with desired ADME and PK properties, such as extended half-life and metabolic stability, to provide adequate duration of action for therapeutic use.

### SUMMARY

Described herein are compounds of Formula (I), or a pharmaceutically acceptable salt thereof:

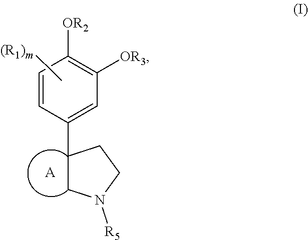

wherein
ring A is

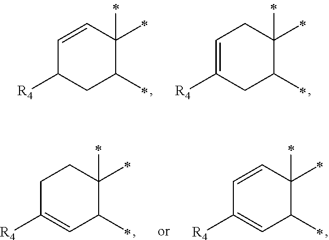

wherein * denotes the attachment points of ring A to the compound of formula (I), and wherein
each R¹ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O) NR^{a}R^{b}, —CN, nitro, or —P(O)OR^{a}OR^{b};
m is 0, 1, 2, or 3;
each of R² and R³ is independently H, alkyl, alkenyl, alkynyl, cycloalkyl, heterocycloalkyl, or aryl; wherein each hydrogen atom in alkyl cycloalkyl, alkenyl, alkynyl, and aryl is optionally substituted by halo, deuterium, cycloalkyl, aryl, or OR^{a}; or
R² and R³ together with the atoms to which they are attached combine to form heterocyclyl or heteroaryl, wherein each hydrogen atom in heterocyclyl and heteroaryl is optionally substituted by halo or OR^{a};
R^{a} and R^{b} are independently H, alkyl (e.g., methyl), alkenyl, cycloalkyl, heterocycloalkyl, aryl, or heteroaryl; or if an instance of R¹ is —NR^{a}R^{b}, then R^{a} and R^{b} may combine with the nitrogen atom to which they are attached to form heterocycloalkyl or heteroaryl;
R₄ is H, C₁₋₄ alkyl or C₁₋₆ haloalkyl; and
R₅ is C₁₋₄ alkyl.

In certain embodiments, the compound is a compound of Formula (I), wherein the compound is a compound of Formula (II-A), or a pharmaceutically acceptable salt thereof:

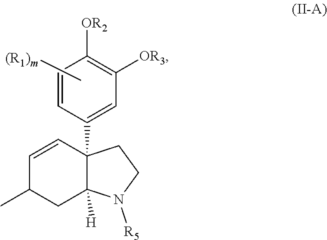

wherein R₁, m, R₂, R₃ and R₅ are as disclosed with respect to Formula (I) herein.

In certain embodiments, the compound is a compound of Formula (I), wherein the compound is a compound of Formula (II-B), or a pharmaceutically acceptable salt thereof:

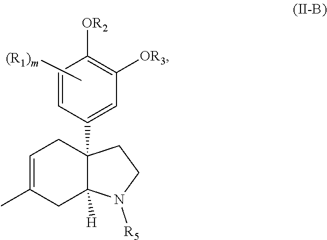

wherein R₁, m, R₂, R₃ and R₅ are as disclosed with respect to Formula (I) herein.

In certain embodiments, the compound is a compound of Formula (I), wherein the compound is a compound of Formula (II-C), or a pharmaceutically acceptable salt thereof:

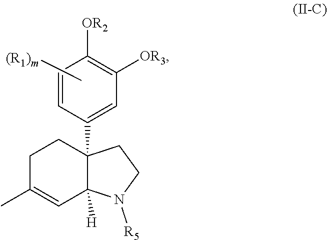

wherein R₁, m, R₂, R₃ and R₅ are as disclosed with respect to Formula (I) herein.

In certain embodiments, the compound is a compound of Formula (I), wherein the compound is a compound of Formula (II-D), or a pharmaceutically acceptable salt thereof:

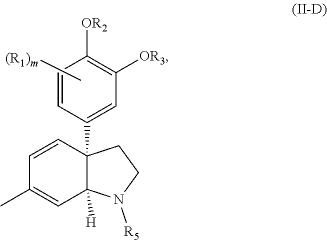

wherein R₁, m, R₂, R₃ and R₅ are as disclosed with respect to Formula (I) herein.

In certain embodiments, the compound is a compound of Formula (III), or a pharmaceutically acceptable salt thereof:

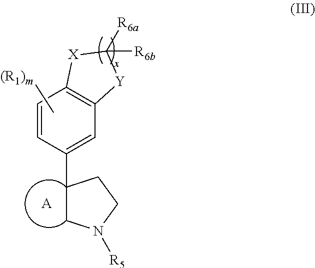

wherein Ring A, R₁, m, R^{a} and R₅ are as disclosed herein for Formula (I) or Formulae (II-A), (II-B), (II-C) and (II-D);
X and Y are each independently O, NR₇ or S;
x is 1 or 2;
each of R₆ₐ and R_{6b} is independently H, halo, C₁₋₄ alkyl, or C₁₋₄ haloalkyl; or

R₆ₐ and R_{6b} taken together with the atoms to which they are attached combine to form carbonyl (C═O), C₃₋₆ spirocyclic-cycloalkyl, or a 3-6 membered spirocyclic heterocyclyl comprising one or more O, N or S heteroatoms; wherein each hydrogen atom in cycloalkyl and heterocyclyl is optionally substituted by halo or OR^{a}; and
R₇ is H or C₁₋₄ alkyl.

In certain aspects, the invention relates to compounds of Formula (III), or a pharmaceutically acceptable salt thereof, wherein
ring A is

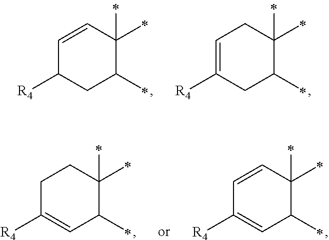

wherein * denotes the attachment points of ring A to the compound of formula (I), and wherein
each R₁ is independently deuterium, halo, C₁₋₄ alkyl, C₁₋₄ alkenyl, C₁₋₄ alkynyl, C₃₋₆ cycloalkyl, C₄₋₆ cycloalkenyl, 3 to 6-membered heterocyclyl, 5-10 membered aryl, 5-10 membered heteroaryl, —OR^{a}, —NR^{a}R^{b}, —CHO, —C(O)R^{a}, —CO₂R^{a}, —C(O) NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O) NR^{a}R^{b}, —CN, nitro, or —P(O)OR^{a}OR^{b};
m is 0, 1, 2, or 3;
R₄ is H, C₁₋₄ alkyl or C₁₋₆ haloalkyl; and
R₅ is C₁₋₄ alkyl optionally substituted with C₃₋₆ cycloalkyl, 3-6 membered heterocycle, phenyl or 5-6 membered heteroaryl, wherein the alkyl, cycloalkyl, heterocycle, aryl, or heteroaryl are each independently optionally substituted with one or more deuterium, C₁₋₄ alkoxy, C₁₋₄ haloalkyl, halo, cyano, amino, carboxy, acetyl, or amide;
each of R₆ₐ and R_{6b} is independently H, halo, or optionally substituted alkyl, wherein each hydrogen atom in the alkyl is optionally substituted by halo, deuterium, cycloalkyl, or OR^{a}; or
R₆ₐ and R_{6b} taken together with the atoms to which they are attached combine to form carbonyl (C═O), C₃₋₆ spirocyclic-cycloalkyl, or a 3-6 membered spirocyclic heterocyclyl comprising one or more O, N or S heteroatoms; wherein each hydrogen atom in cycloalkyl and heterocyclyl is optionally substituted by halo or OR^{a};
R₇ is H or C₁₋₄ alkyl; and
R^{a} and R^{b} are independently H, C₁₋₄ alkyl (e.g., methyl), or C₁₋₄ alkoxy.

In certain aspects, the invention relates to compounds of Formula (IV), or a pharmaceutically acceptable salt thereof:

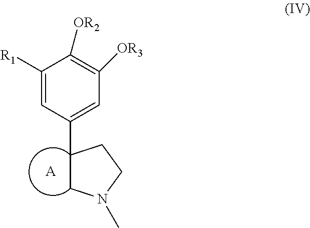

wherein
ring A is

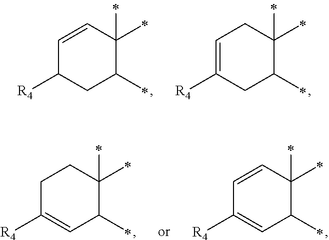

wherein * denotes the attachment points of ring A to the compound of formula (I), and wherein
each R¹ is independently halo, C₁₋₄ alkyl, C₁₋₄ haloalkyl, methoxy, acetyl, cyano, C₂₋₄ alkenyl, C₂₋₄ alkynyl, amino, amido or carboxyl;

each of R₂ and R₃ is independently H, or C₁₋₄ alkyl optionally substituted with one or more halo, C₃₋₆ cycloalkyl, alkenyl or alkynyl; or

R₂ and R₃ together with the atoms to which they are attached combine to form-(CR₆ₐR_{6b})ₓ—;
x is 1 or 2;
R₄ is methyl;
each of R₆ₐ and R_{6b} is independently H, C₁₋₄ alkyl, or C₁₋₄ haloalkyl; or
R₆ₐ and R_{6b} taken together with the atoms to which they are attached combine to form carbonyl (C═O), C₃₋₆ spirocyclic-cycloalkyl, or a 3-6 membered spirocyclic heterocyclyl comprising one or more O, N or S heteroatoms; wherein each hydrogen atom in cycloalkyl and heterocyclyl is optionally substituted by halo or OR^{a}; and
R^{a} is H, C₁₋₄ alkyl (e.g., methyl), C₁₋₄ haloalkyl or C₁₋₄ alkoxy.

In some embodiments, pharmaceutical compositions are also provided, including compositions comprising a compound of formula (I), including compounds of formulae (II-A), (II-B), (II-C) and (II-D), Formula (III) or Formula (IV), or a pharmaceutically acceptable salt thereof, and a pharmaceutically acceptable excipient.

In certain embodiments, the present disclosure provides a method of treating a central nervous condition, comprising administering to a subject in need thereof an effective amount of a compound of the present disclosure.

In certain embodiments, the present disclosure provides a method of treating a condition by administering a SERT inhibitor, comprising administering to a subject in need thereof an effective amount a compound of the present disclosure.

Numerous embodiments are further provided that can be applied to any aspect of the present invention described herein.

### DETAILED DESCRIPTION

The present invention is based, at least in part, on analogs of mesembrine and mesembrenone. Although (−) mesembrine is bioactive with certain desirable pharmacologic effects, certain other properties are less than ideal for use as a therapeutic. For example, the pharmacokinetics described for (−) mesembrine show rapid metabolism and excretion, with an undesirably low half-life in plasma of less than 2 hours. To take advantage of the desirable properties of mesembrine and mesembrenone, compounds have been developed and described here.

### Compounds of the Invention

In certain aspects, the invention relates to compounds of Formula (I), or a pharmaceutically acceptable salt thereof:

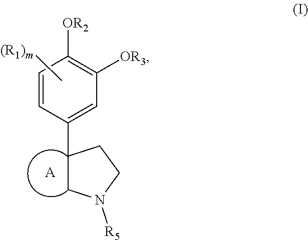

wherein
ring A is

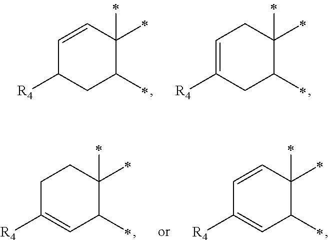

wherein * denotes the attachment points of ring A to the compound of formula (I), and wherein
each R₁ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O) NR^{a}R^{b}, —CN, nitro, or —P(O)OR^{a}OR^{b};
m is 0, 1, 2, or 3;
each of R₂ and R₃ is independently H, alkyl, alkenyl, alkynyl, cycloalkyl, heterocycloalkyl, or aryl; wherein each hydrogen atom in alkyl cycloalkyl, alkenyl, alkynyl, and aryl is optionally substituted by halo, deuterium, cycloalkyl, aryl, or OR^{a}; or
R² and R³ together with the atoms to which they are attached combine to form heterocyclyl or heteroaryl, wherein each hydrogen atom in heterocyclyl and heteroaryl is optionally substituted by halo or OR^{a};
R^{a} and R^{b} are independently H, alkyl (e.g., methyl), alkenyl, cycloalkyl, heterocycloalkyl, aryl, or heteroaryl; or if an instance of R₁ is —NR^{a}R^{b}, then R^{a} and R^{b} may combine with the nitrogen atom to which they are attached to form heterocycloalkyl or heteroaryl;
R₄ is H, C₁₋₄ alkyl or C₁₋₆ haloalkyl; and
R₅ is C₁₋₄ alkyl optionally substituted with C₃₋₆ cycloalkyl, 3-6 membered heterocycle, phenyl or 5-6 membered heteroaryl, wherein the alkyl, cycloalkyl, heterocycle aryl or heteroaryl are each independently optionally substituted with one or more deuterium, C₁₋₄ alkoxy, C₁₋₄ haloalkyl, halo, cyano, amino, carboxy, acetyl, or amide.

In some embodiments, a compound of Formula (I) is also a compound of formulae (II-A), (II-B), (II-C) or (II-D). In certain embodiments, the compound is a compound of Formula (I), wherein the compound is a compound of Formula (II-A), or a pharmaceutically acceptable salt thereof:

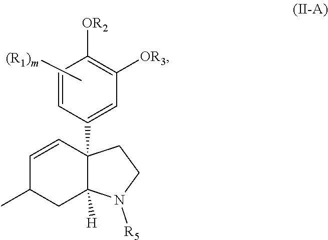

wherein R₁, m R₂, R₃ and R₅ are as defined with respect to Formula (I) herein.

In certain embodiments, the compound is a compound of Formula (I), wherein the compound is a compound of Formula (II-B), or a pharmaceutically acceptable salt thereof:

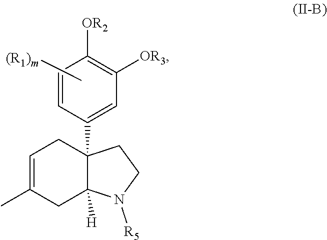

wherein R₁, m R₂, R₃ and R₅ are as defined with respect to Formula (I) herein.

In certain embodiments, the compound is a compound of Formula (I), wherein the compound is a compound of Formula (II-C), or a pharmaceutically acceptable salt thereof:

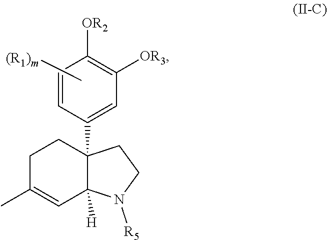

wherein R₁, m R₂, R₃ and R₅ are as defined with respect to Formula (I) herein.

In certain embodiments, the compound is a compound of Formula (I), wherein the compound is a compound of Formula (II-D), or a pharmaceutically acceptable salt thereof:

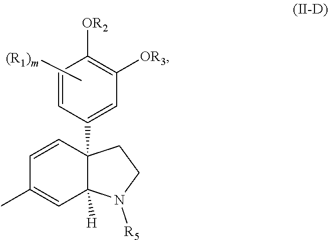

wherein R₁, m R₂, R₃ and R₅ are as defined with respect to Formula (I) herein.

In some embodiments, pharmaceutical compositions are also provided, including compositions comprising a compound of formula (I), including compounds of formulae (II-A), (II-B), (II-C) and (II-D), or a pharmaceutically acceptable salt thereof, and a pharmaceutically acceptable excipient.

In certain aspects, the invention relates to compounds of Formula (III), or a pharmaceutically acceptable salt thereof:

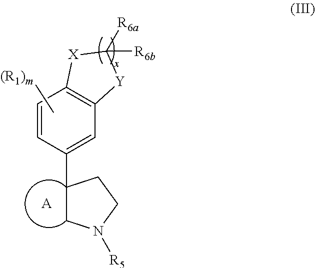

wherein Ring A, R₁, m, and R₅ are as disclosed herein for Formula (I) or Formula (II-A), (II-B), (II-C) and (II-D);
X and Y are each independently O, NR₇ or S;
x is 1 or 2;
each of R₆ₐ and R_{6b} is independently H, C₁₋₄ alkyl, or C₁₋₄ haloalkyl; or
R₆ₐ and R_{6b} taken together with the atoms to which they are attached combine to form carbonyl (C═O), C₃₋₆ spirocyclic-cycloalkyl, or a 3-6 membered spirocyclic heterocyclyl comprising one or more O, N or S heteroatoms; wherein each hydrogen atom in cycloalkyl and heterocyclyl is optionally substituted by halo or OR^{a};
R^{a} is H, C₁₋₄ alkyl (e.g., methyl), C₁₋₄ haloalkyl or C₁₋₄ alkoxy; and
R₇ is H or C₁₋₄ alkyl.

In certain aspects, the invention relates to compounds of Formula (III), or a pharmaceutically acceptable salt thereof, wherein
ring A is

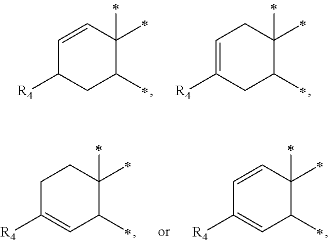

wherein * denotes the attachment points of ring A to the compound of formula (III), and wherein
each R₁ is independently deuterium, halo, C₁₋₄ alkyl, C₁₋₄ alkenyl, C₁₋₄ alkynyl, C₃₋₆ cycloalkyl, C₄₋₆ cycloalkenyl, 3 to 6-membered heterocyclyl, 5-10 membered aryl, 5-10 membered heteroaryl, —OR^{a}, —NR^{a}R^{b}, —CHO, —C(O)R^{a}, —CO₂R^{a}, —C(O) NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O) NR^{a}R^{b}, —CN, nitro, or —P(O)OR^{a}OR^{b};
m is 0, 1, 2, or 3;
R^{a} and R^{b} are independently H, or C₁₋₄ alkyl (e.g., methyl);
R₄ is methyl;
R₅ is C₁₋₄ alkyl;
each of R₆ₐ and R_{6b} is independently H or optionally substituted C₁₋₄ alkyl, wherein each hydrogen atom in the alkyl is optionally substituted by halo, C₃₋₆ cycloalkyl, or OR^{a}; or
R^{6a} and R^{6b} taken together with the atoms to which they are attached combine to form carbonyl (C═O), C₃₋₆ spirocyclic-cycloalkyl, or a 3-6 membered spirocyclic heterocyclyl comprising one or more O, N or S heteroatoms; wherein each hydrogen atom in cycloalkyl and heterocyclyl is optionally substituted by halo or OR^{a}, and
R₇ is H or C₁₋₄ alkyl.

In certain aspects, the invention relates to compounds of Formula (III), or a pharmaceutically acceptable salt thereof, wherein
ring A is

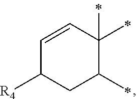

wherein * denotes the attachment points of ring A to the compound of formula (III), and wherein
each R₁ is independently halo, C₁₋₄ alkyl, C₁₋₄ alkenyl, C₁₋₄ alkynyl, C₃₋₆ cycloalkyl, C₁₋₄ haloalkyl or C₁₋₄ alkoxy;
m is 0, 1, 2, or 3;
R^{a} and R^{b} are independently H, or C₁₋₄ alkyl (e.g., methyl);
R₄ is methyl; and
R₅ is methyl;
each of R₆ₐ and R_{6b} is independently H or optionally substituted alkyl, wherein each hydrogen atom in the alkyl is optionally substituted by halo, deuterium, cycloalkyl, or OR^{a}; or
R₆ₐ and R_{6b} taken together with the atoms to which they are attached combine to form carbonyl (C═O), C₃₋₆ spirocyclic-cycloalkyl, or a 3-6 membered spirocyclic heterocyclyl comprising one or more O, N or S heteroatoms; wherein each hydrogen atom in cycloalkyl and heterocyclyl is optionally substituted by halo or OR^{a},
R^{a} is as defined above with respect to R₁ in Formula (I) or Formula (II-A), (II-B), (II-C) or (II-D); and
R₇ is H or C₁₋₄ alkyl.

In certain aspects, the invention relates to compounds of Formula (IV), or a pharmaceutically acceptable salt thereof:

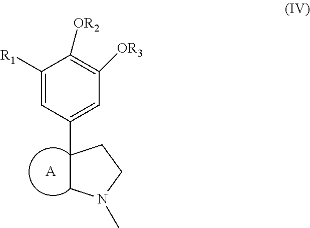

wherein
ring A is

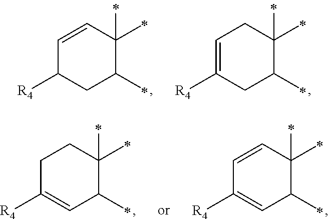

wherein * denotes the attachment points of ring A to the compound of formula (I), and wherein
each R₁ is independently halo, C₁₋₄ alkyl, C₁₋₄ haloalkyl, methoxy, acetyl, cyano, C₂₋₄ alkenyl, C₂₋₄ alkynyl, amino, amido or carboxyl;
each of R₂ and R₃ is independently H, or C₁₋₄ alkyl optionally substituted with one or more halo, C₃₋₆ cycloalkyl, alkenyl or alkynyl; or
R₂ and R₃ together with the atoms to which they are attached combine to form-(CR₆ₐR_{6b})ₓ—;
x is 1 or 2;
R₄ is methyl;
each of R₆ₐ and R_{6b} is independently H, C₁₋₄ alkyl, or C₁₋₄ haloalkyl; or
R₆ₐ and R_{6b} taken together with the atoms to which they are attached combine to form carbonyl (C═O), C₃₋₆ spirocyclic-cycloalkyl, or a 3-6 membered spirocyclic heterocyclyl comprising one or more O, N or S heteroatoms; wherein each hydrogen atom in cycloalkyl and heterocyclyl is optionally substituted by halo or OR^{a}; and
R^{a} is H, C₁₋₄ alkyl (e.g., methyl), C₁₋₄ haloalkyl or C₁₋₄ alkoxy.

### Ring A

In certain embodiments, the compound is a compound of Formula (I), Formula (II-A), Formula (II-B), Formula (II-C), or Formula (II-D), wherein ring A is

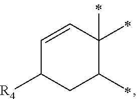

In certain embodiments, the compound is a compound of Formula (I), Formula (II-A), Formula (II-B), Formula (II-C), or Formula (II-D), wherein ring A is

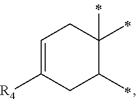

In certain embodiments, the compound is a compound of Formula (I), Formula (II-A), Formula (II-B), Formula (II-C), or Formula (II-D), wherein ring A is

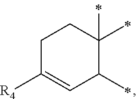

In certain embodiments, the compound is a compound of Formula (I), Formula (II-A), Formula (II-B), Formula (II-C), or Formula (II-D), wherein ring A is

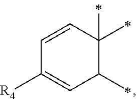

### R₁ and m

In certain embodiments, the compound is a compound of Formula (I), Formula (II-A), Formula (II-B), Formula (II-C), or Formula (II-D), or a pharmaceutically acceptable salt thereof, wherein each R₁ is independently hydrogen, halo, C₁₋₄ alkyl, or C₃₋₆ cycloalkyl; wherein each hydrogen atom in C₁₋₄ alkyl, or C₃₋₆ cycloalkyl is optionally substituted by halo or C₁₋₄ alkyl; and m is 0 or 1. In certain embodiments, the compound is a compound of Formula (I), Formula (II-A), Formula (II-B), Formula (II-C), or Formula (II-D), wherein: each R₁ is independently hydrogen, halo, methyl, ethyl, or cyclopropyl; wherein each hydrogen atom in methyl, ethyl, or cyclopropyl is optionally substituted by halo, methyl or halomethyl; and m is 0 or 1. In certain embodiments, the compound is a compound of Formula (I), Formula (II-A), Formula (II-B), Formula (II-C), or Formula (II-D), wherein each R₁ is independently hydrogen, fluoro, methyl optionally substituted with one or more fluoro, or cyclopropyl; and m is 0 or 1.

In certain embodiments, the compound is a compound of Formula (I), Formula (II-A), Formula (II-B), Formula (II-C), or Formula (II-D), wherein R₁ is halo. In certain embodiments, the compound is a compound of Formula (I), Formula (II-A), Formula (II-B), Formula (II-C), or Formula (II-D), wherein R₁ is fluoro. In certain embodiments, the compound is a compound of Formula (I), Formula (II-A), Formula (II-B), Formula (II-C), or Formula (II-D), wherein R₁ is cyclopropyl. In certain embodiments, the compound is a compound of Formula (I), Formula (II-A), Formula (II-B), Formula (II-C), or Formula (II-D), wherein m is 0 or 1.

In certain embodiments, R₁ is halo, haloalkyl, alkyl, alkenyl, alkynyl, cycloalkyl, cycloalkenyl, heterocycloalkyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}. In certain embodiments. R¹ is halo, haloalkyl, alkyl, cycloalkyl, cycloalkenyl, heterocycloalkyl, aryl, heteroaryl, —OR^{a} (e.g., alkoxy), —NR^{a}R^{b} (e.g., —NH₂), —CHO, —C(O)R^{a} (e.g., —C(O)alkyl, such as —C(O)CH₃), —CN, or nitro. In some embodiments, R₁ is halo, haloalkyl, alkyl, cycloalkyl, heterocycloalkyl, —OR^{a}, —NR^{a}R^{b}, —CHO, C(O)R^{a}, —CN, or nitro. In some further embodiments, R₁ is alkyl. In some further embodiments, R₁ is haloalkyl. In some embodiments, R₁ is acetyl. In some embodiments, R₁ is amido. In some embodiments, R₁ is —C(O) NH₂. In some embodiments, R₁ is cyano. In some embodiments, R₁ is C₁₋₄ alkenyl. In some embodiments, R₁ is C₁₋₄ alkynyl. In some embodiments, R₁ is C₁₋₄ alkoxy. In some embodiments, R₁ is C₁₋₄ methoxy. In some embodiments, R₁ is methyl. In some embodiments, R₁ is fluoro. In some embodiments, R₁ is (CH₂)—(C₃₋₆ cycloalkyl).

In certain embodiments, R₁ is halo, cycloalkyl, —OR^{a} (e.g., alkoxy), —NR^{a}R^{b} (e.g., —NH₂), C(O)R^{a} (e.g., —C(O)alkyl, such as —C(O)CH₃), —CN, or nitro. For example, R₁ can be halo, cycloalkyl, alkoxy, —NH₂, C(O)alkyl, —CN, or nitro. In some embodiments, R₁ is —C(O)alkyl, such as —C(O)CH₃. In some embodiments, R₁ is alkoxy, such as methoxy. In some embodiments, R₁ is cycloalkyl, such as cyclopropyl.

In certain embodiments, R₁ is halo, cyclopropyl, —OCH₃, —NH₂, —C(O)CH₃, —CN, or nitro. In some embodiments, R₁ is halo. In some embodiments, R₁ is —CN. In some embodiments, R₁ is nitro. In some embodiments, R₁ is —NH₂. In certain embodiments, R₁ is halo, haloalkyl, alkyl, cyclopropyl, —NH₂, —NO₂, —C(O)R^{a}, or —CN. In certain embodiments, R₁ is halo. In certain embodiments, R₁ is haloalkyl. In certain embodiments, R₁ is alkyl. In certain embodiments, R₁ is cyclopropyl. In certain embodiments, R₁ is —NH₂. In certain embodiments, R₁ is —NO₂. In certain embodiments. R₁ is —C(O)R^{a}, wherein R^{a} is hydrogen or alkyl. In certain embodiments, R₁ is —C(O)R^{a}, wherein R^{a} is hydrogen or methyl. In certain embodiments, R₁ is —CN.

In certain embodiments, each R^{a} and R^{b} is independently H, alkyl (e.g., methyl), alkenyl, cycloalkyl, heterocycloalkyl, aryl, or heteroaryl; or if an instance of R₁ is —NR^{a}R^{b}, then R^{a} and Rh may combine with the nitrogen atom to which they are attached to form heterocycloalkyl or heteroaryl. For example, if R₁ is —NR^{a}R^{b}, then R^{a} and R^{b} may combine with the nitrogen atom to which they are attached to form

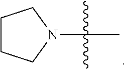 [SMILES: *N1CCCC1]

In certain embodiments, each R^{a} and R^{b} is independently H. In certain embodiments, each R^{a} and R^{b} is independently H or methyl.
R₂ and R₃

In certain embodiments, the compound is a compound of Formula (I), Formula (II-A), Formula (II-B), Formula (II-C), or Formula (II-D), wherein R₂ is halomethyl. In certain embodiments, the compound is a compound of Formula (I), Formula (II-A), Formula (II-B), Formula (II-C), or Formula (II-D), wherein R₂ is —CHF₂ or —CF₃.

In certain embodiments, the compound is a compound of Formula (I), Formula (II-A), Formula (II-B), Formula (II-C), or Formula (II-D), wherein R₂ and R₃ together with the atoms to which they are attached combine to form heterocyclyl or heteroaryl, wherein each hydrogen atom in heterocyclyl and heteroaryl is optionally substituted by halo or OR^{a}. In certain embodiments, each R^{a} is H, alkyl (e.g., methyl), alkenyl, cycloalkyl, heterocycloalkyl, aryl, or heteroaryl. In certain embodiments, R^{a} is H or C₁₋₄ alkyl or C₁₋₄ haloalkyl. In certain embodiments, R^{a} is H. In certain embodiments, each R^{a} is H or methyl optionally substituted with one or more fluoro. In certain embodiments, R^{a} is CF₃.

In certain embodiments, R₂ is methyl. In further embodiments, R₂ is halomethyl such as a methyl substituted with at least one fluoro (e.g., CHF₂ or CF₃).

In certain embodiments. R₃ is methyl. In further embodiments. R₃ is halomethyl such as a methyl substituted with at least one fluoro (e.g., CHF₂ or CF₃). In some embodiments, R₃ is benzyl.

In certain embodiments, the compound is a compound of Formula (I), Formula (II-A), Formula (II-B), Formula (II-C), or Formula (II-D), wherein each of R₂ and R₃ is C₁₋₄ alkyl optionally substituted with one or more halo. In certain embodiments, the compound is a compound of Formula (I), Formula (II-A), Formula (II-B), Formula (II-C), or Formula (II-D), wherein each of R₂ and R₃ is C₁₋₄ alkyl optionally substituted with one or more fluoro. In certain embodiments, the compound is a compound of Formula (I), Formula (II-A), Formula (II-B), Formula (II-C), or Formula (II-D), wherein each of R₂ and R₃ is C₁₋₄ alkyl optionally substituted with cyclopropyl, cyclobutyl or cyclohexyl. In certain embodiments, the compound is a compound of Formula (I), Formula (II-A), Formula (II-B), Formula (II-C), or Formula (II-D), wherein each of R₂ and R₃ is C₁₋₄ alkyl optionally substituted with cyclopropyl. In certain embodiments, the compound is a compound of Formula (I), Formula (II-A), Formula (II-B), Formula (II-C), or Formula (II-D), wherein each of R₂ and R₃ is C₁₋₄ alkyl optionally substituted with one or more C₁₋₄ alkoxy.

In certain embodiments, the compound is a compound of Formula (I), Formula (II-A), Formula (II-B), Formula (II-C), or Formula (II-D), wherein each of R₂ and R₃ is C₁₋₄ alkyl optionally substituted with phenyl. In certain embodiments, the compound is a compound of Formula (I), Formula (II-A), Formula (II-B), Formula (II-C), or Formula (II-D), wherein each of R₂ and R₃ is C₁₋₄ alkyl or benzyl. In certain embodiments, the compound is a compound of Formula (I), Formula (II-A), Formula (II-B), Formula (II-C), or Formula (II-D), wherein each of R₂ is methyl and R₃ is benzyl. In certain embodiments, the compound is a compound of Formula (I), Formula (II-A), Formula (II-B), Formula (II-C), or Formula (II-D), wherein each of R₂ is benzyl and R₃ is C₁₋₄ alkyl.

In certain embodiments, the compound is a compound of Formula (I), Formula (II-A), Formula (II-B), Formula (II-C), or Formula (II-D), wherein each of R₂ is C₁₋₄ alkyl optionally substituted with one or more halo and R₃ is C₁₋₄ alkyl optionally substituted with 5-6 membered heteroaryl optionally substituted with one or more C₁₋₄ alkyl, C₁₋₄ haloalkyl, C₁₋₄ alkoxy, cyano, amino, acetyl or amido.

In certain embodiments, the compound is a compound of Formula (I), Formula (II-A), Formula (II-B), Formula (II-C), or Formula (II-D), wherein each of R₂ is C₁₋₄ alkyl optionally substituted with one or more halo and R₃ is C₁₋₄ alkyl optionally substituted with one or more 5-6 membered heteroaryl optionally substituted with one or more C₁₋₄ alkyl, C₁₋₄ haloalkyl, C₁₋₄ alkoxy, cyano, amino, acetyl or amido.

In certain embodiments, the compound is a compound of Formula (I), Formula (II-A), Formula (II-B), Formula (II-C), or Formula (II-D), wherein each of R₂ and R₃ is C₁₋₄ alkyl optionally substituted with one or more fluoro.

In certain embodiments, the compound is a compound of Formula (I), Formula (II-A), Formula (II-B), Formula (II-C), or Formula (II-D), wherein each of R₂ is C₁₋₄ alkenyl and R₃ is C₁₋₄ alkyl. In certain embodiments, the compound is a compound of Formula (I), Formula (II-A), Formula (II-B), Formula (II-C), or Formula (II-D), wherein each of R₂ is C₁₋₄ alkynyl and R₃ is C₁₋₄ alkyl. In certain embodiments, the compound is a compound of Formula (I), Formula (II-A), Formula (II-B), Formula (II-C), or Formula (II-D), wherein each of R₃ is C₁₋₄ alkenyl and R₂ is C₁₋₄ alkyl. In certain embodiments, the compound is a compound of Formula (I), Formula (II-A), Formula (II-B), Formula (II-C), or Formula (II-D), wherein each of R₃ is C₁₋₄ alkynyl and R₂ is C₁₋₄ alkyl.

In certain embodiments, the compound is a compound of Formula (I), Formula (II-A), Formula (II-B), Formula (II-C), or Formula (II-D), wherein each of R₂ and R₃ is hydroxyl or C₁₋₄ alkyl optionally substituted with one or more halo. In certain embodiments, the compound is a compound of Formula (I), Formula (II-A), Formula (II-B), Formula (II-C), or Formula (II-D), wherein each of R₂ is hydroxyl and R₃ is C₁₋₄ alkyl optionally substituted with one or more halo. In certain embodiments, the compound is a compound of Formula (I), Formula (II-A), Formula (II-B), Formula (II-C), or Formula (II-D), wherein each of R₃ is hydroxyl and R₂ is C₁₋₄ alkyl optionally substituted with one or more halo.

### R₄

In certain embodiments, the compound is a compound of Formula (I), Formula (II-A), Formula (II-B), Formula (II-C), or Formula (II-D), wherein R₄ is H, C₁₋₄ alkyl, or C₁₋₆ haloalkyl. In certain embodiments, the compound is a compound of Formula (I), Formula (II-A), Formula (II-B), Formula (II-C), or Formula (II-D), wherein R₄ is methyl. In certain embodiments, the compound is a compound of Formula (I), Formula (II-A), Formula (II-B), Formula (II-C), or Formula (II-D), wherein C₁₋₄ alkyl.

### R₅

In certain embodiments, the compound is a compound of Formula (I), Formula (II-A), Formula (II-B), Formula (II-C), Formula (II-D), Formula (III) or Formula (IV) wherein R₅ is C₁₋₄ alkyl.

In certain embodiments, the compound is a compound of Formula (I), Formula (II-A), Formula (II-B), Formula (II-C), Formula (II-D), Formula (III) or Formula (IV), wherein R₅ is C₁-4 alkyl optionally substituted with C₃₋₆ cycloalkyl, 3-6 membered heterocycle, phenyl or 5-6 membered heteroaryl, wherein the alkyl, cycloalkyl, heterocycle, aryl, or heteroaryl are each independently optionally substituted with one or more deuterium, C₁₋₄ alkoxy, C₁₋₄ haloalkyl, halo, cyano, amino, carboxy, acetyl, or amide.

In certain embodiments, the compound is a compound of Formula (I), Formula (II-A), Formula (II-B), Formula (II-C), or Formula (II-D), wherein R₅ is C₁₋₄ alkyl optionally substituted with cyclopropyl, cyclobutyl, or cyclohexyl.

In certain embodiments, the compound is a compound of Formula (I), Formula (II-A), Formula (II-B), Formula (II-C), Formula (II-D), Formula (III) or Formula (IV) wherein R₅ is C₁₋₄ alkyl optionally substituted with phenyl optionally substituted with one or more methyl optionally substituted with one or more halo, or methoxy.

In certain embodiments, the compound is a compound of Formula (I), Formula (II-A), Formula (II-B), Formula (II-C), Formula (II-D), Formula (III) or Formula (IV), wherein R₅ is C₁-4 alkyl optionally substituted with one or more fluoro. In certain embodiments, the compound is a compound of Formula (I), Formula (II-A), Formula (II-B), Formula (II-C), Formula (II-D), Formula (III) or Formula (IV), wherein R₅ is C₁₋₄ alkyl optionally substituted with one or more deuterium.

R₆ₐ and R_{6b} and x

In certain embodiments, the compound is a compound of Formula (III), wherein x is 1. In certain embodiments, the compound is a compound of Formula (III), wherein x is 2.

In certain embodiments, the compound is a compound of Formula (III) wherein each of R₆ₐ and R_{6b} is independently H, halo or optionally substituted C₁₋₄ alkyl, wherein each hydrogen atom in the C₁₋₄ alkyl is optionally substituted by halo, deuterium, C₃₋₆ cycloalkyl, or OR^{a}, wherein R^{a} is as defined herein with respect to Formula (I). In certain embodiments, the compound is a compound of Formula (III) wherein each of R₆ₐ and R_{6b} is independently H or optionally substituted C₁₋₄ alkyl, wherein each hydrogen atom in the C₁₋₄ alkyl is optionally substituted by halo, deuterium, C₃₋₆ cycloalkyl, or OR^{a}, wherein R^{a} is C₁₋₄ alkyl optionally substituted by halo or C₁₋₄ alkoxy. In certain embodiments, the compound is a compound of Formula (III), wherein each of R₆ₐ and R_{6b} is independently H or optionally substituted C₁₋₄ alkyl, wherein each hydrogen atom in the C₁₋₄ alkyl is optionally substituted by halo, C₃₋₆ cycloalkyl, C₁₋₄ alkoxy, or C₁₋₄ haloalkyl.

In certain embodiments, the compound is a compound of Formula (III), wherein R₆ₐ and R_{6b} taken together with the atoms to which they are attached combine to form carbonyl (C═O), C₃₋₆ spirocyclic-cycloalkyl, or a 3-6 membered spirocyclic heterocyclyl comprising one or more O, N or S heteroatoms; wherein each hydrogen atom in cycloalkyl and heterocyclyl is optionally substituted by halo or OR^{a}, wherein R^{a} is C₁₋₄ alkyl. In certain embodiments, the compound is a compound of Formula (III), wherein R₆ₐ and R_{6b} taken together with the atoms to which they are attached combine to form carbonyl (C═O), C₃₋₆ spirocyclic-cycloalkyl, or a 3-6 membered spirocyclic heterocyclyl comprising one or more O, N or S heteroatoms; wherein each hydrogen atom in cycloalkyl and heterocyclyl is optionally substituted by halo or OR^{a}, wherein R^{a} is methyl. In certain embodiments, the compound is a compound of Formula (III), wherein each of R₆ₐ and R_{6b} is independently optionally substituted C₁₋₄ alkyl, wherein each hydrogen atom in the alkyl is optionally substituted by fluoro, C₃₋₆ cycloalkyl, or OR^{a}; wherein R^{a} is C₁₋₄ alkyl, C₁₋₄ haloalkyl, or C₁₋₄ alkoxy.

In certain embodiments, the compound is a compound of Formula (III) wherein each of R₆ₐ and R_{6b} is independently hydrogen, halo, C₁₋₄ alkyl, or C₁₋₄ haloalkyl. In certain embodiments, the compound is a compound of Formula (III), wherein R₆ₐ and R_{6b} are each H. In certain embodiments, the compound is a compound of Formula (III), wherein R₆ₐ and R_{6b} are each fluoro. In certain embodiments, the compound is a compound of Formula (III), wherein R₆ₐ and R_{6b} are each methyl. In certain embodiments, the compound is a compound of Formula (III), wherein each of R₆ₐ and R_{6b} is independently hydrogen, fluoro, or C₁₋₄ alkyl optionally substituted with one or more fluoro. In certain embodiments, the compound is a compound of Formula (III), wherein each of R₆ₐ and R_{6b} is independently hydrogen, fluoro, or methyl optionally substituted with one or more fluoro.

In certain embodiments, the compound is a compound of Formula (III), wherein R₆ₐ and R_{6b} taken together with the atoms to which they are attached combine to form carbonyl (C═O).

In certain embodiments, the compound is a compound of Formula (III), wherein R₆ₐ and R_{6b} taken together with the atoms to which they are attached combine to form C₃₋₆ spirocyclic-cycloalkyl. In certain embodiments, the compound is a compound of Formula (III) wherein R₆ₐ and R_{6b} taken together with the atoms to which they are attached combine to form C₃₋₆ spirocyclic-cyclopropyl. In certain embodiments, the compound is a compound of Formula (III) wherein R₆ₐ and R_{6b} taken together with the atoms to which they are attached combine to form C₃₋₆ spirocyclic-cyclobutyl. In certain embodiments, the compound is a compound of Formula (III), wherein R₆ₐ and R_{6b} taken together with the atoms to which they are attached combine to form C₃₋₆ spirocyclic-cyclohexyl.

In certain embodiments, the compound is a compound of Formula (III) wherein R₆ₐ and R_{6b} taken together with the atoms to which they are attached combine to form a 3-6 membered spirocyclic heterocyclyl comprising one or more O, N or S heteroatoms; wherein each hydrogen atom in cycloalkyl and heterocyclyl is optionally substituted by halo or OR^{a}, wherein R^{a} is C₁₋₄ alkyl, C₁₋₄ haloalkyl, or C₁₋₄ alkoxy. In certain embodiments, the compound is a compound of Formula (III), wherein R₆ₐ and R_{6b} taken together with the atoms to which they are attached combine to form a lactone or a lactam.

In certain embodiments, the compound is a compound of Formula (III), wherein R₆ₐ and R_{6b} taken together with the atoms to which they are attached combine to form an alpha-lactone. In certain embodiments, the compound is a compound of Formula (III), wherein R₆ₐ and R_{6b} taken together with the atoms to which they are attached combine to form a beta-lactone. In certain embodiments, the compound is a compound of Formula (III), wherein R₆ₐ and R_{6b} taken together with the atoms to which they are attached combine to form a gamma-lactone. In certain embodiments, the compound is a compound of Formula (III), wherein R₆ₐ and R_{6b} taken together with the atoms to which they are attached combine to form a delta-lactone. In certain embodiments, the compound is a compound of Formula (III), wherein R₆ₐ and R_{6b} taken together with the atoms to which they are attached combine to form a gamma-lactone.

In certain embodiments, the compound is a compound of Formula (III), wherein R₆ₐ and R_{6b} taken together with the atoms to which they are attached combine to form a alpha-lactam. In certain embodiments, the compound is a compound of Formula (III), wherein R₆ₐ and R_{6b} taken together with the atoms to which they are attached combine to form a beta-lactam. In certain embodiments, the compound is a compound of Formula (III), wherein R₆ₐ and R_{6b} taken together with the atoms to which they are attached combine to form a gamma-lactam. In certain embodiments, the compound is a compound of Formula (III), wherein R₆ₐ and R_{6b} taken together with the atoms to which they are attached combine to form a capro-lactam.

### R₇

In certain embodiments, the compound is a compound of Formula (I), Formula (II-A), Formula (II-B), Formula (II-C), Formula (II-D), Formula (III) or Formula (IV), wherein R₇ is H. In certain embodiments, the compound is a compound of Formula (I), Formula (II-A), Formula (II-B), Formula (II-C), Formula (II-D), Formula (III) or Formula (IV), wherein R₇ is C₁₋₄ alkyl. In certain embodiments, the compound is a compound of Formula (I), Formula (II-A), Formula (II-B), Formula (II-C), Formula (II-D), Formula (III) or Formula (IV), wherein R₇ is methyl.

### Additional Compounds

In certain embodiments, the compound is a compound of Formula (I), Formula (II-A), Formula (II-B), Formula (II-C), or Formula (II-D), wherein R₂ and R₃ together with the atoms to which they are attached combine to form heterocyclyl or heteroaryl, wherein each hydrogen atom in heterocyclyl and heteroaryl is optionally substituted by halo or OR^{a}; R₄ is H, C₁₋₄ alkyl or C₁₋₆ haloalkyl; and R₅ is C₁₋₄ alkyl. In certain embodiments, the compound is a compound of Formula (I), Formula (II-A), Formula (II-B), Formula (II-C), or Formula (II-D), wherein each R₁ is independently hydrogen, halo, C₁₋₄ alkyl, or C₃₋₆ cycloalkyl; wherein each hydrogen atom in C₁₋₄ alkyl, or C₃₋₆ cycloalkyl is optionally substituted by halo or C₁₋₄ alkyl; m is 0 or 1; R₂ and R₃ together with the atoms to which they are attached combine to form a 5-6 membered heterocyclyl, wherein each hydrogen atom in heterocyclyl is optionally substituted by halo, C₁₋₄ alkyl or C₁₋₄ haloalkyl; R₄ is H. C₁₋₄ alkyl or C₁₋₄ haloalkyl; and R₅ is C₁₋₂ alkyl. In certain embodiments, the compound is a compound of Formula (I), Formula (II-A), Formula (II-B), Formula (II-C), or Formula (II-D), wherein: each R₁ is independently hydrogen, halo, methyl, ethyl, or cyclopropyl; wherein each hydrogen atom in methyl, ethyl, or cyclopropyl is optionally substituted by halo, methyl or halomethyl; m is 0 or 1; R₂ and R₃ together with the atoms to which they are attached combine to form a 5-6 membered heterocyclyl, wherein each hydrogen atom in heterocyclyl is optionally substituted by halo, C₁₋₄ alkyl or C₁₋₄ haloalkyl; R₄ is H, C₁₋₄ alkyl or C₁₋₄ haloalkyl; and R₅ is methyl. In certain embodiments, the compound is a compound of Formula (I), Formula (II-A), Formula (II-B), Formula (II-C), or Formula (II-D), wherein each R₁ is independently hydrogen, fluoro, methyl optionally substituted with one or more fluoro, or cyclopropyl; m is 0 or 1; R₂ and R₃ together with the atoms to which they are attached combine to form a 5-6 membered heterocyclyl, wherein each hydrogen atom in heterocyclyl is optionally substituted by fluoro or methyl optionally substituted with one or more fluoro; R₄ is H. C₁₋₄ alkyl or C₁₋₄ haloalkyl; and R₅ is methyl.

In certain embodiments, the compound is a compound of Formula (I), Formula (II-A), Formula (II-B), Formula (II-C), or Formula (II-D), wherein each R₁ is halo (e.g., fluoro) or cyclopropyl; m is 0 or 1; R₂ and R₃ together with the atoms to which they are attached combine to form a 5-membered heterocyclyl, wherein each hydrogen atom in heterocyclyl is optionally substituted by fluoro or methyl optionally substituted with one or more fluoro; R₄ is H, C₁₋₄ alkyl or C₁₋₄ haloalkyl; and R₅ is methyl. In certain embodiments, the compound is a compound of Formula (I), Formula (II-A), Formula (II-B), Formula (II-C), or Formula (II-D), wherein each R₁ is halo (e.g., fluoro) or cyclopropyl; m is 0 or 1; R₂ and R₃ together with the atoms to which they are attached combine to form a 5-membered heterocyclyl, wherein each hydrogen atom in heterocyclyl is optionally substituted by fluoro or methyl optionally substituted with one or more fluoro; R₄ is methyl; and R₅ is methyl.

In certain embodiments, the compound is a compound of Formula (I), Formula (II-A), Formula (II-B), Formula (II-C), or Formula (II-D), wherein each R₁ is halo (e.g., fluoro) or cyclopropyl; m is 0 or 1; R₂ and R₃ together with the atoms to which they are attached combine to form a 6-membered heterocyclyl, wherein each hydrogen atom in heterocyclyl is optionally substituted by fluoro or methyl optionally substituted with one or more fluoro; R₄ is H, C₁₋₄ alkyl or C₁₋₄ haloalkyl; and R₅ is methyl. In certain embodiments, the compound is a compound of Formula (I), Formula (II-A), Formula (II-B), Formula (II-C), or Formula (II-D), wherein each R₁ is halo (e.g., fluoro) or cyclopropyl; m is 0 or 1; R₂ and R₃ together with the atoms to which they are attached combine to form a 6-membered heterocyclyl, wherein each hydrogen atom in heterocyclyl is optionally substituted by fluoro or methyl optionally substituted with one or more fluoro; R₄ is methyl; and R₅ is methyl.

In certain embodiments, the compound is a compound of Formula (I), Formula (II-A), Formula (II-B), Formula (II-C), or Formula (II-D), wherein R₃ is methyl.

In certain embodiments, the compound is a compound of Formula (I), Formula (II-A), Formula (II-B), Formula (II-C), or Formula (II-D), wherein R₂ is methyl or halomethyl; and R₃ is methyl, halomethyl, or benzyl.

In certain embodiments, the compound is a compound of Formula (I), Formula (II-A), Formula (II-B), Formula (II-C), or Formula (II-D), wherein R₄ is methyl.

In certain embodiments, the compound is a compound of Formula (I), Formula (II-A), Formula (II-B), Formula (II-C), or Formula (II-D), wherein R₅ is methyl.

In certain embodiments, the compound is a compound of Formula (III) wherein

In certain embodiments, the compound is

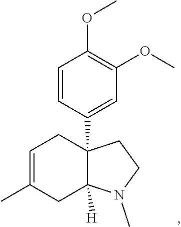

or a pharmaceutically acceptable salt thereof.

In certain embodiments, the compound is

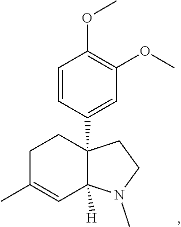

or a pharmaceutically acceptable salt thereof.

In certain embodiments, the compound is

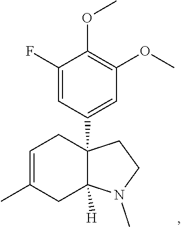

or a pharmaceutically acceptable salt thereof.

In certain embodiments, the compound is

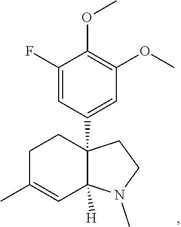

or a pharmaceutically acceptable salt thereof.

In certain embodiments, the compound is

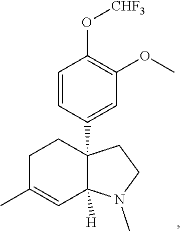

or a pharmaceutically acceptable salt thereof.

In certain embodiments, the compound is

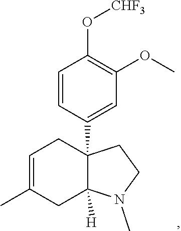

or a pharmaceutically acceptable salt thereof.

In certain embodiments, the compound is

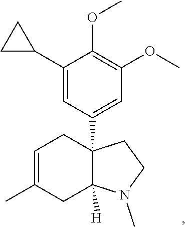 [SMILES: COc1cc([C@@]23CC=C(C)C[C@@H]2N(C)CC3)cc(C2CC2)c1OC]

or a pharmaceutically acceptable salt thereof.

In certain embodiments, the compound is

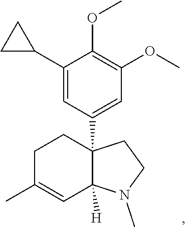 [SMILES: COc1cc([C@@]23CCC(C)=C[C@@H]2N(C)CC3)cc(C2CC2)c1OC]

or a pharmaceutically acceptable salt thereof.

In certain embodiments, the compound is

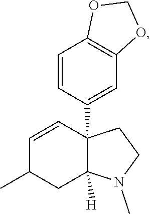 [SMILES: CC1C=C[C@@]2(c3ccc4c(c3)OCO4)CCN(C)[C@H]2C1]

or a pharmaceutically acceptable salt thereof.

In certain embodiments, the compound is

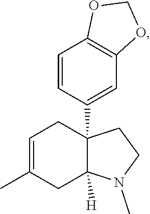 [SMILES: CC1=CC[C@@]2(c3ccc4c(c3)OCO4)CCN(C)[C@H]2C1]

or a pharmaceutically acceptable salt thereof.

In certain embodiments, the compound is

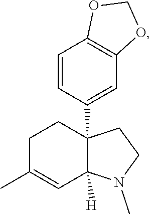 [SMILES: CC1=C[C@@H]2N(C)CC[C@]2(c2ccc3c(c2)OCO3)CC1]

or a pharmaceutically acceptable salt thereof.

In certain embodiments, the compound is

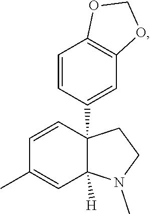 [SMILES: CC1=C[C@@H]2N(C)CC[C@]2(c2ccc3c(c2)OCO3)C=C1]

or a pharmaceutically acceptable salt thereof.

In some embodiments, the compound is selected from:

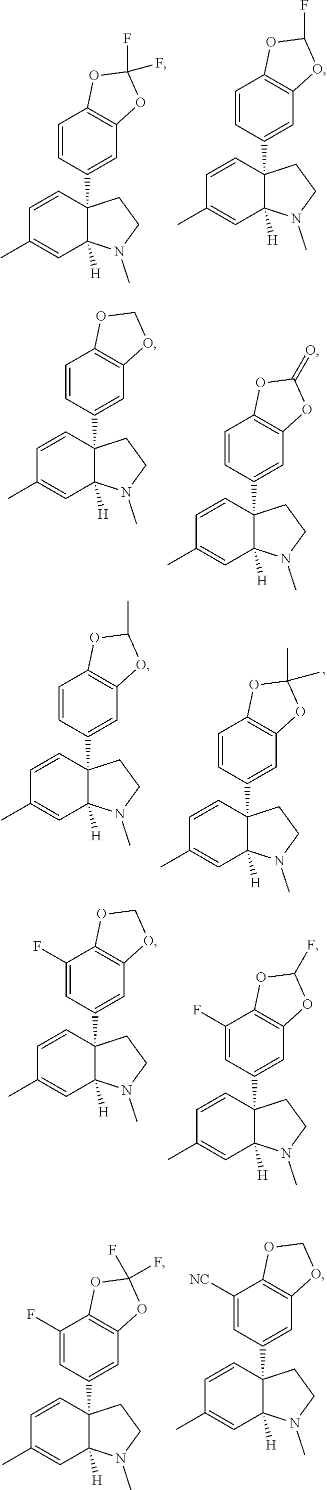

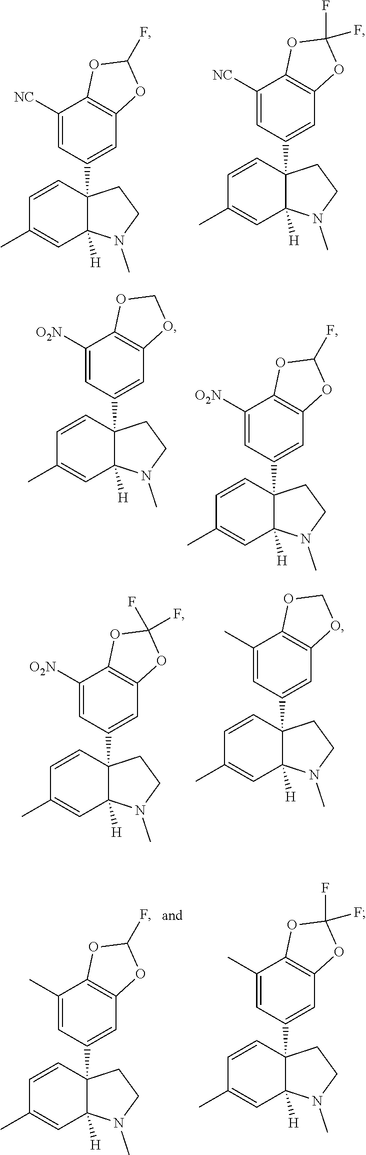

or a pharmaceutically acceptable salt thereof.

In some embodiments, the compound is selected from:

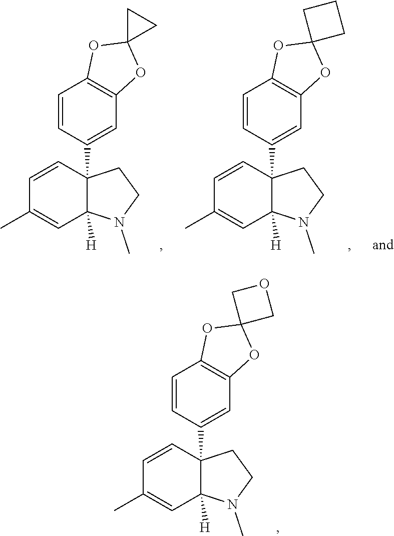

or a pharmaceutically acceptable salt thereof.

In some embodiments, the compound is selected from:

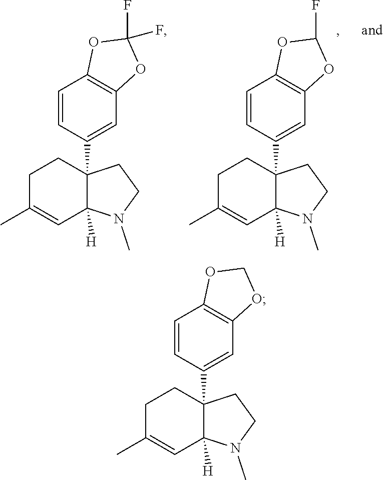

or a pharmaceutically acceptable salt thereof.

In some embodiments, the compound is selected from:

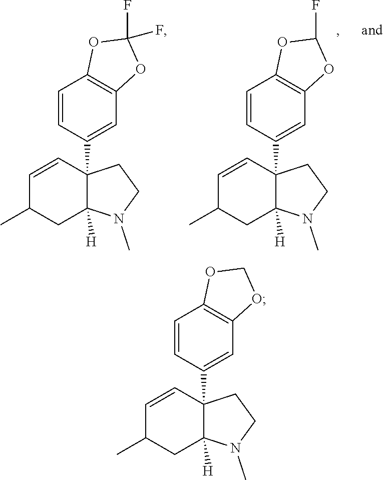

In some embodiments, the compound is selected from:

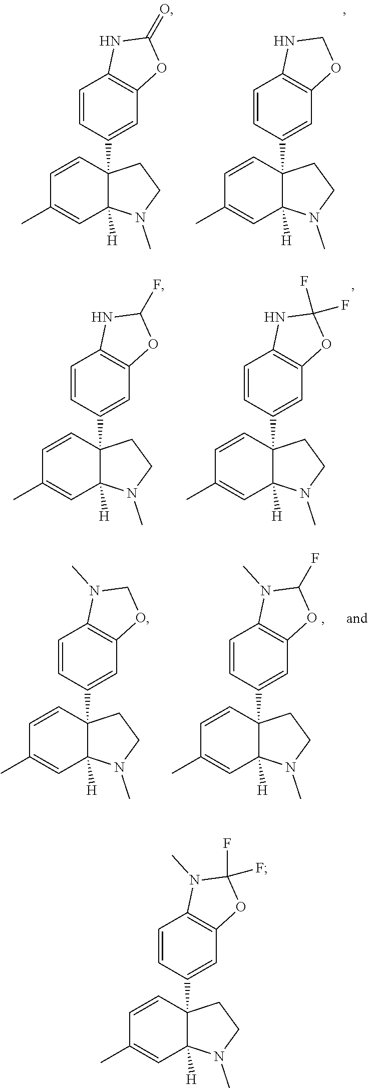

or a pharmaceutically acceptable salt thereof.

In some embodiments, the compound is selected from:

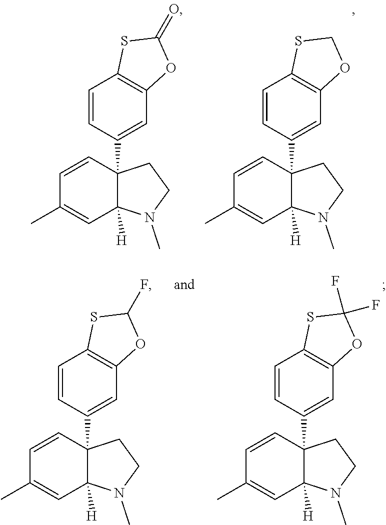

or a pharmaceutically acceptable salt thereof.

In some embodiments, the compound is selected from:

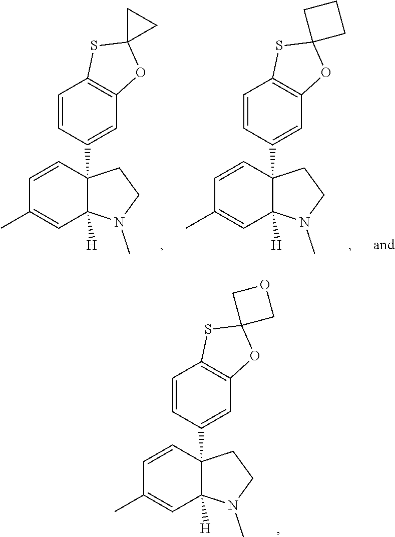

or a pharmaceutically acceptable salt thereof.

In some embodiments, the compound is selected from:

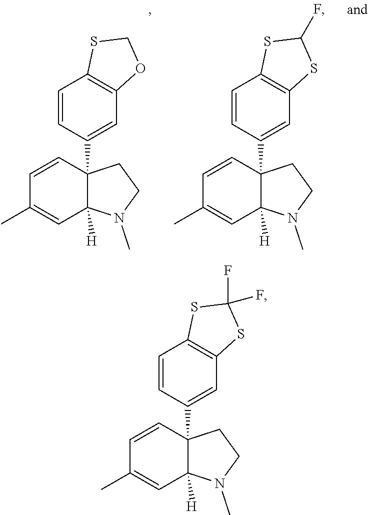

or a pharmaceutically acceptable salt thereof.

In some embodiments, the compound is selected from:

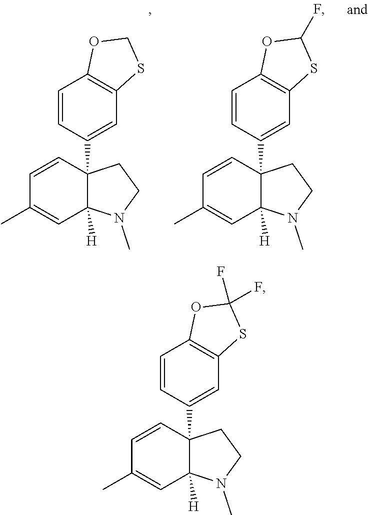

or a pharmaceutically acceptable salt thereof.

In some embodiments, the compound is selected from:

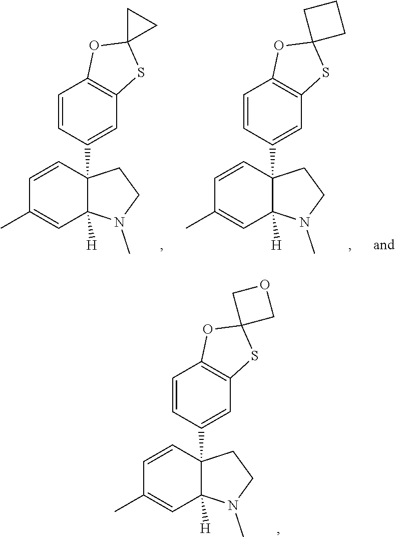

or a pharmaceutically acceptable salt thereof.

In some embodiments, the compound is selected from:

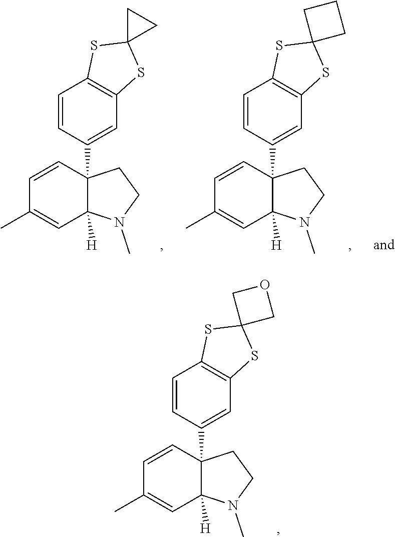

or a pharmaceutically acceptable salt thereof.

In some embodiments, the compound is selected from:

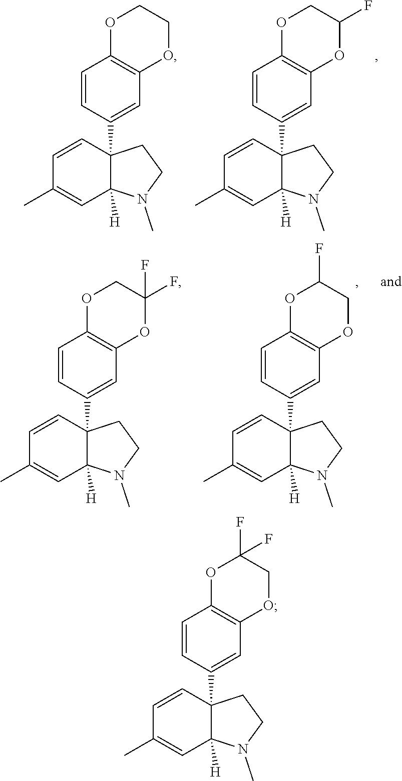

or a pharmaceutically acceptable salt thereof.

In some embodiments, the compound is a compound selected from:

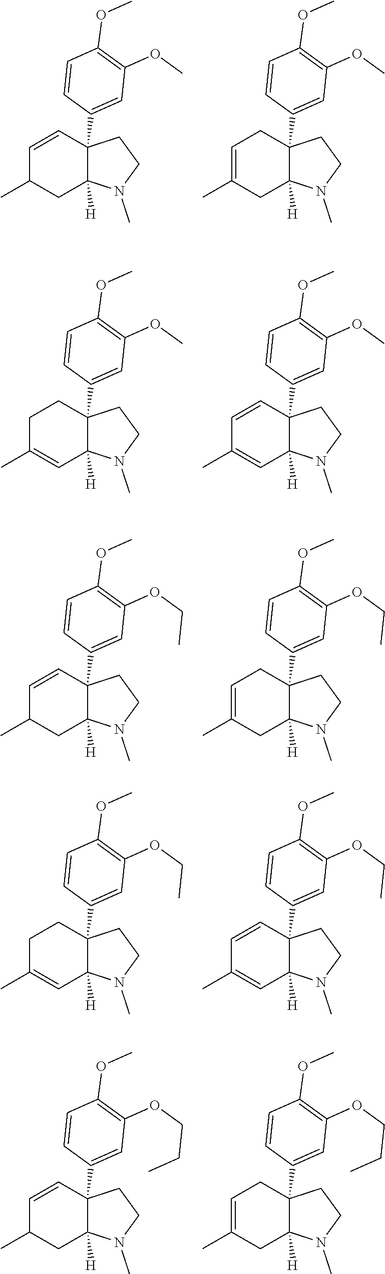

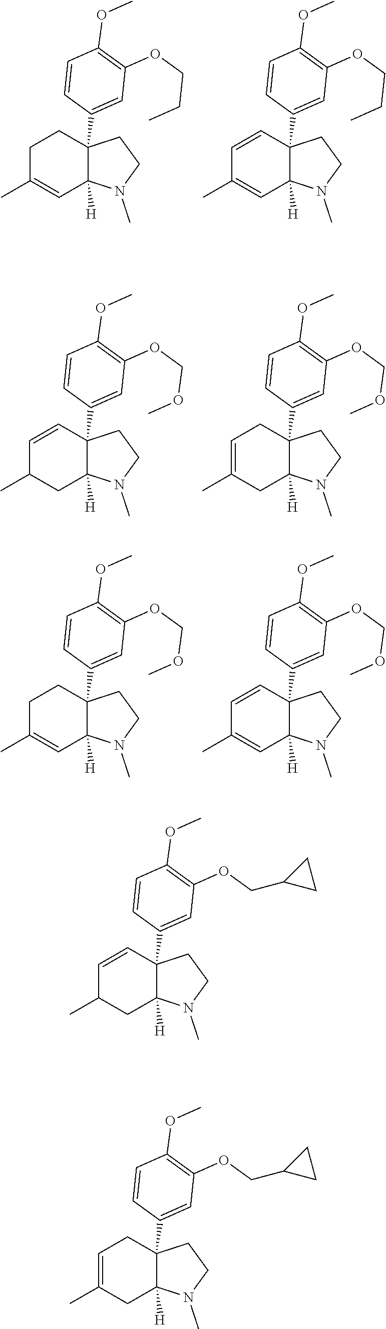

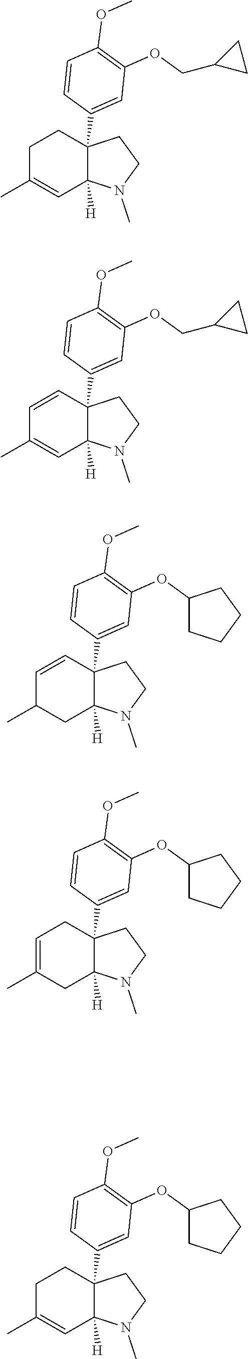

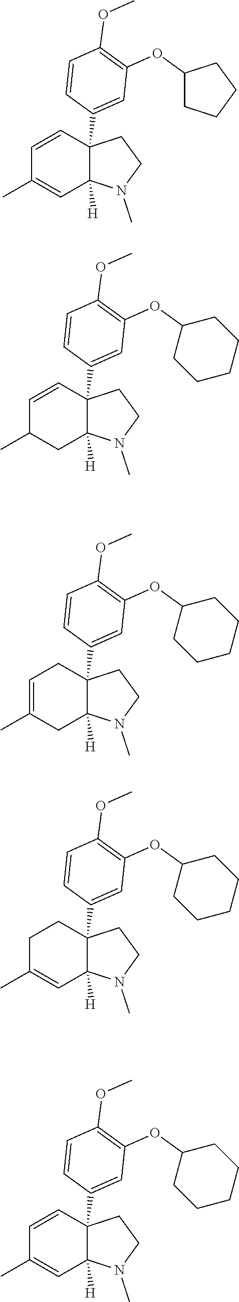

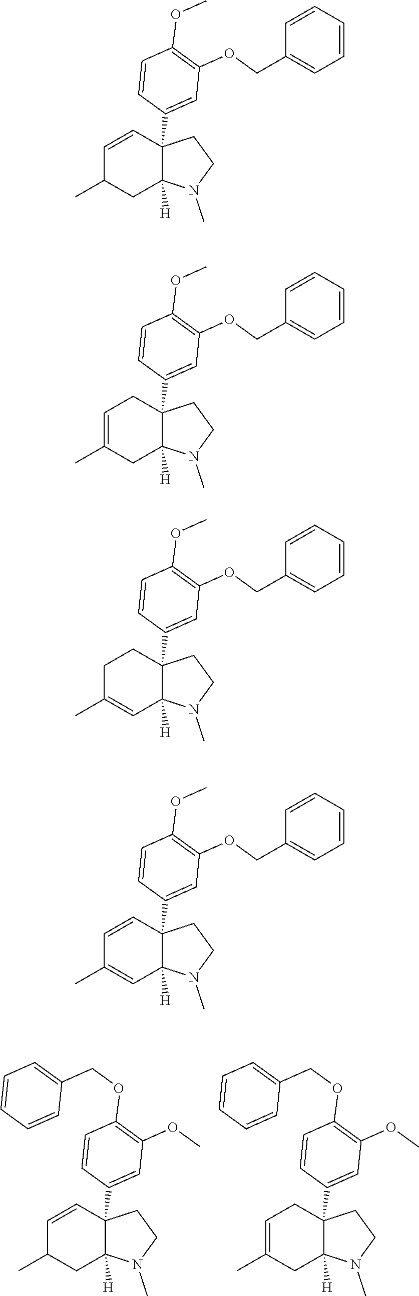

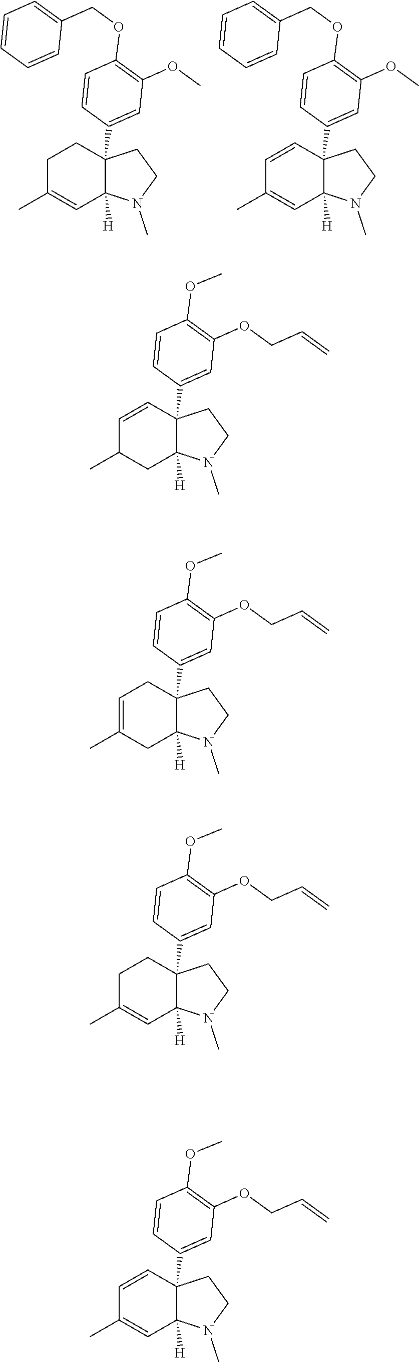

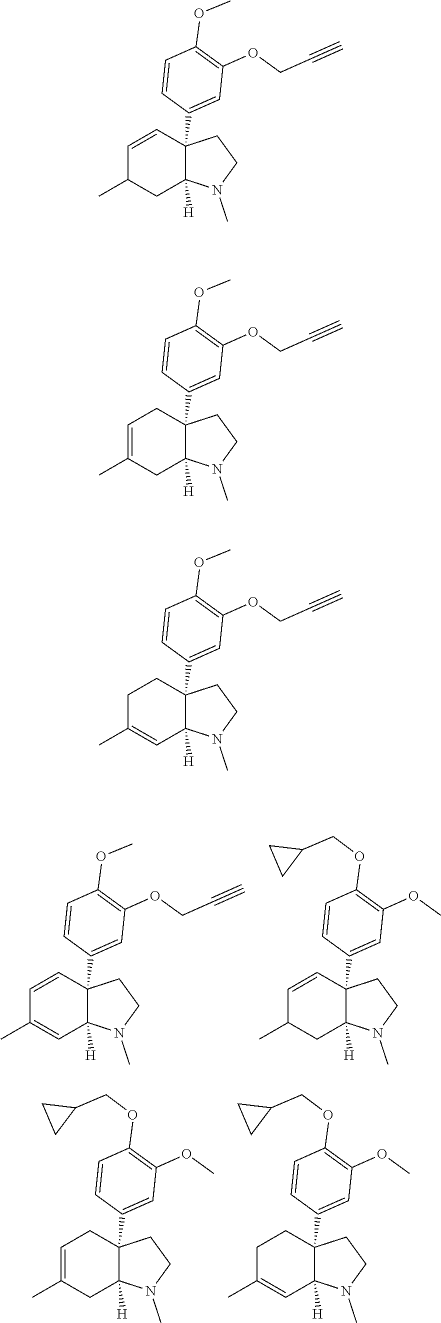

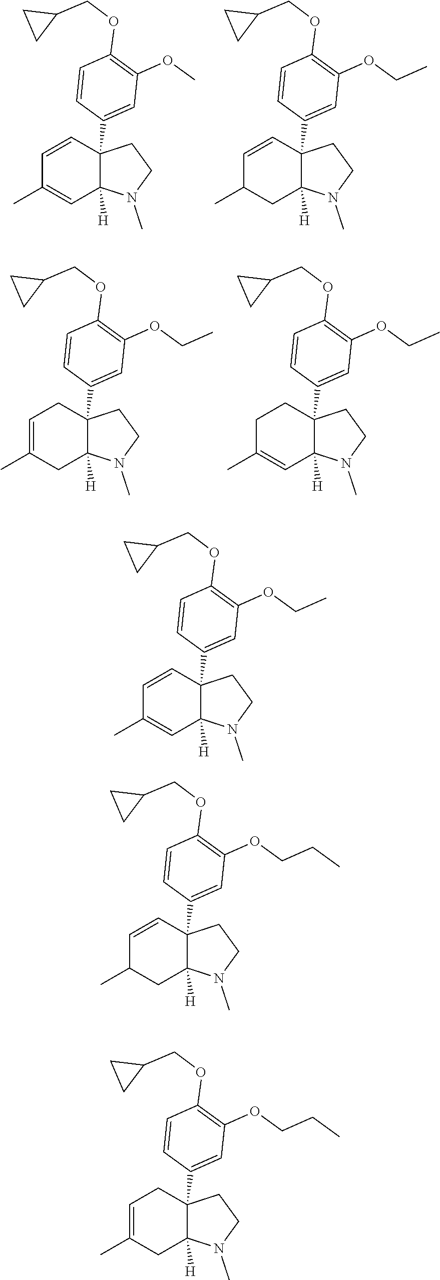

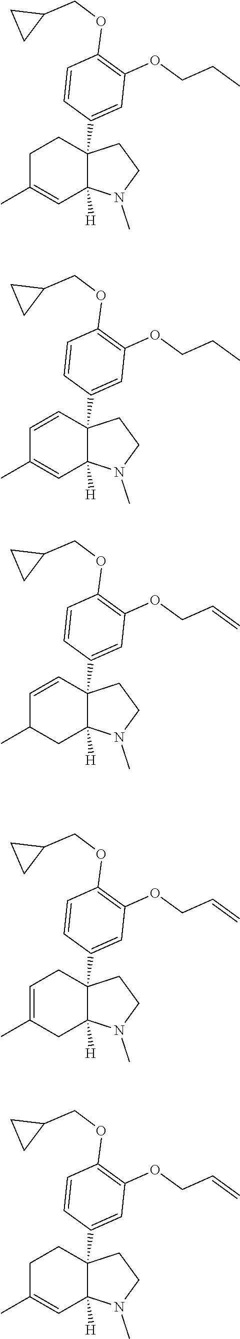

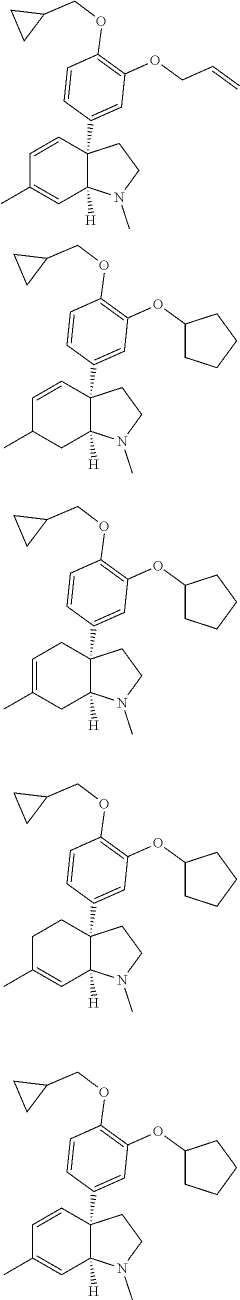

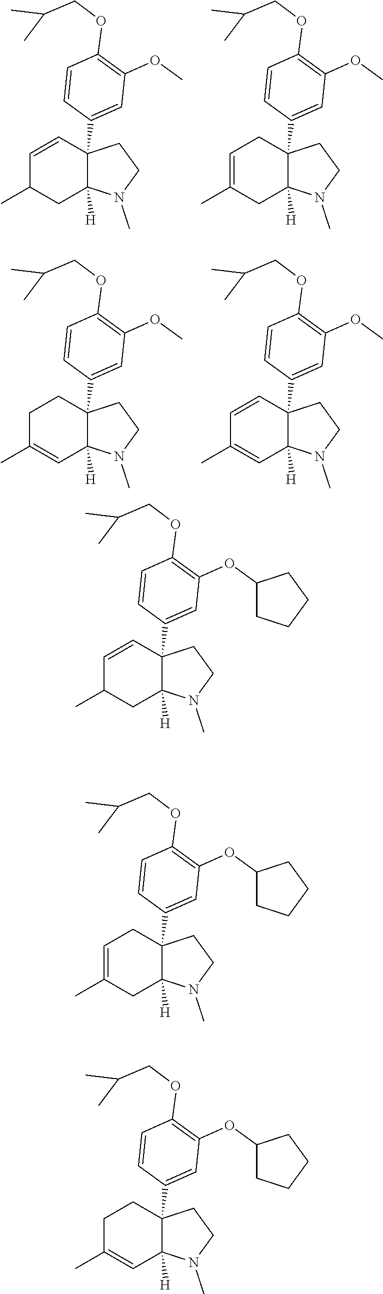

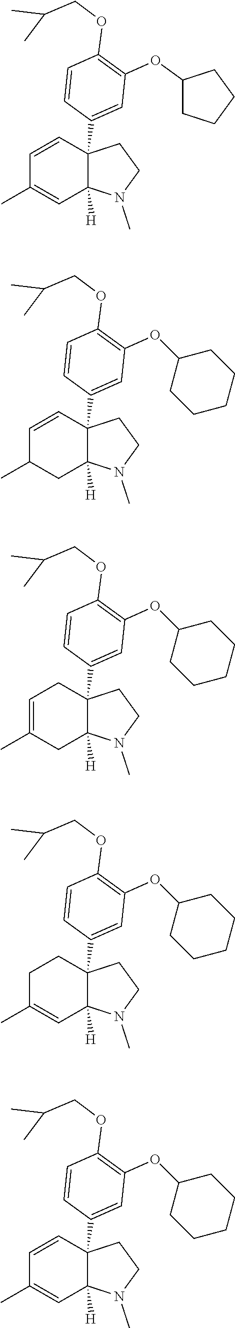 [SMILES: CC1=C[C@@H]2N(C)CC[C@]2(c2ccc(OCC(C)C)c(OC3CCCC3)c2)C=C1 ; CC(C)COc1ccc([C@@]23C=CC(C)C[C@@H]2N(C)CC3)cc1OC1CCCCC1 ; CC1=CC[C@@]2(c3ccc(OCC(C)C)c(OC4CCCCC4)c3)CCN(C)[C@H]2C1 ; CC1=C[C@@H]2N(C)CC[C@]2(c2ccc(OCC(C)C)c(OC3CCCCC3)c2)CC1 ; CC1=C[C@@H]2N(C)CC[C@]2(c2ccc(OCC(C)C)c(OC3CCCCC3)c2)C=C1]

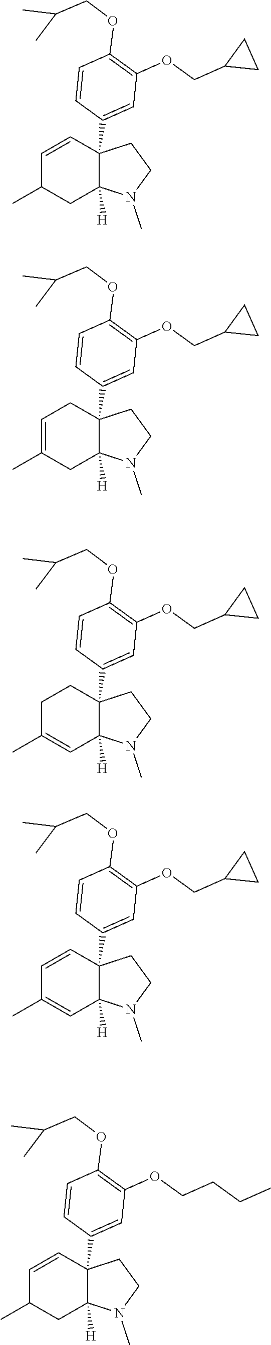 [SMILES: CC(C)COc1ccc([C@@]23C=CC(C)C[C@@H]2N(C)CC3)cc1OCC1CC1 ; CC1=CC[C@@]2(c3ccc(OCC(C)C)c(OCC4CC4)c3)CCN(C)[C@H]2C1 ; CC1=C[C@@H]2N(C)CC[C@]2(c2ccc(OCC(C)C)c(OCC3CC3)c2)CC1 ; CC1=C[C@@H]2N(C)CC[C@]2(c2ccc(OCC(C)C)c(OCC3CC3)c2)C=C1 ; CCCCOc1cc([C@@]23C=CC(C)C[C@@H]2N(C)CC3)ccc1OCC(C)C]

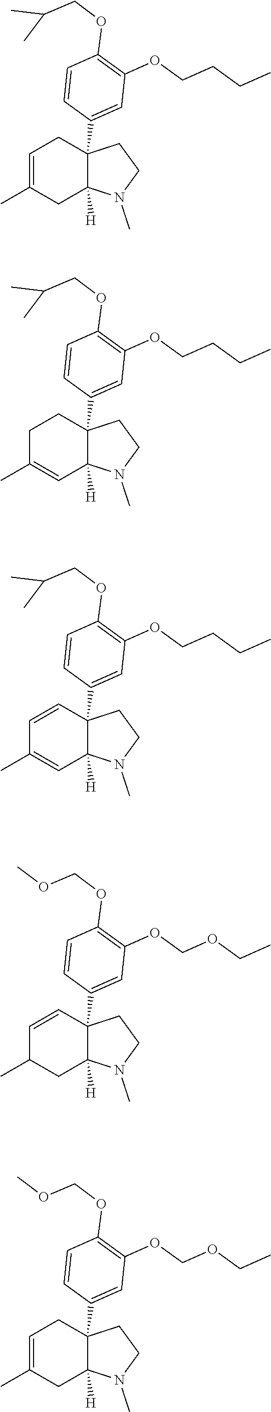

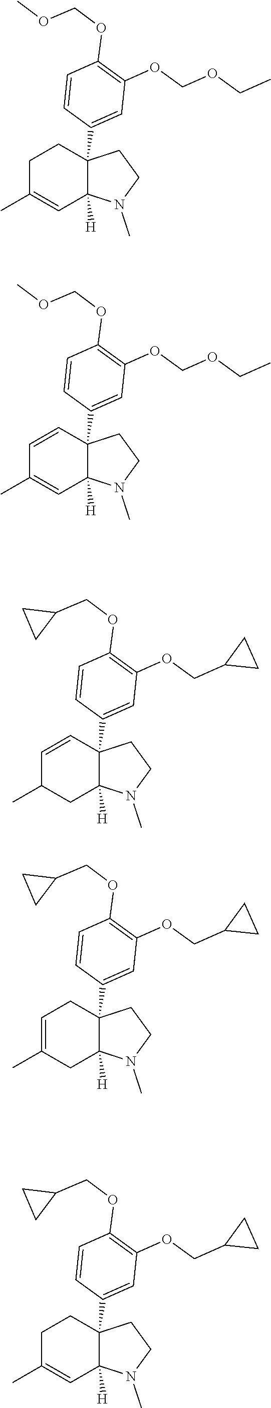

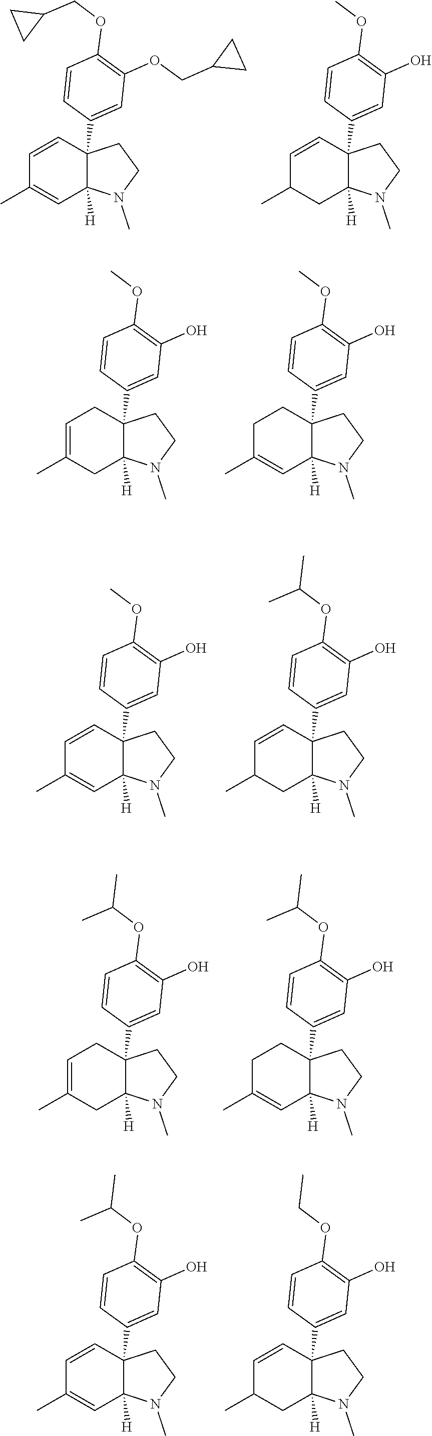

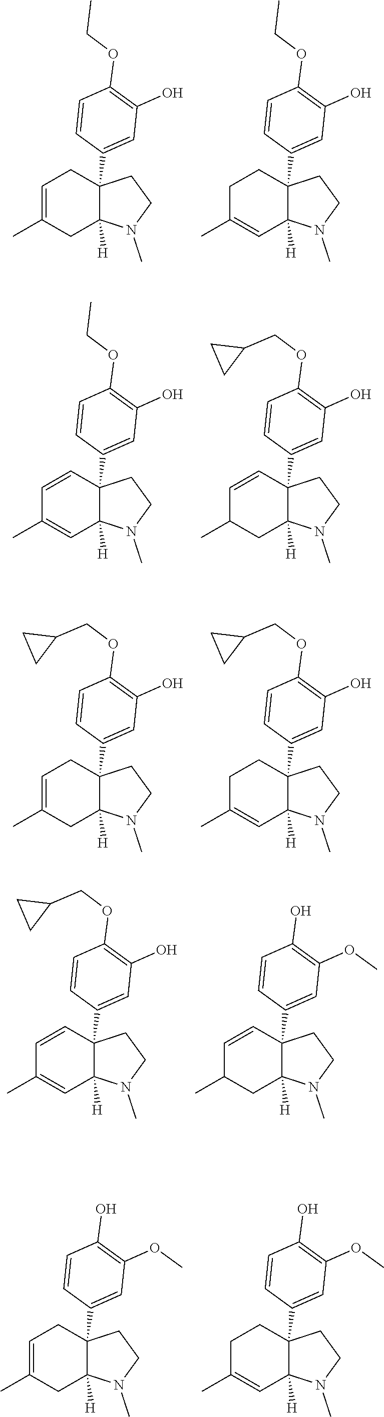

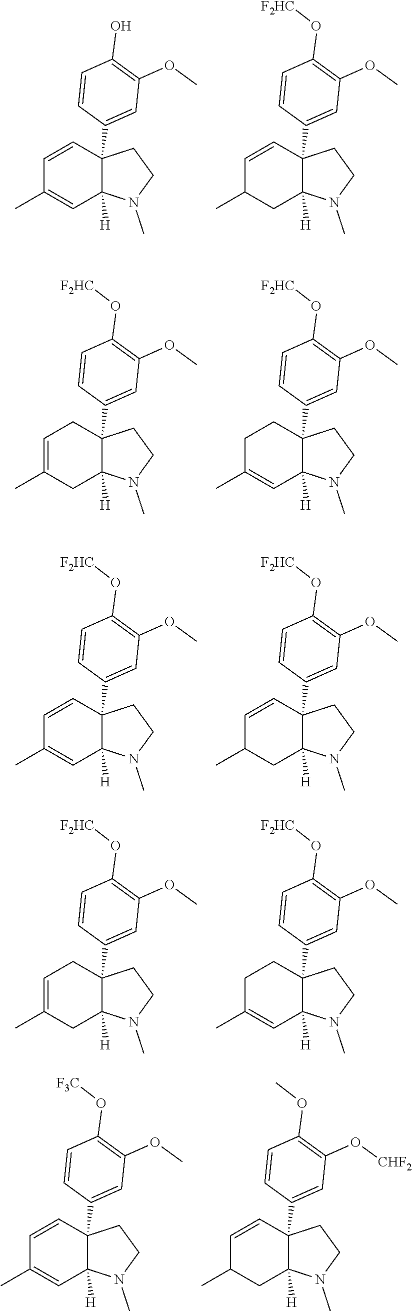

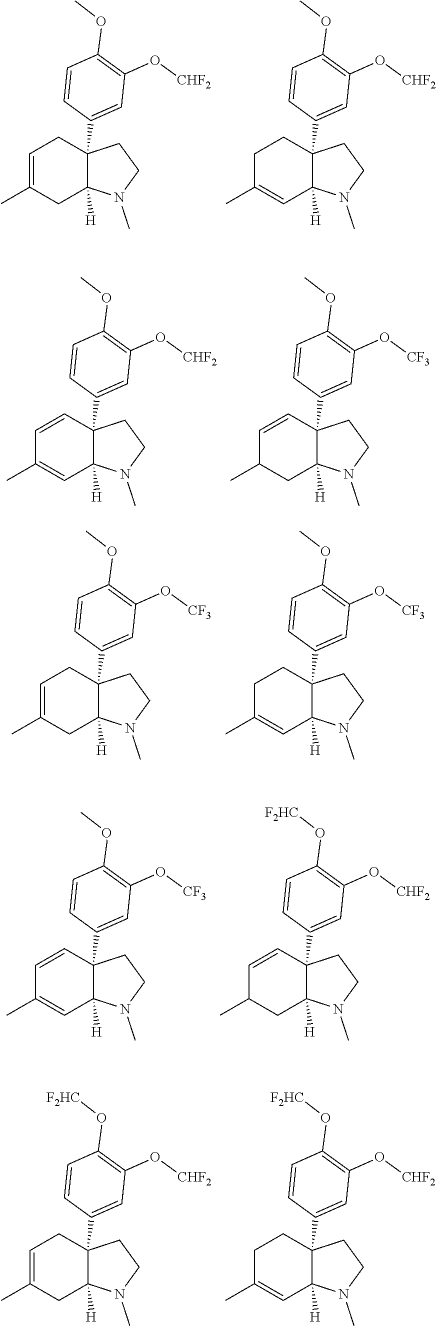

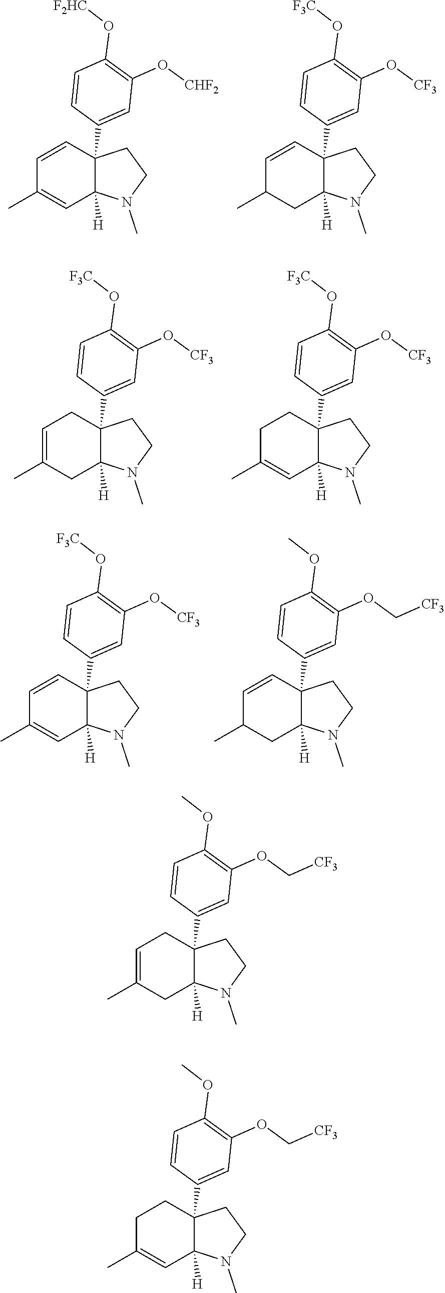

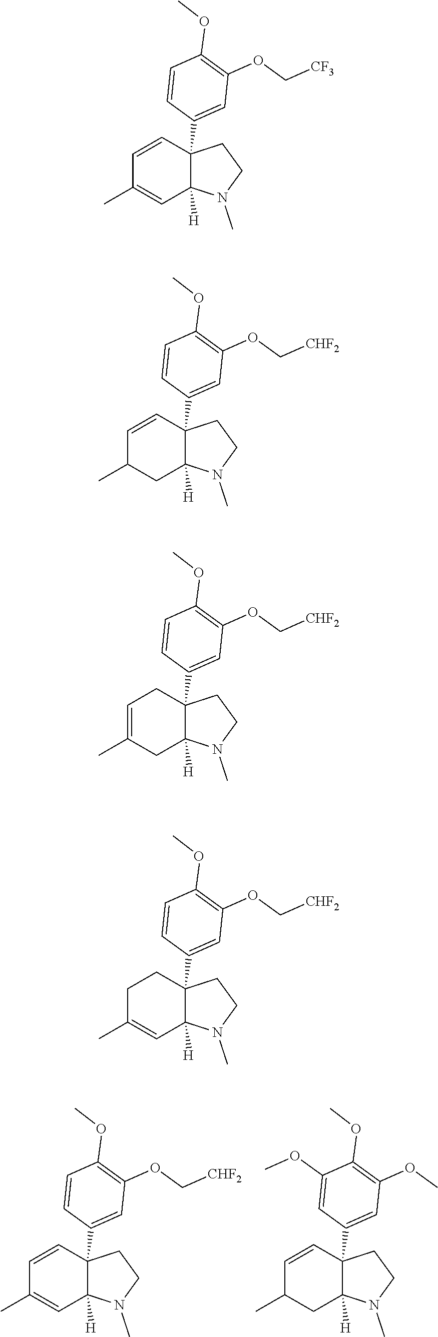

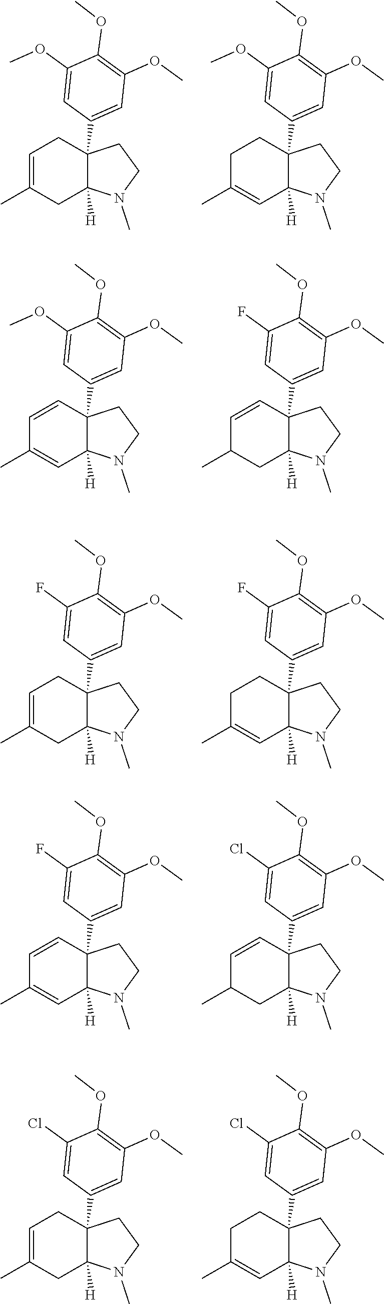

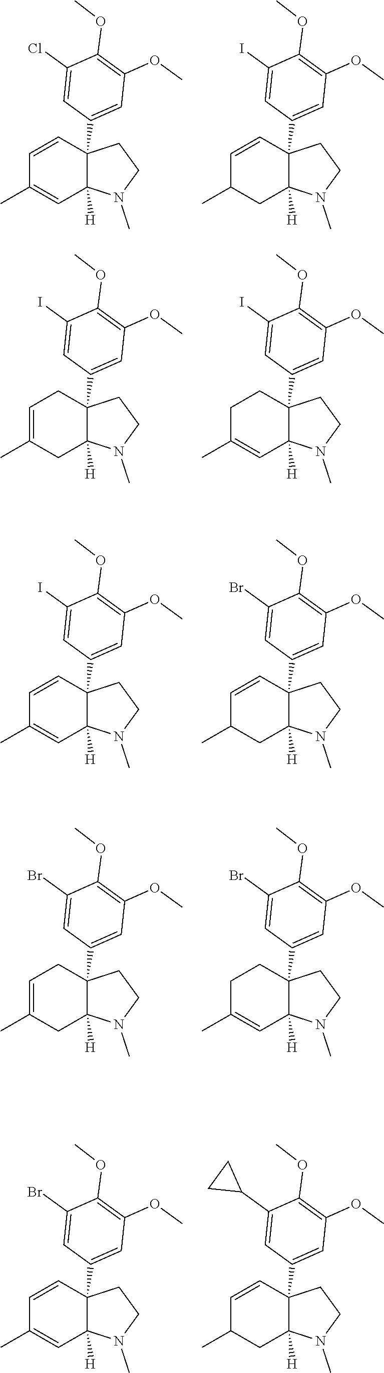

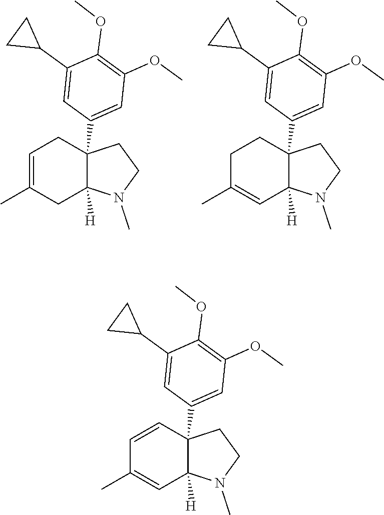

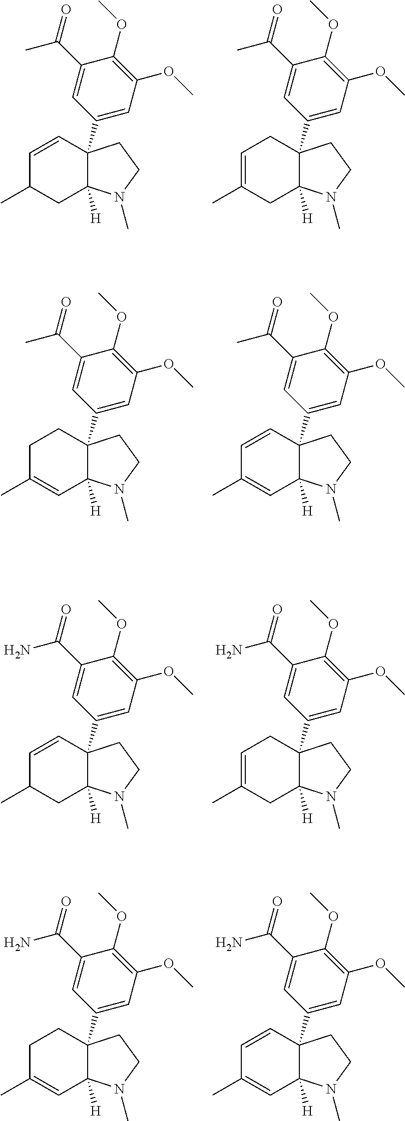

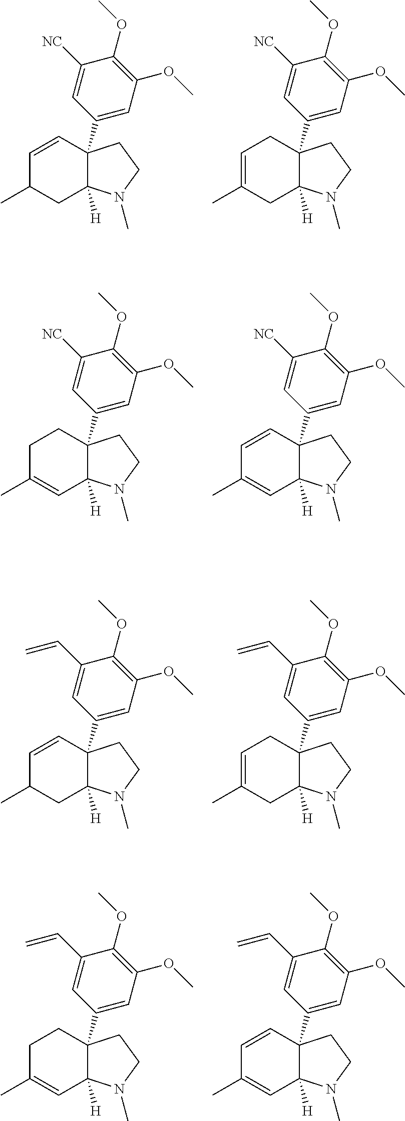

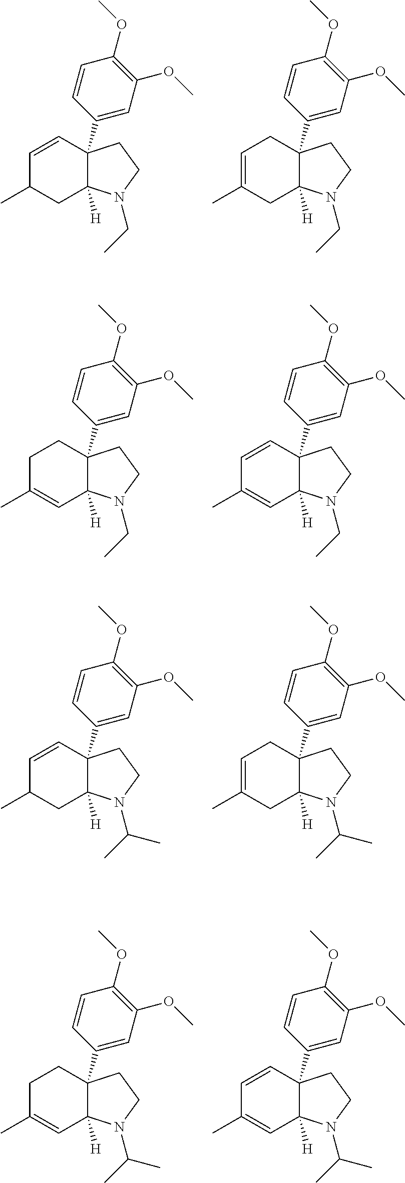

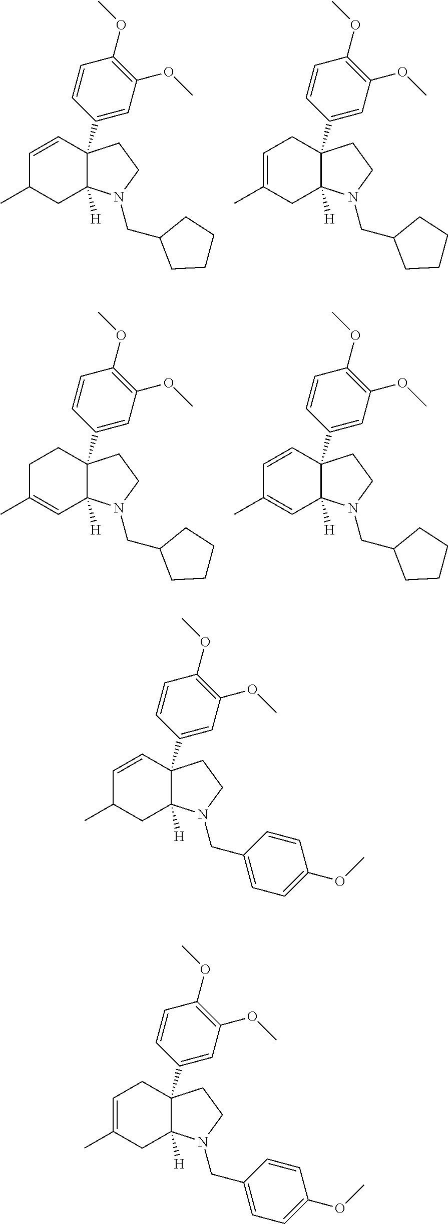

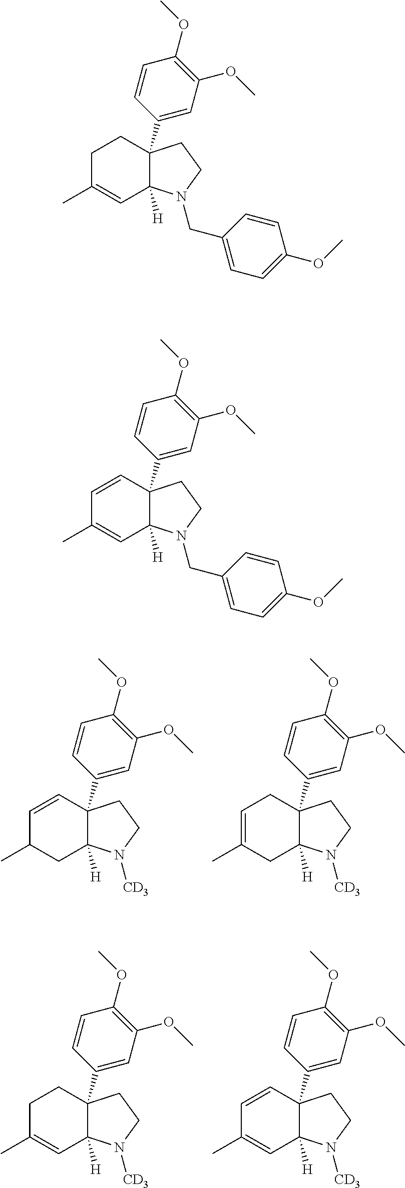

In certain embodiments, the present application is directed to a pharmaceutical composition comprising an active pharmaceutical ingredient. In certain embodiments, the pharmaceutical composition comprises a compound as disclosed herein as the active pharmaceutical ingredient (API) and a pharmaceutically acceptable carrier comprising one or more excipients. In some embodiments, the pharmaceutical composition optionally further comprises an additional therapeutic compound (i.e., agent) with the pharmaceutically acceptable carrier. The pharmaceutical composition can be a medicament.

Pharmaceutically acceptable carriers include those known in the art. The choice of a pharmaceutically acceptable carrier can depend, for example, on the desired route of administration of the composition. A pharmaceutical composition (preparation) can be administered to a subject by any of a number of routes of administration including, for example, parenteral administration (e.g., intravenously, subcutaneously, or intramuscularly), oral administration (for example, tablets, and capsules); absorption through the oral mucosa (e.g., sublingually) or transdermally (for example as a patch applied to the skin) or topically (for example, as a cream, ointment or spray applied to the skin).

In some embodiments, pharmaceutical compositions comprising compounds of a compound of Formula (I), Formula (II-A), Formula (II-B), Formula (II-C), or Formula (II-D), or pharmaceutically acceptable salts thereof can be formulated for oral administration. For example, a compound provided herein can be combined with suitable compendial excipients to form an oral unit dosage form, such as a capsule or tablet, containing a target dose of a compound of a compound of Formula (I), Formula (II-A), Formula (II-B), Formula (II-C), or Formula (II-D). The drug product can be prepared by first manufacturing the compound of a compound of Formula (I), Formula (II-A), Formula (II-B), Formula (II-C), Formula (II-D) Formula (III) or Formula (IV) as an active pharmaceutical ingredient (API), followed by roller compaction/milling with intragranular excipients and blending with extra granular excipients. A drug product can contain the selected compound of a compound of Formula (I), Formula (II-A), Formula (II-B), Formula (II-C), Formula (II-D) Formula (III) or Formula (IV) as the API and excipient components in a tablet in a desired dosage strength. The blended material can be compressed to form tablets and then film coated. The excipients can be selected from materials appropriate for inclusion in a pharmaceutical composition for an intended purpose and route of delivery including providing desired manufacturing and stability properties and/or desired in vivo characteristics or other properties to the pharmaceutical composition. In some embodiments, the pharmaceutical composition can include a compound of a compound of Formula (I), Formula (II-A), Formula (II-B), Formula (II-C), Formula (II-D) Formula (III) or Formula (IV) as the API in combination with a filler (e.g., a form of microcrystalline cellulose), a dry binder or disintegrant (e.g., a cross-linked polymer), a glidant (e.g., colloidal silicon dioxide) and/or a lubricant (e.g., magnesium stearate). In some embodiments, the pharmaceutical composition can comprise a material such as an extended release or disintegrant involved in carrying or transporting the API pharmaceutical agent from one organ, or portion of the body, to another organ, or portion of the body of a subject, including materials to desirable control the absorption of the API in the intestine.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be the amount of the compound which produces a therapeutic effect. For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

To prepare solid dosage forms for oral administration, the active ingredient is mixed with one or more pharmaceutically acceptable carriers, such as sodium citrate or dicalcium phosphate, and/or any of the following: (1) fillers or extenders, (2) binders, (3) humectants, (4) disintegrating agents, (5) solution retarding agents, (6) absorption accelerators. (7) wetting agents, (8) absorbents. (9) lubricants. (10) complexing agents, and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using suitable excipients. The pharmaceutical compositions according to the present invention may contain conventional pharmaceutical carriers and/or auxiliary agents. In some embodiments, the pharmaceutical compositions according to the present invention may contain conventional carrier agents including a binder, a lubricant and/or a glidant selected from those products and materials generally used in pharmaceutical industry for preparation of pharmaceutical compositions for an intended route of administration.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

Liquid dosage forms useful for oral administration include pharmaceutically acceptable carriers and the active ingredient provided as a solid form for reconstitution prior to administration or as a liquid (e.g., solutions, suspensions, or emulsions). In addition to the active ingredient, liquid dosage forms may contain inert diluents commonly used in the art. For example, formulations of pharmaceutically acceptable compositions for injection can include aqueous solutions such as water or physiologically buffered saline or other solvents or vehicles suitable for the intended route of administration. In some embodiments, the pharmaceutical composition is formulated for parenteral administration.

The therapeutically effective amount of a pharmaceutical composition can be determined by human clinical trials to determine the safe and effective dose for a patient with a relevant diagnosis. It is generally understood that the effective amount of the compound may vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the pharmaceutical composition at a dose and dose interval determined to be safe and effective for the patient.

The present disclosure includes the use of pharmaceutically acceptable salts of compounds of the invention in the compositions and methods of the present invention. Pharmaceutically acceptable salts include, for example, acid-addition salts and base addition salts. The acid that is added to a compound to form an acid-addition salt can be an organic acid or an inorganic acid. A base that is added to a compound to form a base addition salt can be an organic base or an inorganic base. In some embodiments, a pharmaceutically acceptable salt is a metal salt, in some embodiments, a pharmaceutically acceptable salt is an ammonium salt. For example, a pharmaceutically acceptable acid addition salt can exist as various solvates, such as with water, methanol, ethanol, dimethylformamide, and the like. Mixtures of such solvates can also be prepared. The source of such solvates can be from the solvent of crystallization, inherent in the solvent of preparation or crystallization, or adventitious to such solvent.

In some embodiments, a compound of a compound of Formula (I), Formula (II-A), Formula (II-B), Formula (II-C), Formula (II-D) Formula (III) or Formula (IV) can provide additional beneficial properties. For example, the compounds described herein may provide beneficial therapeutic properties while minimizing emesis. For example, compounds of a compound of Formula (I), Formula (II-A), Formula (II-B), Formula (II-C), Formula (II-D) Formula (III) or Formula (IV) may have improved metabolic stability and activity for inhibiting SERT. In some embodiments, the compounds of a compound of Formula (I), Formula (II-A), Formula (II-B), Formula (II-C), Formula (II-D) Formula (III) or Formula (IV) described herein inhibit SERT and have a desired metabolic stability and/or other desired ADME properties. Furthermore, compounds that have a high brain exposure with brain:plasma ratios (expressed as Kp)>0.3 and ideally >0.7 are most desirable. In some embodiments, a compound of a compound of Formula (I), Formula (II-A), Formula (II-B), Formula (II-C), Formula (II-D) Formula (III) or Formula (IV) can inhibit SERT with an IC₅₀ of less than about 1 micromolar in the assay of Example A1.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject.” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl that may be substituted, as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups, C₁-C₁₀ branched-chain alkyl groups, cycloalkyl (alicyclic) groups, alkyl-substituted cycloalkyl groups, and cycloalkyl-substituted alkyl groups. Preferably, the “alkyl” group refers to C₁-C₇ straight-chain alkyl groups or C₁-C₇ branched-chain alkyl groups. Most preferably, the “alkyl” group refers to C₁-C₃ straight-chain alkyl groups or C₁-C₃ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “haloalkyl” refers to an alkyl group substituted with at least one hydrogen atom on a carbon replaced by a halogen. Illustrative halogens include fluoro, chloro, bromo, and iodo. Illustrative haloalkyl groups include trifluoromethyl and 2,2,2-trifluoroethyl, etc.

The term “alkoxyalkyl” refers to an alkyl group substituted with an alkoxy group and may be represented by the general formula alkyl-O-alkyl.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. Co alkyl indicates a hydrogen where the group is in a terminal position, a bond if internal. A C₁₋₆ alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “amide”, as used herein, refers to a group

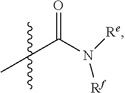

wherein R^{e} and R^{f} each independently represent a hydrogen or hydrocarbyl group, or R^{e} and R^{f} taken together with the N atom to which they are attached complete a heterocycle having from 4 to 8 atoms in the ring structure.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Preferably, the “alkoxy” group refers to C₁-C₇ straight-chain alkoxy groups or C₁-C₇ branched-chain alkoxy groups. Representative alkoxy groups include methoxy, ethoxy, propoxy, tert-butoxy and the like.

The terms “amine” and “amino” are art-recognized and refer to both unsubstituted and substituted amines and salts thereof, e.g., a moiety that can be represented by

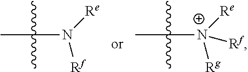

wherein R^{e}, R^{f}, and R^{g}, each independently represent a hydrogen or a hydrocarbyl group, or R^{e} and R^{f} taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein includes substituted or unsubstituted single-ring aromatic groups in which each atom of the ring is carbon. Preferably the ring is a 5- to 7-membered ring, more preferably a 6-membered ring, for example a phenyl. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

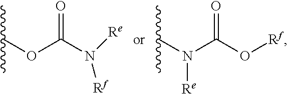

wherein R^{e} and R^{f} independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group-OCO₂—.

The term “carboxy”, as used herein, refers to a group represented by the formula-CO₂H.

The term “ester”, as used herein, refers to a group —C(O)OR⁹ wherein R⁹ represents a hydrocarbyl group.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains six or fewer carbon atoms, preferably four or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

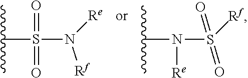

wherein R^{e} and R^{f} independently represent hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR^{e} or —SC(O)R^{e}, wherein R^{e} represents a hydrocarbyl.

The term “thioether”, as used herein, is equivalent to an ether, wherein the oxygen is replaced with a sulfur.

The term “urea” is art-recognized and may be represented by the general formula

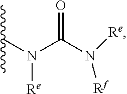

wherein R^{e} and R^{f} independently represent hydrogen or a hydrocarbyl.

The term “modulate” as used herein includes the inhibition or suppression of a function or activity (such as cell proliferation) as well as the enhancement of a function or activity.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula (I), Formula (II-A), Formula (II-B), Formula (II-C), Formula (II-D), Formula (III) or Formula (IV). Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula (I), Formula (II-A), Formula (II-B), Formula (II-C), Formula (II-D), Formula (III) or Formula (IV) are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula (I), Formula (II-A), Formula (II-B), Formula (II-C), Formula (II-D), Formula (III) or Formula (IV) for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula (I), Formula (II-A), Formula (II-B), Formula (II-C), Formula (II-D), Formula (III) or Formula (IV) or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Many of the compounds useful in the methods and compositions of this disclosure have at least one stereogenic center in their structure. This stereogenic center may be present in an R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of Formula (I), Formula (II-A), Formula (II-B), Formula (II-C), or Formula (II-D)). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs include using ester or phosphoramidate as biologically labile or cleavable (protecting) groups. The prodrugs of this disclosure are metabolized to produce a compound of Formula (I), Formula (II-A), Formula (II-B), Formula (II-C), or Formula (II-D). The present disclosure includes within its scope prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

Methods of using compounds disclosed herein are also provided. The disclosure also includes pharmaceutical compositions comprising one or more SERT inhibiting compounds, as described herein, or a pharmaceutically acceptable salt thereof, and a pharmaceutically acceptable excipient. In some embodiments, pharmaceutical compositions reported herein can be provided in a unit dosage form (e.g., capsule, tablet or the like). Pharmaceutical compositions comprising a compound of Formula (I), Formula (II-A), Formula (II-B), Formula (II-C), or Formula (II-D), can be provided in an oral dosage form such as a capsule or tablet. The oral dosage form optionally comprises one or more fillers, disintegrants, lubricants, glidants, anti-adherents and/or anti-statics. In some embodiments, an oral dosage form is prepared via dry blending. In some embodiments, an oral dosage form is a tablet and is prepared via dry granulation. For example, a SERT inhibitor compound of Formula (I), Formula (II-A), Formula (II-B), Formula (II-C), Formula (II-D) Formula (III) or Formula (IV) of the present disclosure can be formulated as a test article for evaluation in animal models and (if appropriate) subsequent human clinical trials to determine the dose at a therapeutically effective dose and dose frequency for humans. The pharmaceutical compositions may be orally administered in any orally acceptable dosage form. Accordingly, a patient and/or subject can be selected for treatment using a compound described herein by first evaluating the patient and/or subject to determine whether the subject is in need of inhibition of SERT, and if the subject is determined to be in need of inhibition of SERT, then administering to the subject a pharmaceutical composition comprising one or more compounds described herein, or pharmaceutically acceptable salts thereof.

The compounds described herein may be administered to treat CNS disorders and/or inflammatory conditions. Exemplary CNS disorders include generalized anxiety, acute anxiety and panic attacks, social anxiety, panic disorders, major depressive disorder, cognitive disorders, including Alzheimer's disease and other neurodegenerative disorders, neurodevelopmental disorders, schizophrenia, bipolar disorder, obsessive-compulsive disorder, multiple sclerosis, attention deficit-hyperactivity disorder, Bulimia nervosa, Huntington's disease, stroke, autism, and premenstrual dysphoric disorder. Exemplary inflammatory conditions include chronic obstructive pulmonary disease (COPD), asthma, and rheumatoid arthritis.

In some embodiments, methods of treating a patient suffering from a disease comprise administering to a patient a composition comprising a compound disclosed herein for the treatment or prevention of a mental health disorder. In some embodiments, methods of treating a patient suffering from a disease comprise administering to a patient a composition comprising a compound disclosed herein for the treatment or prevention of a diagnosed condition selected from anxiety and depression. In some embodiments, the compound disclosed herein is administered to the patient in a unit dose. In some embodiments, a method comprises the administration to a patient in need thereof of a therapeutically effective amount of a compound of Formula (I), Formula (II-A), Formula (II-B), Formula (II-C), or Formula (II-D), for the treatment of a disease selected from the group consisting of mild to moderate depression and major depressive episodes. In some embodiments, a method comprises the administration to a patient in need thereof of a therapeutically effective amount of a compound of Formula (I), Formula (II-A), Formula (II-B), Formula (II-C), or Formula (II-D), for the treatment of anxiety. In some embodiments, a method comprises the administration to a patient in need thereof of a therapeutically effective amount of a compound of Formula (I), Formula (II-A), Formula (II-B), Formula (II-C), or Formula (II-D), for the treatment of depression. In some embodiments, a method comprises the administration to a patient in need thereof of a therapeutically effective amount of a compound of Formula (I), Formula (II-A), Formula (II-B), Formula (II-C), or Formula (II-D), for the treatment of a condition selected from the group consisting of: anxiety associated with depression, anxiety with depression, mixed anxiety and depressive disorder. In some embodiments, a method comprises the administration to a patient in need thereof of a therapeutically effective amount of a compound of Formula (I), Formula (II-A), Formula (II-B), Formula (II-C), or Formula (II-D), for the treatment of anxiety and hysteria or anxiety and depression.

Unless otherwise indicated in the tables of compounds herein, the abbreviation RAC or rac indicates a racemic mixture, and DIAST indicates a specific diastereomer. In illustrative embodiments, although a compound may be depicted with [symbol] or [symbol] bonds, such a depiction may be denoting relative stereochemistry based on elution peaks from a chiral separation.

In some embodiments, this disclosure provides one or more of the following embodiments of the invention, or variations thereof:
1. A compound of formula (I), or a pharmaceutically acceptable salt thereof:

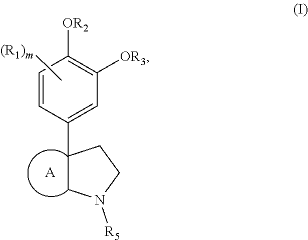

wherein
ring A is

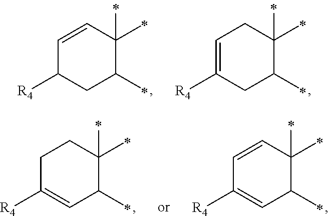

wherein * denotes the attachment points of ring A to the compound of formula (I), and wherein
each R₁ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O) NR^{a}R^{b}, —CN, nitro, or —P(O)OR^{a}OR^{b};
m is 0, 1, 2, or 3;
each of R₂ and R₃ is independently H, alkyl, alkenyl, alkynyl, cycloalkyl, heterocycloalkyl, or aryl; wherein each hydrogen atom in alkyl cycloalkyl, alkenyl, alkynyl, and aryl is optionally substituted by halo, deuterium, cycloalkyl, aryl, or OR^{a}; or
R₂ and R₃ together with the atoms to which they are attached combine to form heterocyclyl or heteroaryl, wherein each hydrogen atom in heterocyclyl and heteroaryl is optionally substituted by halo or OR^{a};
R^{a} and R^{b} are independently H, alkyl (e.g., methyl), alkenyl, cycloalkyl, heterocycloalkyl, aryl, or heteroaryl; or if an instance of R₁ is —NR^{a}R^{b}, then R^{a} and R^{b} may combine with the nitrogen atom to which they are attached to form heterocycloalkyl or heteroaryl;
R₄ is H, C₁₋₄ alkyl or C₁₋₆ haloalkyl; and
R₅ is C₁₋₄ alkyl.
2. The compound of embodiment 1, wherein ring A is

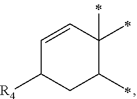

3. The compound of embodiment 1, wherein ring A is

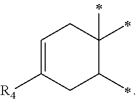

4. The compound of embodiment 1, wherein ring A is

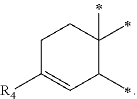

5. The compound of embodiment 1, wherein ring A is

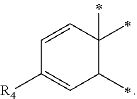

6. The compound of embodiment 1, wherein the compound is a compound of Formula (II-A),

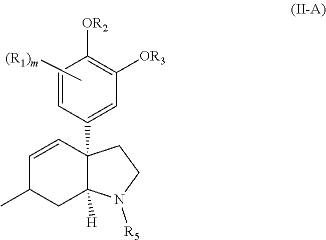

or a pharmaceutically acceptable salt thereof.
7. The compound of embodiment 1, wherein the compound is a compound of Formula (II-B),

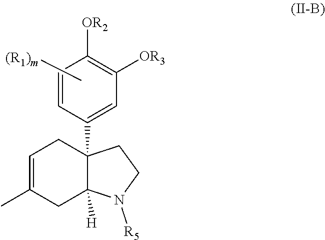

or a pharmaceutically acceptable salt thereof.
8. The compound of embodiment 1, wherein the compound is a compound of Formula (II-C),

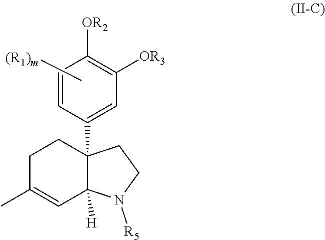

or a pharmaceutically acceptable salt thereof.
9. The compound of embodiment 1, wherein the compound is a compound of Formula (II-D),

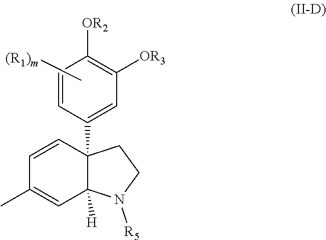

or a pharmaceutically acceptable salt thereof.
10. The compound of any one of embodiments 1-9, or a pharmaceutically acceptable salt thereof, wherein:
   a. each R₁ is independently hydrogen, halo, C₁₋₄ alkyl, or C₃₋₆ cycloalkyl; wherein each hydrogen atom in C₁₋₄ alkyl, or C₃₋₆ cycloalkyl is optionally substituted by halo or C₁₋₄ alkyl;
   b. m is 0 or 1;
   c. each of R₂ and R₃ is independently hydrogen, halo, C₁₋₄ alkyl, or C₃₋₆ cycloalkyl; wherein each hydrogen atom in C₁₋₄ alkyl, or C₃₋₆ cycloalkyl is optionally substituted by halo or C₁₋₄ alkyl;
   d. R₄ is H, C₁₋₄ alkyl or C₁₋₄ haloalkyl; and
   e. R₅ is C₁₋₂ alkyl.
11. The compound of embodiment 10, or a pharmaceutically acceptable salt thereof, wherein:
   a. each R₁ is independently hydrogen, halo, methyl, ethyl, or cyclopropyl; wherein each hydrogen atom in methyl, ethyl, or cyclopropyl is optionally substituted by halo, methyl or halomethyl;
   b. m is 0 or 1;
   c. each of R₂ and R₃ is independently hydrogen, halo, methyl, or halomethyl; wherein each hydrogen atom in methyl, ethyl, or cyclopropyl is optionally substituted by halo, methyl or halomethyl;
   d. R₄ is H, C₁₋₄ alkyl, or C₁₋₄ haloalkyl; and
   e. R₅ is methyl.
12. The compound of embodiment 11, or a pharmaceutically acceptable salt thereof, wherein:
   a. each R₁ is independently hydrogen, fluoro, methyl optionally substituted with one or more fluoro, or cyclopropyl;
   b. m is 0 or 1;
   c. each of R₂ and R₃ is independently hydrogen, fluoro, or methyl optionally substituted with one or more fluoro;
   d. R₄ is H, C₁₋₄ alkyl or C₁₋₄ haloalkyl; and
   e. R₅ is methyl.
13. The compound of any one of embodiments 1-12, wherein R₁ is halo.
14. The compound of any one of embodiments 1-12, wherein R₁ is fluoro.
15. The compound of any one of embodiments 1-12, wherein R₁ is cyclopropyl.
16. The compound of any one of embodiments 1-15, wherein m is 0 or 1.
17. The compound of any one of embodiments 1-16, wherein R₂ is halomethyl.
18. The compound of embodiment 17, wherein R₂ is —CHF₂ or —CF₃.
19. The compound of any one of embodiments 1-18, wherein R₅ is methyl.
20. The compound of any one of embodiments 1-19, wherein R₃ is methyl.
21. The compound of any one of embodiments 1-20, wherein R₄ is methyl.
22. The compound of embodiment 1, wherein the compound is

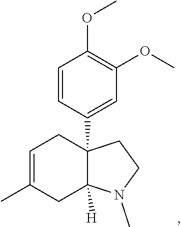

or a pharmaceutically acceptable salt thereof.
23. The compound of embodiment 1, wherein the compound is

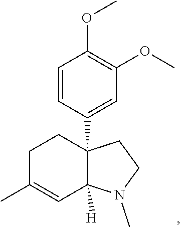

or a pharmaceutically acceptable salt thereof.
24. The compound of embodiment 1, wherein the compound is

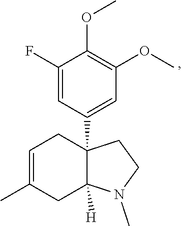

or a pharmaceutically acceptable salt thereof.
25. The compound of embodiment 1, wherein the compound is

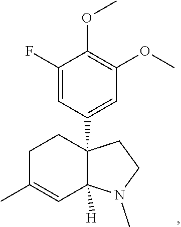

or a pharmaceutically acceptable salt thereof.
26. The compound of embodiment 1, wherein the compound is

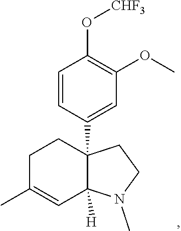

or a pharmaceutically acceptable salt thereof.
27. The compound of embodiment 1, wherein the compound is

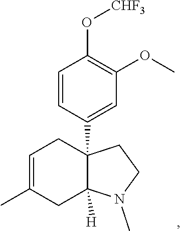

or a pharmaceutically acceptable salt thereof.
28. The compound of embodiment 1, wherein the compound is

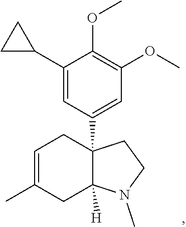 [SMILES: COc1cc([C@@]23CC=C(C)C[C@@H]2N(C)CC3)cc(C2CC2)c1OC]

or a pharmaceutically acceptable salt thereof.
29. The compound of embodiment 1, wherein the compound is

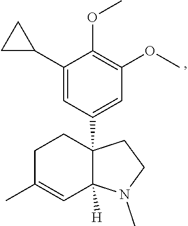 [SMILES: COc1cc([C@@]23CCC(C)=C[C@@H]2N(C)CC3)cc(C2CC2)c1OC]

or a pharmaceutically acceptable salt thereof.
30. The compound of any one of embodiments 1-9, wherein R₂ and R₃ together with the atoms to which they are attached combine to form heterocyclyl or heteroaryl, wherein each hydrogen atom in heterocyclyl and heteroaryl is optionally substituted by halo or OR^{a};
R₄ is H, C₁₋₄ alkyl or C₁₋₆ haloalkyl; and
R₅ is C₁₋₄ alkyl.
31. The compound of embodiment 30, or a pharmaceutically acceptable salt thereof, wherein:
   a. each R₁ is independently hydrogen, halo, C₁₋₄ alkyl, or C₃₋₆ cycloalkyl;
   wherein each hydrogen atom in C₁₋₄ alkyl, or C₃₋₆ cycloalkyl is optionally substituted by halo or C₁₋₄ alkyl;
   b. m is 0 or 1;
   c. R₂ and R₃ together with the atoms to which they are attached combine to form a 5-6 membered heterocyclyl, wherein each hydrogen atom in heterocyclyl is optionally substituted by halo, C₁₋₄ alkyl or C₁₋₄ haloalkyl;
   d. R₄ is H, C₁₋₄ alkyl or C₁₋₄ haloalkyl; and
   e. R₅ is C₁-2 alkyl.
32. The compound of embodiment 31, or a pharmaceutically acceptable salt thereof, wherein:
   a. each R₁ is independently hydrogen, halo, methyl, ethyl, or cyclopropyl;
   wherein each hydrogen atom in methyl, ethyl, or cyclopropyl is optionally substituted by halo, methyl or halomethyl;
   b. m is 0 or 1;
   c. R₂ and R₃ together with the atoms to which they are attached combine to form a 5-6 membered heterocyclyl, wherein each hydrogen atom in heterocyclyl is optionally substituted by halo, C₁₋₄ alkyl or C₁₋₄ haloalkyl;
   d. R₄ is H, C₁₋₄ alkyl or C₁₋₄ haloalkyl; and
   e. R₅ is methyl.
33. The compound of embodiment 32, or a pharmaceutically acceptable salt thereof, wherein:
   a. each R₁ is independently hydrogen, fluoro, methyl optionally substituted with one or more fluoro, or cyclopropyl;
   b. m is 0 or 1;
   c. R₂ and R₃ together with the atoms to which they are attached combine to form a 5-6 membered heterocyclyl, wherein each hydrogen atom in heterocyclyl is optionally substituted by fluoro or methyl optionally substituted with one or more fluoro;
   d. R₄ is H, C₁₋₄ alkyl or C₁₋₄ haloalkyl; and
   e. R₅ is methyl.
34. The compound of any one of embodiments 30-33, wherein R₁ is halo.
35. The compound of any one of embodiments 30-33, wherein R₁ is fluoro.
36. The compound of any one of embodiments 30-33, wherein R₁ is cyclopropyl.
37. The compound of any one of embodiments 30-33, wherein m is 0 or 1.
38. The compound of any one of embodiments 30-33, wherein R₂ and R₃ together with the atoms to which they are attached combine to form a 5-membered heterocyclyl.
39. The compound of any one of embodiments 30-38, wherein R₂ and R₃ together with the atoms to which they are attached combine to form an unsubstituted 5-membered heterocyclyl.
40. The compound of any one of embodiments 30-38, wherein R₂ and R₃ together with the atoms to which they are attached combine to form a 6-membered heterocyclyl.
41. The compound of any one of embodiments 30-38, wherein R₂ and R₃ together with the atoms to which they are attached combine to form an unsubstituted 6-membered heterocyclyl.
42. The compound of any one of embodiments 30-41, wherein R₅ is methyl.
43. The compound of any one of embodiments 30-42, wherein R₄ is methyl.
44. The compound of embodiment 30, wherein the compound is

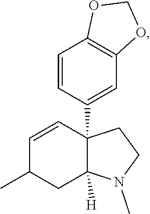 [SMILES: CC1C=C[C@@]2(c3ccc4c(c3)OCO4)CCN(C)[C@H]2C1]

or a pharmaceutically acceptable salt thereof.
45. The compound of embodiment 30, wherein the compound is

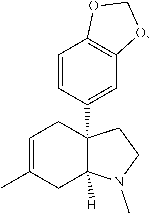 [SMILES: CC1=CC[C@@]2(c3ccc4c(c3)OCO4)CCN(C)[C@H]2C1]

or a pharmaceutically acceptable salt thereof.
46. The compound of embodiment 30, wherein the compound is

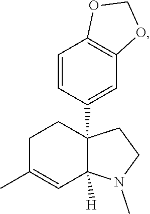 [SMILES: CC1=C[C@@H]2N(C)CC[C@]2(c2ccc3c(c2)OCO3)CC1]

or a pharmaceutically acceptable salt thereof.
47. The compound of embodiment 30, wherein the compound is

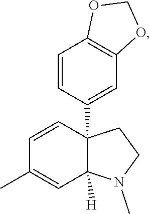 [SMILES: CC1=C[C@@H]2N(C)CC[C@]2(c2ccc3c(c2)OCO3)C=C1]

or a pharmaceutically acceptable salt thereof.
48. The compound of embodiment 1, wherein the compound is

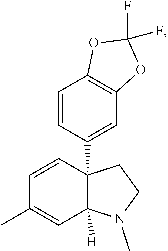 [SMILES: CC1=C[C@@H]2N(C)CC[C@]2(c2ccc3c(c2)OC(F)(F)O3)C=C1]

or a pharmaceutically acceptable salt thereof.
49. A pharmaceutical composition, comprising a compound of any one of embodiments 1-48, or a pharmaceutically acceptable salt thereof; and a pharmaceutically acceptable excipient.
50. A method of treating a mental health disorder, comprising administering to a mammal in need thereof an effective amount of a compound according to any one of embodiments 1-48 or a pharmaceutically acceptable salt thereof, or the pharmaceutical composition of embodiment 49.
51. The method of embodiment 50, wherein the mental health disorder is anxiety, stress, or depression.
52. The method of embodiment 50, wherein the mental health disorder is anxiety.
53. The method of embodiment 50, wherein the mental health disorder is stress.
54. The method of embodiment 50, wherein the mental health disorder is depression.
55. The method of any one of embodiments 50-54, wherein the mammal is a human.
56. The compound of embodiment 1, wherein the compound is

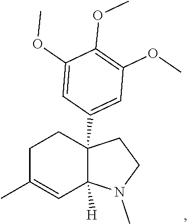

or a pharmaceutically acceptable salt thereof.
57. The compound of embodiment 1, wherein the compound is

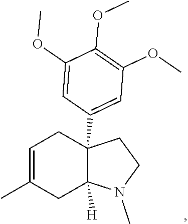

or a pharmaceutically acceptable salt thereof.
58. The compound of embodiment 1, wherein the compound is

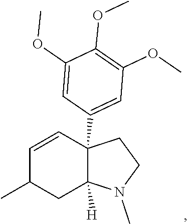

or a pharmaceutically acceptable salt thereof.

59. The compound of embodiment 1, wherein the compound is

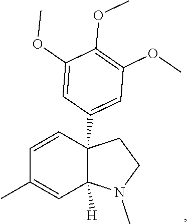

or a pharmaceutically acceptable salt thereof.
60. The compound of embodiment 30, wherein the compound is

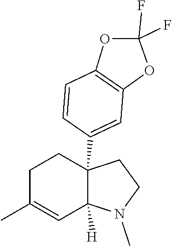 [SMILES: CC1=C[C@@H]2N(C)CC[C@]2(c2ccc3c(c2)OC(F)(F)O3)CC1]

or a pharmaceutically acceptable salt thereof.
61. The compound of embodiment 30, wherein the compound is

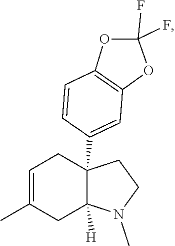 [SMILES: CC1=CC[C@@]2(c3ccc4c(c3)OC(F)(F)O4)CCN(C)[C@H]2C1]

or a pharmaceutically acceptable salt thereof.
62. The compound of embodiment 30, wherein the compound is

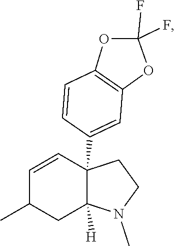 [SMILES: CC1C=C[C@@]2(c3ccc4c(c3)OC(F)(F)O4)CCN(C)[C@H]2C1]

or a pharmaceutically acceptable salt thereof.

### Examples

Certain compounds in the following examples are labeled using the MDL enhanced stereo representation. For example, the label “ABS” denotes the absolute stereochemistry at a particular stereocenter. The label “or n” or “orn” where n is an integer (e.g., “or1”), denotes a stereoisomer that has either the stereochemistry as drawn or is the epimer at that particular stereocenter. The label “and n” or “& n,” where n is an integer (e.g., “and 1” or “& 1”) represents a mixture of two epimers at the stereocenter, i.e., the structure as drawn and the epimer in which the stereogenic center has the opposite configuration (e.g., a racemic mixture).

LC/MS spectra were obtained using Agilent 1200\G1956A or SHIMADZU LCMS-2020. Standard LC/MS conditions were as follows (running time 1.55 min):

Acidic condition: Mobile Phase A: 0.0375% TFA in water (v/v). Mobile Phase B: 0.01875% TFA in acetonitrile (v/v); Column: Kinetex EVO C18 30*2.1 mm, 5 μm.

Basic condition: Mobile Phase A: 0.025% NH₃·H₂O in water (v/v). Mobile Phase B: Acetonitrile; Column: Kinetex EVO C18 2.1×30 mm, 5 μm.

5-95AB_0.8 min

| Instrument | | SHIMADZU LCMS-2020; | | |
|---|---|---|---|---|
| Software | | LabSolution Version 5.97SP1 | | |
| HPLC | Column | Kinetext ® EVO C₁₈ 2.1 × 30 mm | | |
| | | 5 um | | |
| | Mobile | A: 0.0375% TFA in water | | |
| | Phase | (v/v) | | |
| | | B: 0.01875% TFA in | | |
| | | Acetonitrile (v/v) | | |
| | | Time | B | Flow |
| | Gradient | (min) | (%) | (mL/min) |
| | | 0.00 | 5.0 | 2.0 |
| | | 0.60 | 95.0 | 2.0 |
| | | 0.78 | 95.0 | 2.0 |
| | | 0.79 | 5.0 | 2.0 |
| | | 0.80 | 5.0 | 2.0 |

| | | |
|---|---|---|
| | Column | 50° C. |
| | Temp | |
| | Detector | PDA (220 nm & 254 nm) |
| MS | Ionization | ESI |
| | source | |
| | Drying Gas | N₂ |
| | Drying Gas | 15 (L/min) |
| | Flow | |
| | DL Voltage | 120(V) |
| | Qarray | 20(V) |
| | DC Voltage | |
| | MS Polarity | Positive |
| | MS Mode | Scan |
| | Mass range | 100-1000 |

Table of Abbreviations

| | |
|---|---|
| Ac | Acetyl |
| ACN | Acetonitrile |
| Bn | Benzyl |
| δ | Chemical shift |
| d | Doublet |
| DCE | 1,2-Dichloroethane |
| DCM | Dichloromethane |
| DEA | Diethylamine |
| DIBAL | Diisobutylaluminium hydride |
| DMF | N,N-Dimethylformamide |
| ESI | Electrospray ionization |
| Et | Ethyl |
| FA | Formic Acid |
| HPLC | High Performance Liquid Chromatography |
| hr | Hours |
| Hz | Hertz |
| IPA | Isopropanol |
| LC-MS | Liquid chromatography - mass spectrometry |
| m | Multiplet |
| m/z | Mass to charge ratio |
| Me | Methyl |
| min | Minutes |
| NMR | Nuclear magnetic resonance |
| rac- | racemic |
| s | Singlet (NMR) or seconds (time) |
| SFC | Supercritical fluid chromatography |
| t | Triplet |
| t- | Tertiary- |
| Tf | Trifluoromethanesulfonyl |
| TFA | Trifluoroacetic acid |
| THF | Tetrahydrofuran |
| TMS | Trimethylsilyl |
| TOAB | Tetraoctylammonium bromide |
| TosMIC | Toluenesulfonylmethyl isocyanide |
| V | Volts |
| v/v | Volume per volume |
| XtalFluor- | (Diethylamino)difluorosulfonium tetrafluoroborate |

### General Synthetic Method A: Synthesis of Methylenecyclohexane and Methylcyclohexene Compounds from Corresponding Cyclohexanone Compounds

Step 1: To a solution of methyl(triphenyl)phosphonium bromide (740 mg, 2.07 mmol) in THF (6.0 mL) was added t-BuOK (1 M in THF, 2.07 mL) at 0° C. over 0.5 hours. The corresponding cyclohexanone compound (1.04 mmol) in THF (1 mL) was added to the mixture. The reaction mixture was allowed to stir at 25° C. for 16 hr and then poured into water (50 mL). The mixture was extracted with ethyl acetate (100 mL). The organic solutions were dried over sodium sulfate, filtered and concentrated. The crude product was purified by reverse-phase HPLC (0.1% FA condition).

Step 2: To a solution of methylenecyclohexane compound (696 μmol) in dioxane (10 mL) was added TfOH (1.39 mmol). The mixture was allowed to stir at 80° C. for 2 h and then filtered. The filtrate was concentrated under reduced pressure. The residue was purified by prep-HPLC to give a residue. The residue was separated by SFC to the corresponding methylcyclohexene compound.

### General Synthetic Method B: Methylenecyclohexane and Methylcyclohexene Compounds

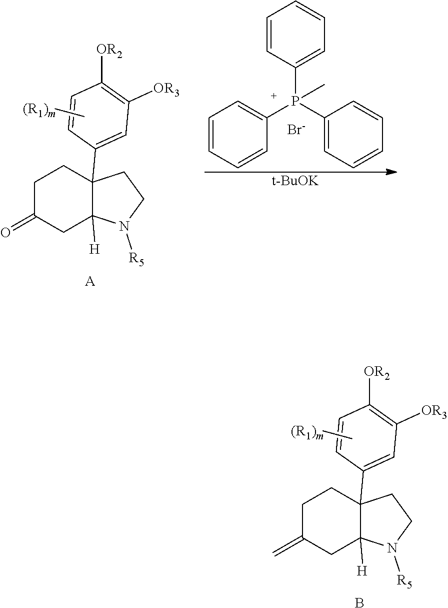

Treatment of a ketone compound such as A with a phosphonium salt, such as that formed by the treatment of methyl(triphenyl)phosphonium bromide with an alkoxide, such as t-BuOK, in a solvent, such as THF, at a low temperature, such as 0° C., is a method that can be used to prepare methylenecyclohexane compounds such as B.

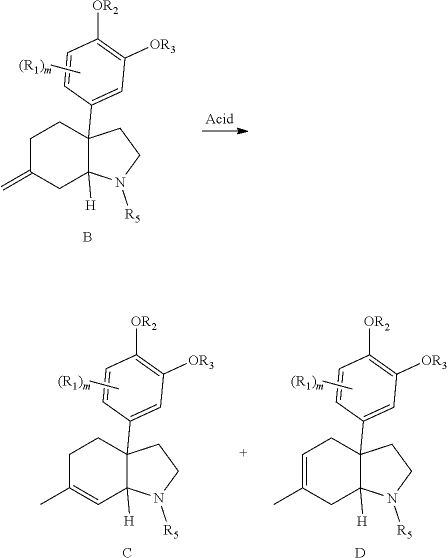

Treatment of methylenecyclohexane compounds such as B with an acid, such as trifluoromethanesulfonic acid (TfOH), in a solvent, such as dioxane, at an elevated temperature, such as 80° C., is a method that could be used to prepare methylcyclohexene compounds such as C and D.

### General Synthetic Method C

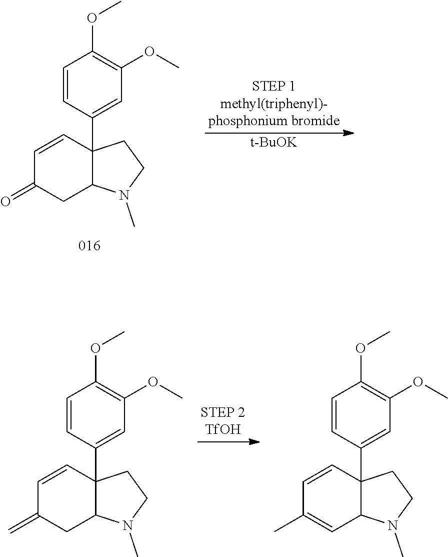

Step 1: To a solution of methyl(triphenyl)phosphonium bromide 2.07 mmol) in THF (6.0 mL) was added t-BuOK (1 M in THF, 2.07 mL) at 0° C. over 0.5 hours. 016 (1.04 mmol) in THF (1 mL) was added to the mixture. The reaction mixture was allowed to stir at 25° C. for 16 hr and then poured into water (50 mL). The mixture was extracted with ethyl acetate (100 mL). The organic solutions were dried over sodium sulfate, filtered and concentrated. The crude product was purified by reversed-phase HPLC (0.1% FA condition).

Step 2: To a solution of 3a-(3,4-dimethoxyphenyl)-1-methyl-6-methylene-2,3,3a,6,7,7a-hexahydro-1H-indole (696 μmol) in dioxane (10 mL) was added TfOH (1.39 mmol). The mixture was allowed to stir at 80° C. for 2 h and then filtered. The filtrate was concentrated under reduced pressure. The residue was purified by prep-HPLC.

### Example 1: Synthesis of (+/−)-Mesembrenone (016) (+/−)-Mesembrine (022)

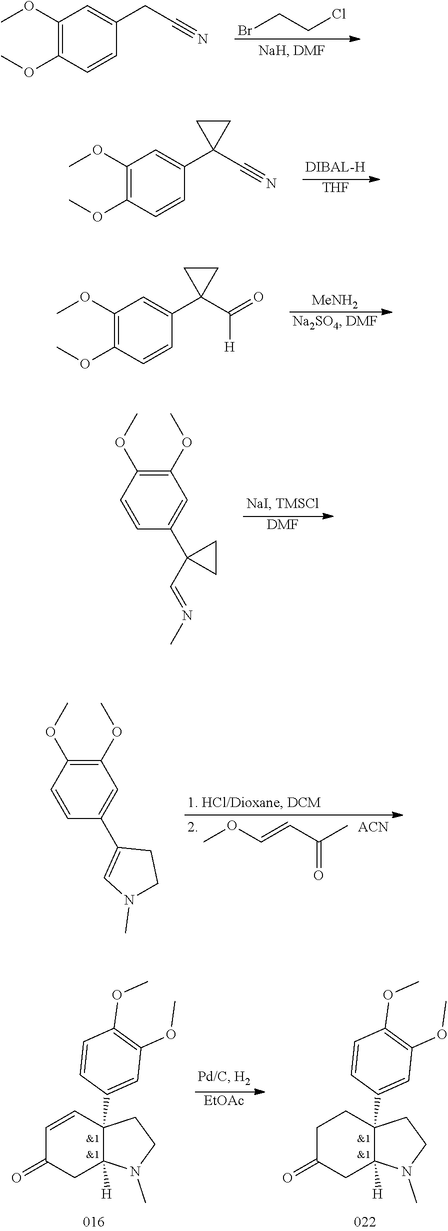

### Step 1: Synthesis of 1-(3,4-dimethoxyphenyl)cyclopropane-1-carbonitrile

To a solution of 2-(3,4-dimethoxyphenyl)acetonitrile (20 g, 112 mmol) in DMF (93 mL) was added NaH (18.0 g, 451 mmol, 60% purity) in portions. The mixture was allowed to stir at 25° C. for 20 min. 1-bromo-2-chloro-ethane (16.1 g, 112 mmol) was added, and the mixture was allowed to stir at 25° C. for 16 hr. The reaction was quenched by the addition of a MeOH/water mixture (1:1, 1000 mL) and the resulting solution was extracted with EtOAc (3×500 mL). The organic solutions were combined, washed with water (4×500 mL) and brine (1×200 mL) and dried over (Na₂SO₄). The solution was filtered and the solvent was evaporated under reduced pressure. The resulting solid was purified by column chromatography (SiO₂, Petroleum ether/EtOAc=10/1 to 3/1) to give 1-(3,4-dimethoxyphenyl)cyclopropane-1-carbonitrile (15 g, 65%) as yellow oil. ¹H NMR (400 MHz, CDCl₃) δ 6.88 (s, 1H), 6.82 (d, J=1.2 Hz, 2H), 3.91 (s, 3H), 3.88 (s, 3H), 1.68-1.65 (m, 2H), 1.35 (d, J=2.4 Hz, 2H).

### Step 2: Synthesis of 1-(3,4-dimethoxyphenyl)cyclopropane-1-carbaldehyde

To a solution of 1-(3,4-dimethoxyphenyl)cyclopropane-1-carbonitrile (11 g, 54.1 mmol) in THF (160 mL) was added DIBAL-H (1 M in toluene, 81.2 mL). The mixture was allowed to stir at 25° C. for 3 hr and then the reaction was cautiously quenched by addition of aqueous 2 M HCl. The solution was extracted with DCM (3×200 mL). The organic solutions were combined, washed with water (2×200 mL) and brine (2×200 mL), and then dried over Na₂SO₄ to give 1-(3,4-dimethoxyphenyl)cyclopropane-1-carbaldehyde (9.6 g, 85%) as yellow oil. LC-MS (ESI⁺) m/z 207.0 (M+H)⁺. ¹H NMR (400 MHz, CDCl₃) δ 9.26 (s, 1H), 6.94-6.61 (m, 3H), 3.89 (d, J=2.8 Hz, 6H), 1.61-1.52 (m, 2H), 1.42-1.37 (m, 2H)

### Step 3: Synthesis of (Z)-1-(1-(3,4-dimethoxyphenyl)cyclopropyl)-N-methylmethanamine

To a solution of 1-(3,4-dimethoxyphenyl)-cyclopropanecarbaldehyde (5.0 g, 24.2 mmol) in DCM (50 mL) was added MeNH₂ (2 M, 121 mL) and Na₂SO₄ (15.5 g, 109 mmol, 11.0 mL). The mixture was allowed to stir at 25° C. for 16 hr. The reaction mixture was filtered and the filtrate was concentrated under reduced pressure to give (Z)-1-(1-(3,4-dimethoxyphenyl)cyclopropyl)-N-methylmethanamine (5.1 g, 99%) as white solid. LC-MS (ESI⁺) m/z 219.9 (M+H)⁺; ¹H NMR (400 MHz, CDCl₃) δ 7.55 (q. J=1.2 Hz, 1H), 6.93-6.77 (m, 3H), 3.88 (d. J=7.2 Hz, 6H), 3.24 (d. J=1.6 Hz, 3H), 1.29-1.23 (m, 2H), 1.18-1.12 (m, 2H).

### Step 4: Synthesis of 4-(3,4-dimethoxyphenyl)-1-methyl-2,3-dihydro-1H-pyrrole

To a solution of (Z)-1-(1-(3,4-dimethoxyphenyl)cyclopropyl)-N-methylmethanamine (5.4 g, 24.6 mmol) in DMF (19 mL) was added NaI (366 mg, 2.44 mmol) and TMSCl (267 mg, 2.46 mmol). The mixture was allowed to stir at 90° C. for 3 hr. The reaction mixture was diluted with water (100 mL) and extracted with EtOAc (100 mL×3). The organic solutions were combined, washed with water and brine, dried over Na₂SO₄, and filtered. The filtrate was concentrated under reduced pressure to give 4-(3,4-dimethoxyphenyl)-1-methyl-2,3-dihydro-1H-pyrrole (6.25 g. 80%) as yellow oil. LC-MS (ESI⁺) m/z 220.0 (M+H)⁺. ¹H NMR (400 MHz, CDCl₃) δ 6.90-6.66 (m, 3H), 6.31 (t. J=1.6 Hz, 1H), 3.95-3.80 (m, 6H), 3.18-3.11 (m, 2H), 2.79 (dt, J=1.2, 9.0 Hz, 2H), 2.65 (s, 3H).

### Step 5: Synthesis of rac-3a-(3,4-dimethoxyphenyl)-1-methyl-1,2,3,3a,7,7a-hexahydro-6H-indol-6-one (016)

4-(3,4-Dimethoxyphenyl)-1-methyl-2,3-dihydro-1H-pyrrole (6.25 g, 28.5 mmol) was dissolved in DCM (100 mL). To this solution was added HCl (1 M in dioxane, 25 mL, 100 mmol). The mixture was evaporated to dryness and then dissolved in ACN (90 mL). To this solution was added (E)-4-methoxybut-3-en-2-one (4.28 g, 42.7 mmol). The reaction mixture was allowed to stir at 90° C. for 16 hr. The reaction mixture was filtered and the filtrate was concentrated under reduced pressure. The residue was purified by HPLC (column: Phenomenex luna C₁₈ (250*70 mm, 10 μm); mobile phase: [water (NH₄HCO₃)-ACN]; B %: 22%-52%, 20 min). The eluant was acidified with aq. HCl to give 016 (3.0 g, 30%) as a white solid. LC-MS (ESI⁺) m/z 288.3 (M+H)⁺. ¹H NMR (400 MHz, CDCl₃) δ 6.90-6.88 (m, 1H), 6.87-6.83 (m, 2H), 6.74 (dd, J=2.0, 10.1 Hz, 1H), 6.11 (d, J=10.0 Hz, 1H), 3.89 (d, J=4.0 Hz, 6H), 3.33 (dt, J=2.4, 8.8 Hz, 1H), 2.69-2.66 (m, 1H), 2.58-2.51 (m, 2H), 2.50-2.41 (m, 2H), 2.33 (s, 3H), 2.27-2.18 (m, 1H)

### Step 6: Synthesis of rac-3a-(3,4-dimethoxyphenyl)-1-methyloctahydro-6H-indol-6-one (022)

A mixture of 016 (12.0 g, 43.9 mmol) and 10% Pd/C (300 mg) in EtOAc (120 mL) was degassed and then purged with H₂ for 3 times. The mixture was allowed to stir at 25° C. for 2 hr under 15 psi H₂. The reaction mixture was filtered and the filtrate was concentrated in vacuo to give 022 (10 g, 80%) as brown oil. LC-MS (ESI⁺) m/z 290.4 (M+H)⁺. ¹H NMR (400 MHz, CDCl₃) δ 6.99-6.89 (m, 2H), 6.89-6.84 (m, 1H), 3.91 (d, J=7.6 Hz, 6H), 3.20-3.11 (m, 1H), 2.97 (t, J=3.6 Hz, 1H), 2.69-2.56 (m, 2H), 2.51-2.31 (m, 5H), 2.27-2.18 (m, 3H), 2.18-2.07 (m, 2H).

### Example 2: SFC Separation of rac-3a-(3,4-dimethoxyphenyl)-1-methyloctahydro-6H-indol-6-one (022) to give (−)-Mesembrine (001) and (+)-Mesembrine (002)

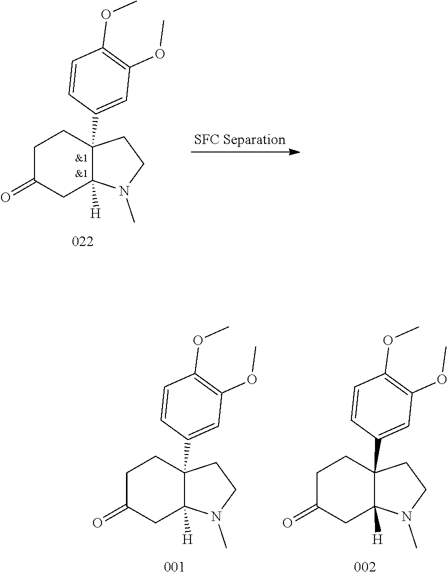 [SMILES: COc1ccc([C@@]23CCC(=O)C[C@@H]2N(C)CC3)cc1OC ; COc1ccc([C@@]23CCC(=O)C[C@@H]2N(C)CC3)cc1OC ; COc1ccc([C@]23CCC(=O)C[C@H]2N(C)CC3)cc1OC]

Compound 022 (15 g, 90% purity) was subjected to separation by SFC (column: DAICEL CHIRALCEL OD (250 mm*50 mm, 10 μm); mobile phase: [Neu-MeOH]; B %: 25%-25%, 2; 1230 min) to give 001 (peak 1, 5.4 g, free base, 36%) as yellow oil and 002 (peak 2, 5.6 g, free base, 37%) as yellow oil.
001: LC-MS (ESI⁺) m/z 290.4 (M+H)^{+ 1}H NMR (400 MHz, CDCl₃) δ 6.99-6.89 (m, 2H), 6.89-6.84 (m, 1H), 3.91 (d, J=7.6 Hz, 6H), 3.20-3.11 (m, 1H), 2.97 (t, J=3.6 Hz, 1H), 2.69-2.56 (m, 2H), 2.51-2.31 (m, 5H), 2.27-2.18 (m, 3H), 2.18-2.07 (m, 2H).
002: LC-MS (ESI⁺) m/z 290.4 (M+H)^{+ 1}H NMR (400 MHz, CDCl₃) δ 6.99-6.89 (m, 2H), 6.89-6.84 (m, 1H), 3.91 (d, J=7.6 Hz, 6H), 3.20-3.11 (m, 1H), 2.97 (t, J=3.6 Hz, 1H), 2.69-2.56 (m, 2H), 2.51-2.31 (m, 5H), 2.27-2.18 (m, 3H), 2.18-2.07 (m, 2H).

### Example 3: Synthesis of (3aS,7aS)-3a-(3,4-dimethoxyphenyl)-1,6-dimethyl-2,3,3a,4,5,7a-hexahydro-1H-indole

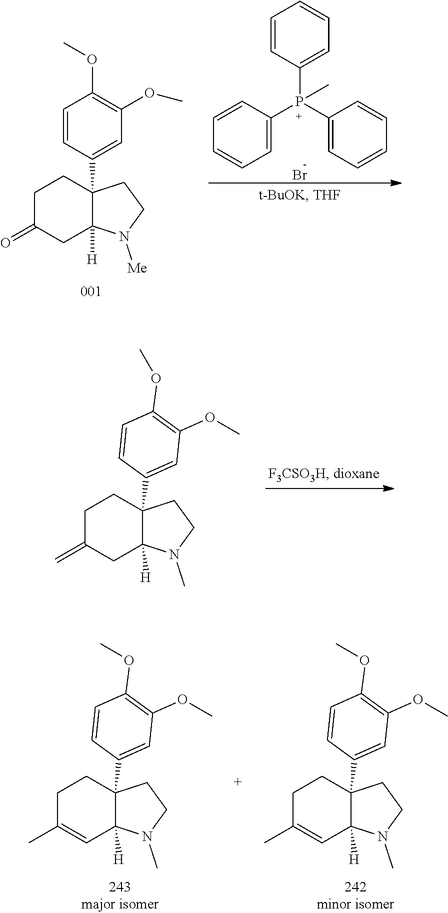

### Step 1: Synthesis of (3aS,7aS)-3a-(3,4-dimethoxyphenyl)-1-methyl-6-methyleneoctahydro-1H-indole

To a solution of methyl(triphenyl)phosphonium bromide (740 mg, 2.07 mmol) in THF (6.0 mL) was added t-BuOK (1 M in THF, 2.07 mL) at 0° C. over 0.5 hours. 001 (300 mg, 1.04 mmol) in THF (1 mL) was added to the mixture. The reaction mixture was allowed to stir at 25° C. for 16 hr and then poured into water (50 mL). The mixture was extracted with ethyl acetate (100 mL). The organic solutions were dried over sodium sulfate, filtered and concentrated. The crude product was purified by reversed-phase HPLC (0.1% FA condition) to give (3aS,7aS)-3a-(3,4-dimethoxyphenyl)-1-methyl-6-methyleneoctahydro-1H-indole (137.7 mg) as a yellow gum. LC-MS (ESI⁺) m/z 288.2 (M+H)⁺. ¹H NMR (400 MHz, CDCl₃) δ 6.88-6.79 (m, 2H), 6.78-6.72 (m, 1H), 4.70 (s, 2H), 3.81 (d, J=6.0 Hz, 6H), 3.37 (ddd, J=5.2, 8.4, 9.8 Hz, 1H), 2.90 (t, J=4.0 Hz, 1H), 2.45 (s, 2H), 2.42-2.35 (m, 4H), 2.16-2.08 (m, 1H), 1.98 (d, J=3.6 Hz, 2H), 1.96-1.86 (m, 3H).

By a Similar Procedure, the Compounds in the Following Table were Prepared:

| Structure | Characterization |
|---|---|
| 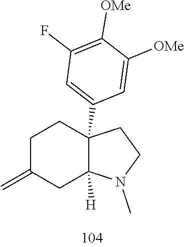 [SMILES: C=C1CC[C@@]2(c3cc(F)c(OC)c(OC)c3)CCN(C)[C@H]2C1] | LC-MS (ESI⁺) m/z 306.1 (M + H)⁺. ¹H NMR (400 MHz, CDCl₃) δ = 6.83-6.69 (m, 2H), 4.84-4.67 (m, 2H), 4.03-3.82 (m, 6H), 3.33-3.20 (m, 1H), 2.73-2.62 (m, 1H), 2.51- 2.26 (m, 6H), 2.24-2.13 (m, 1H), 2.08-1.96 (m, 3H), 1.93-1.75 (m, 2H). |
| 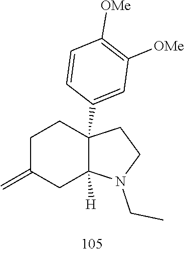 [SMILES: C=C1CC[C@@]2(c3ccc(OC)c(OC)c3)CCN(CC)[C@H]2C1] | LC-MS (ESI⁺) m/z 302.0 (M + H)⁺. ¹H NMR (400 MHz, CDCl₃) δ 6.91- 6.81 (m, 2H), 6.75 (d, J = 8.4 Hz, 1H), 4.64 (d, J = 7.6 Hz, 2H), 3.81 (d, J = 6.0 Hz, 6H), 3.20 (dt, J = 5.2, 8.8 Hz, 1H), 2.98-2.78 (m, 2H), 2.51-2.27 (m, 3H), 2.21 (s, 1H), 2.14-2.06 (m, 1H), 2.04-1.90 (m, 3H), 1.89-1.76 (m, 2H), 1.05 (t, J = 7.2 Hz, 3H). |
| 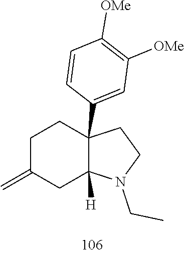 [SMILES: C=C1CC[C@]2(c3ccc(OC)c(OC)c3)CCN(CC)[C@@H]2C1] | LC-MS (ESI⁺) m/z 302.3 (M + H)⁺. ¹H NMR (400 MHz, CDCl₃) δ 6.93- 6.80 (m, 2H), 6.75 (d, J = 8.4 Hz, 1H), 4.63 (d, J = 11.2 Hz, 2H), 3.81 (d, J = 6.0 Hz, 6H), 3.24-3.08 (m, 1H), 2.96-2.75 (m, 2H), 2.41-2.24 (m, 3H), 2.22-2.06 (m, 2H), 2.03- 1.89 (m, 3H), 1.81 (dd, J = 5.6, 10.4 Hz, 2H), 1.03 (t, J = 7.2 Hz, 3H). |
| 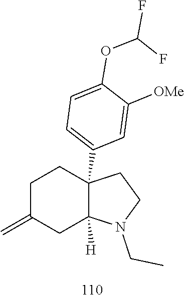 | LC-MS (ESI⁺) m/z 324.1 (M + H)⁺; ¹H NMR (400 MHz, CDCl₃) δ 7.13 (d, J = 8.4 Hz, 1H), 7.06-6.92 (m, 2H), 6.57 (t, J = 75.2 Hz, 1H), 4.76 (d, J = 9.2 Hz, 2H), 3.91 (s, 3H), 3.29 (dt, J = 4.4, 9.2 Hz, 1H), 2.73 (s, 1H), 2.54-2.41 (m, 2H), 2.41- 2.31 (m, 4H), 2.25-2.13 (m, 1H), 2.13-2.03 (m, 2H), 2.02-1.91 (m, 2H), 1.91-1.82 (m, 1H). |
| 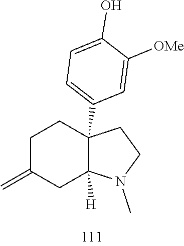 [SMILES: C=C1CC[C@@]2(c3ccc(O)c(OC)c3)CCN(C)[C@H]2C1] | LC-MS (ESI⁺) m/z 274.2 (M + H)⁺. ¹H NMR (400 MHz, CDCl₃) δ 6.85- 6.79 (m, 1H), 6.79-6.73 (m, 2H), 4.75 (d, J = 14.4 Hz, 2H), 3.84-3.84 (m, 3H), 3.65-3.46 (m, 1H), 3.16 (t, J = 4.8 Hz, 1H), 2.61-2.55 (m, 2H), 2.54 (m, 4H), 2.15 (m, 1H), 2.10- 2.00 (m, 3H), 1.99-1.96 (m, 1H), 1.92 (m, 1H). |

### Step 2: Synthesis of (3aS,7aS)-3a-(3,4-dimethoxyphenyl)-1-methyl-6-methyleneoctahydro-1H-indole

To a solution of (3aS,7aS)-3a-(3,4-dimethoxyphenyl)-1-methyl-6-methyleneoctahydro-1H-indole (200 mg, 696 μmol) in dioxane (10 mL) was added TfOH (209 mg, 1.39 mmol). The mixture was allowed to stir at 80° C. for 2 h and then filtered. The filtrate was concentrated under reduced pressure. The residue was purified by prep-HPLC (column: Welch Ultimate C₁₈ 150*25 mm*5 μm; mobile phase: [water (TFA)-ACN]; gradient: 6%-36% B over 13 min) to give a residue. The residue was separated by SFC (“Column: Chiralcel OX-3 50×4.6 mm I.D., 3 μm Mobile phase: Phase A for CO₂, and Phase B for IPA (0.05% DEA); Gradient elution: B in A from 5% to 40% Flow rate: 3 mL/min; Detector: PDA; Column Temp: 35° C.; Back Pressure: 100 Bar”) to give 243 (3aS,7aS)-3a-(3,4-dimethoxyphenyl)-1,6-dimethyl-2,3,3a,4,5,7a-hexahydro-1H-indole (52 mg) as a yellow gum and 242 (3aS,7aS)-3a-(3,4-dimethoxyphenyl)-1,6-dimethyl-2,3,3a,4,7,7a-hexahydro-1H-indole (7 mg) as a yellow gum. LC-MS (ESI⁺) m/z 288.1 (M+H)+; ¹H NMR (400 MHz, CDCl₃) δ 6.85-6.66 (m, 3H), 5.74 (s, 1H), 3.86 (d, J=4.0 Hz, 6H), 3.25-3.12 (m, 1H), 2.62 (d, J=2.4 Hz, 1H), 2.41 (s, 3H), 2.32-2.17 (m, 1H), 2.16-1.99 (m, 3H), 1.81-1.69 (m, 5H), 1.49 (d, J=13.6 Hz, 1H).

### By a Similar Procedure, the Compounds in the Following Table can be Prepared:

| Step 1 | Step 2 |
|---|---|
| 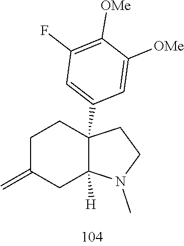 [SMILES: C=C1CC[C@@]2(c3cc(F)c(OC)c(OC)c3)CCN(C)[C@H]2C1] | 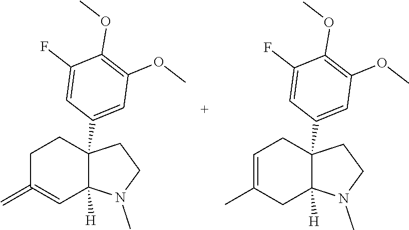 |
| 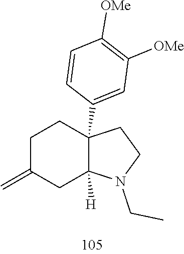 [SMILES: C=C1CC[C@@]2(c3ccc(OC)c(OC)c3)CCN(CC)[C@H]2C1] | 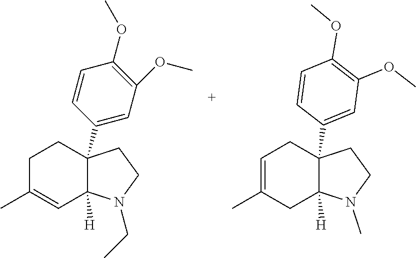 |
| 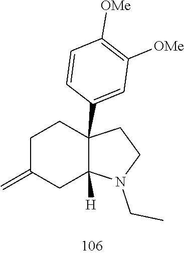 [SMILES: C=C1CC[C@]2(c3ccc(OC)c(OC)c3)CCN(CC)[C@@H]2C1] | 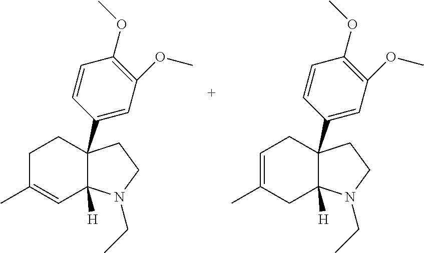 |
| 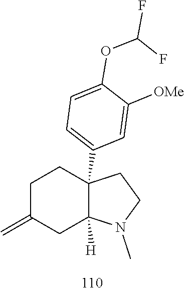 [SMILES: C=C1CC[C@@]2(c3ccc(OC(F)F)c(OC)c3)CCN(C)[C@H]2C1] | 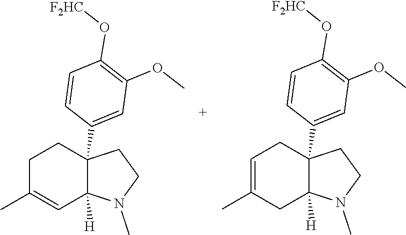 |
| 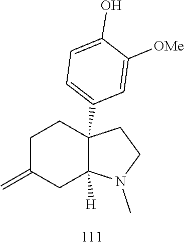 [SMILES: C=C1CC[C@@]2(c3ccc(O)c(OC)c3)CCN(C)[C@H]2C1] | 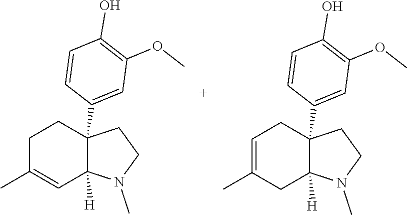 |

### Example 4A-1-methyl-3a-(3,4,5-trimethoxyphenyl)-2,3,7,7a-tetrahydroindol-6-one (144)

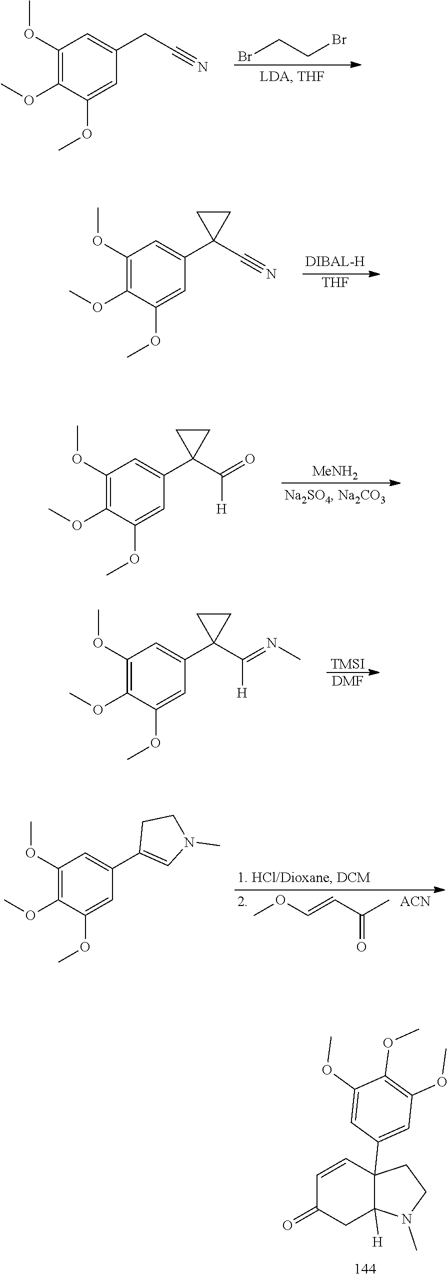

### Step 1 1-(3,4,5-trimethoxyphenyl)cyclopropanecarbonitrile (3)

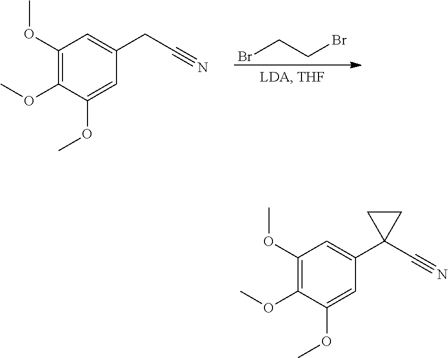

A mixture of 2-(3,4,5-trimethoxyphenyl)acetonitrile (10 g, 48.2 mmol) CAS #13338-63-1,1,2-dibromoethane (10.8 g, 57.9 mmol, 4.37 mL) CAS #106-93-4, LDA (2 M, 60.3 mL) in THF (200 mL) was degassed and purged with N₂ for 3 times, and then the mixture was stirred at −78˜25° C. for 4 hours under N₂ atmosphere. On completion, the mixture was quenched with saturated NH₄Cl (80 mL) and extracted with ethyl acetate (220 mL×3), the combined organic phase was dried over anhydrous sodium sulfate, filtered and concentrated to give a residue. The residue was purified by column chromatography (SiO₂, Petroleum ether/Ethyl acetate=20/1 to 30/1) to give the title compound (4.5 g, 38% yield) as yellow solid.

LC-MS (ESI⁺) m/z 234.1 (M+H)⁺.

¹H NMR (400 MHz, CHLOROFORM-d) δ 6.53 (s, 2H), 3.92-3.80 (m, 9H), 1.73-1.66 (m, 2H), 1.41-1.36 (m, 2H).

### Step 2 1-(3,4,5-trimethoxyphenyl)cyclopropanecarbaldehyde (4)

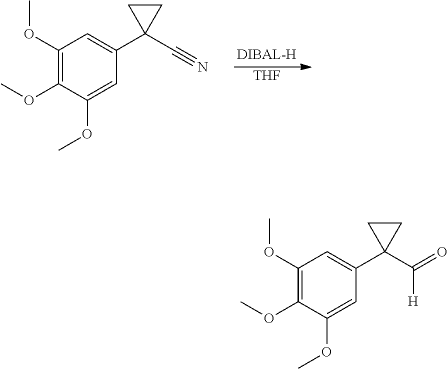 [SMILES: COc1cc(C2(C#N)CC2)cc(OC)c1OC ; COc1cc(C2(C=O)CC2)cc(OC)c1OC ; "THF"]

A mixture of 1-(3,4,5-trimethoxyphenyl)cyclopropanecarbonitrile (4.5 g, 19.3 mmol), DIBAL-H (1 M, 28.9 mL) in THF (50 mL) was degassed and purged with N₂ for 3 times, and then the mixture was stirred at −78˜25° C. for 8 hours under N₂ atmosphere. On completion, the reaction was cautiously quenched by addition of 2 M HCl and organic components were extracted into ethyl acetate (3×65 mL). The combined extracts were washed with water (2×20 mL), brine (2×20 mL) and then dried (Na₂SO₄) filtered and concentrated under reduced pressure to give the residue. The residue was purified by column chromatography (SiO₂, Petroleum ether/Ethyl acetate=5/1 to 5/1) to give the title compound (3.7 g, 79% yield) as yellow solid. LC-MS (ESI⁺) m/z 236.8 (M+H)⁺. ¹H NMR (400 MHz, CHLOROFORM-d) δ 9.28 (s, 1H), 6.53 (s, 2H), 3.94-3.78 (m, 9H), 1.60-1.54 (m, 3H), 1.44-1.37 (m, 2H).

### Step 3 (E)-N-methyl-1-[1-(3,4,5-trimethoxyphenyl)cyclopropyl]methanimine (5)

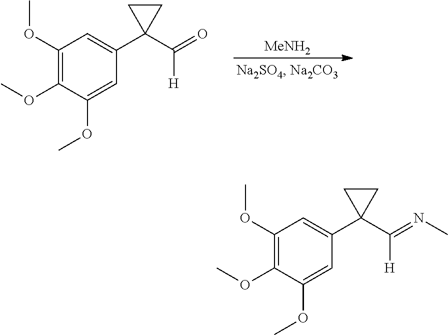

A mixture of 1-(3,4,5-trimethoxyphenyl)cyclopropanecarbaldehyde (3.7 g, 15.6 mmol), Na₂SO₄ (33.4 g, 235 mmol, 23.8 mL), methanamine; hydrochloride (5.29 g, 78.3 mmol) and Na₂CO₃ (6.64 g, 62.6 mmol) in DCM (250 mL) was degassed and purged with N₂ for 3 times, and then the mixture was stirred at 25° C. for 24 hours under N₂ atmosphere. On completion, the reaction liquid was dried with a rotary evaporator after filtering. The crude compound was used into the next step without further purification to give the title compound (2.7 g, 90% yield) as yellow solid.

LC-MS (ESI⁺) m/z 249.9 (M+H)⁺.

¹H NMR (400 MHz, CHLOROFORM-d) δ 7.59 (d, J=1.2 Hz, 1H), 6.56 (s, 2H), 3.97-3.74 (m, 9H), 3.26 (d, J=1.2 Hz, 3H), 1.31-1.26 (m, 2H), 1.19-1.14 (m, 2H).

### Step 4 1-methyl-4-(3,4,5-trimethoxyphenyl)-2,3-dihydropyrrole (6)

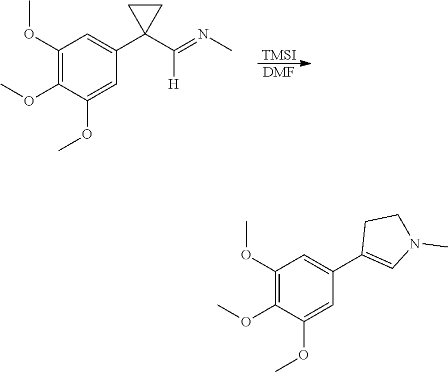

A mixture of (E)-N-methyl-1-[1-(3,4,5-trimethoxyphenyl)cyclopropyl]methanimine (2 g, 8.0 mmol), TMSI (1.61 g, 8.0 mmol, 1.09 mL) in DMF (15 mL) was degassed and purged with N₂ for 3 times, and then the mixture was stirred at 60° C. for 0.5 hours under N₂ atmosphere. On completion, the mixture was quenched with H₂O (18 mL) and extracted with ethyl acetate (20 mL×3), the combined organic phase was dried over anhydrous sodium sulfate, filtered and concentrated to give a residue. Then the aqueous phase was adjusted pH with saturated NaHCO₃ (8 mL) to 8 and extracted with ethyl acetate (20 mL×3), the combined organic phase was dried over anhydrous sodium sulfate, filtered and concentrated to give a residue. The crude compound was used into the next step without further purification to give the title compound (1 g, 30% yield) as yellow oil.

LC-MS (ESI⁺) m/z 250.0 (M+H)⁺.

¹H NMR (400 MHz, CHLOROFORM-d) δ 8.03 (s, 1H), 6.47-6.40 (m, 2H), 6.37 (Br d, J=1.2 Hz, 1H), 3.90-3.80 (m, 9H), 3.17 (Br t, J=9.2 Hz, 2H), 2.67 (s, 2H).

### Step 5 1-methyl-3a-(3,4,5-trimethoxyphenyl)-2,3,7,7a-tetrahydroindol-6-one (144)

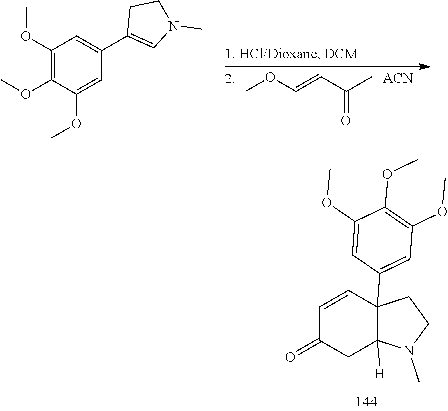

A mixture of 1-methyl-4-(3,4,5-trimethoxyphenyl)-2,3-dihydropyrrole (1 g, 4.0 mmol), (E)-4-methoxybut-3-en-2-one (401 mg, 4.0 mmol, 402.79 uL), HCl/dioxane (4 M, 3 mL) and ACN (6.24 g, 152 mmol, 8 mL) in DCM (9 mL) was degassed and purged with N₂ for 3 times, and then the mixture was stirred at 90° C. for 12 hours under N₂ atmosphere. On completion, the reaction was filtered. The residue was purified by prep-HPLC (basic condition: column: Welch Xtimate C₁₈ 150*25 mm*5 μm; mobile phase: [water (NH₃H₂O)-ACN]; B %: 18%-48%, 8 min) to give the title compound (570 mg, 45% yield) as white solid.

LC-MS (ESI⁺) m/z 317.9 (M+H)⁺. ¹H NMR (400 MHz, CHLOROFORM-d) δ 6.62 (dd, J=2.0, 10.1 Hz, 1H), 6.45 (s, 2H), 6.02 (d, J=10.0 Hz, 1H), 3.84-3.69 (m, 9H), 3.29-3.17 (m, 1H), 2.58 (br s, 1H), 2.50-2.43 (m, 2H), 2.42-2.32 (m, 2H), 2.23 (s, 3H), 2.12 (td, J=8.4, 13.0 Hz, 1H).

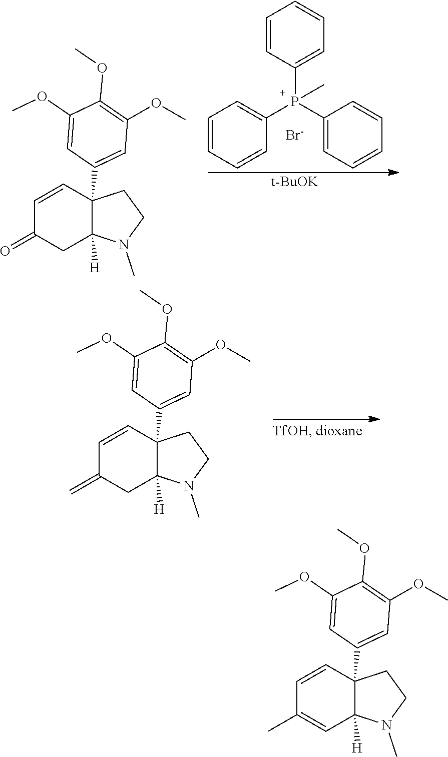

Step 6: Compound 144 is added to a solution of methyl(triphenyl)phosphonium bromide 2.07 mmol) in THF (6.0 mL) is added t-BuOK (1 M in THF, 2.07 mL) at 0° C. over 0.5 hours. 016 (1.04 mmol) in THF (1 mL) is added to the mixture. The reaction mixture was allowed to stir at 25° C. for 16 hr and then poured into water (50 mL). The mixture was extracted with ethyl acetate (100 mL). The organic solutions are dried over sodium sulfate, filtered and concentrated. The crude product is purified by reversed-phase HPLC (0.1% FA condition).

Step 7: The product of Step 6 is added to a solution of 3a-(3,4-dimethoxyphenyl)-1-methyl-6-methylene-2,3,3a,6,7,7a-hexahydro-1H-indole (696 μmol) in dioxane (10 mL) is then added TfOH (1.39 mmol). The mixture was allowed to stir at 80° C. for 2 h and then filtered. The filtrate is concentrated under reduced pressure. The residue is purified by prep-HPLC.

### Example 4B-Compounds 145, 188, and 189

### Step 1 1-methyl-3a-(3,4,5-trimethoxyphenyl)-2,3,4,5,7,7a-hexahydroindol-6-one (145)

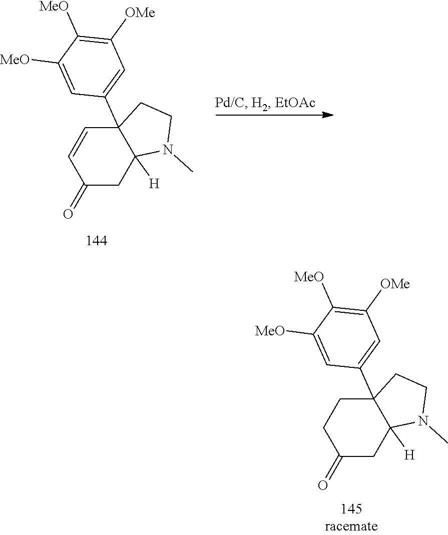 [SMILES: COc1cc(C23C=CC(=O)CC2N(C)CC3)cc(OC)c1OC ; COc1cc(C23CCC(=O)CC2N(C)CC3)cc(OC)c1OC]

A mixture of 1-methyl-3a-(3,4,5-trimethoxyphenyl)-2,3,7,7a-tetrahydroindol-6-one (190 mg, 598 μmol) and Pd/C (10.0 mg, 59.9 μmol, 10% purity) in EtOAc (4 mL) was degassed and purged with H₂ 3 times, and then the mixture was allowed to stir at 25° C. for 12 hours under an atmosphere of H₂. The reaction mixture was filtered. The filtrate was concentrated and purified by prep-HPLC (basic condition: column: Welch Xtimate C₁₈ 150*25 mm*5 μm; mobile phase: [water (ammonia hydroxide v/v)-ACN]; B %: 21%-51%, 2 min) to give 145 (232 mg, 90% yield) as a yellow solid. LC-MS (ESI⁺) m/z 320.2 (M+H)⁺. ¹H NMR (400 MHz, CDCl₃) δ 6.60 (s, 2H), 3.95-3.81 (m, 9H), 3.18-3.11 (m, 1H), 2.95 (t, J=3.2 Hz, 1H), 2.62 (d, J=3.6 Hz, 2H), 2.52-2.41 (m, 1H), 2.38-2.29 (m, 4H), 2.25 (s, 1H), 2.23-2.19 (m, 2H), 2.18-2.10 (m, 2H).

### Step 2 (3aS,7aS)-1-methyl-3a-(3,4,5-trimethoxyphenyl)-2,3,4,5,7,7a-hexahydroindol-6-one (188) and (3aR,7aR)-1-methyl-3a-(3,4,5-trimethoxyphenyl)-2,3,4,5,7,7a-hexahydroindol-6-one (189)

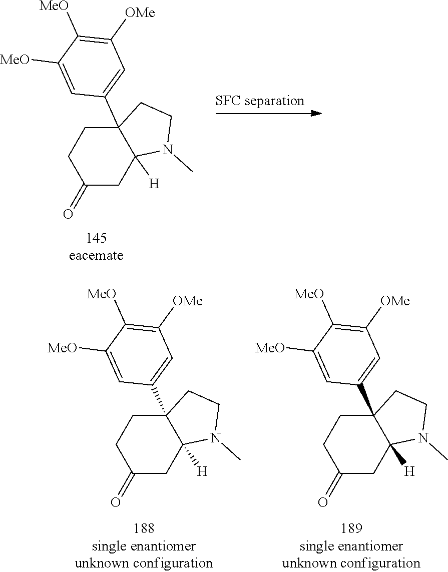 [SMILES: COc1cc(C23CCC(=O)CC2N(C)CC3)cc(OC)c1OC ; COc1cc([C@@]23CCC(=O)C[C@@H]2N(C)CC3)cc(OC)c1OC ; COc1cc([C@]23CCC(=O)C[C@H]2N(C)CC3)cc(OC)c1OC]

Racemic compound 145 was separated by SFC (condition: column: Daicel ChiralPak IG (250*30 mm, 10 μm); mobile phase: [Neu-MeOH]; B %: 25%-25%, C14.55; 146 min) to give 188 (100 mg, 45% yield) as a white solid and 189 (95.0 mg, 43% yield) as a yellow solid.
189: LC-MS (ESI⁺) m/z 320.1 (M+H)⁺. ¹H NMR (400 MHz, CDCl₃) 6.59 (s, 2H), 3.96-3.80 (m, 9H), 3.20-3.10 (m, 1H), 2.95 (Br s, 1H), 2.62 (Br d, J=3.2 Hz, 2H), 2.33 (s, 3H), 2.25 (s, 1H), 2.23-2.19 (m, 2H), 2.18-2.10 (m, 2H), 1.58 (s, 2H).
188: LC-MS (ESI⁺) m/z 320.3 (M+H)⁺. ¹H NMR (400 MHz, CDCl₃) δ 6.59 (s, 2H), 3.96-3.80 (m, 9H), 3.16 (t, J=6.8 Hz, 1H), 2.96 (s, 1H), 2.63 (s, 2H), 2.34 (s, 3H), 2.24 (s, 1H), 2.23-2.19 (m, 2H), 2.18-2.09 (m, 2H), 1.57 (s, 2H).

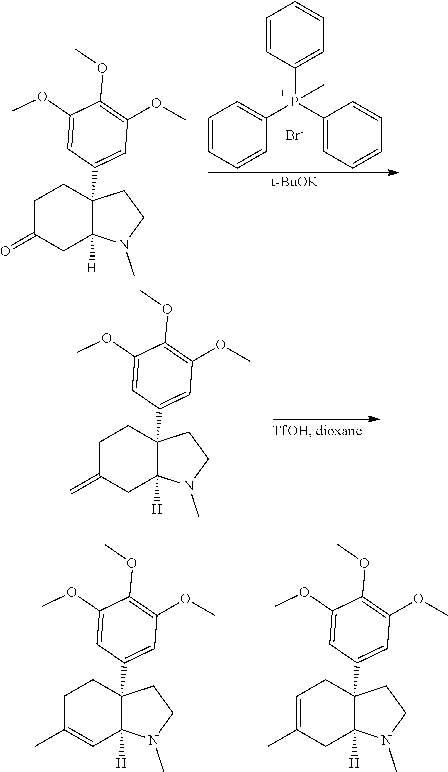

Step 3: Compound 188 and/or Compound 189 is added to a solution of methyl(triphenyl)phosphonium bromide 2.07 mmol) in THF (6.0 mL) is added t-BuOK (1 M in THF, 2.07 mL) at 0° C. over 0.5 hours. 016 (1.04 mmol) in THF (1 mL) is added to the mixture. The reaction mixture was allowed to stir at 25° C. for 16 hr and then poured into water (50 mL). The mixture was extracted with ethyl acetate (100 mL). The organic solutions are dried over sodium sulfate, filtered and concentrated. The crude product is purified by reversed-phase HPLC (0.1% FA condition).

Step 4: The product of Step 3 is added to a solution of 3a-(3,4-dimethoxyphenyl)-1-methyl-6-methylene-2,3,3a,6,7,7a-hexahydro-1H-indole (696 μmol) in dioxane (10 mL) is then added TfOH (1.39 mmol). The mixture was allowed to stir at 80° C. for 2 h and then filtered. The filtrate is concentrated under reduced pressure. The residue is purified by prep-HPLC.

### Example 5A-(3aR,7aS)-3a-(3-fluoro-4,5-dimethoxy-phenyl)-1-methyl-2,3,7,7a-tetrahydroindol-6-one (133)

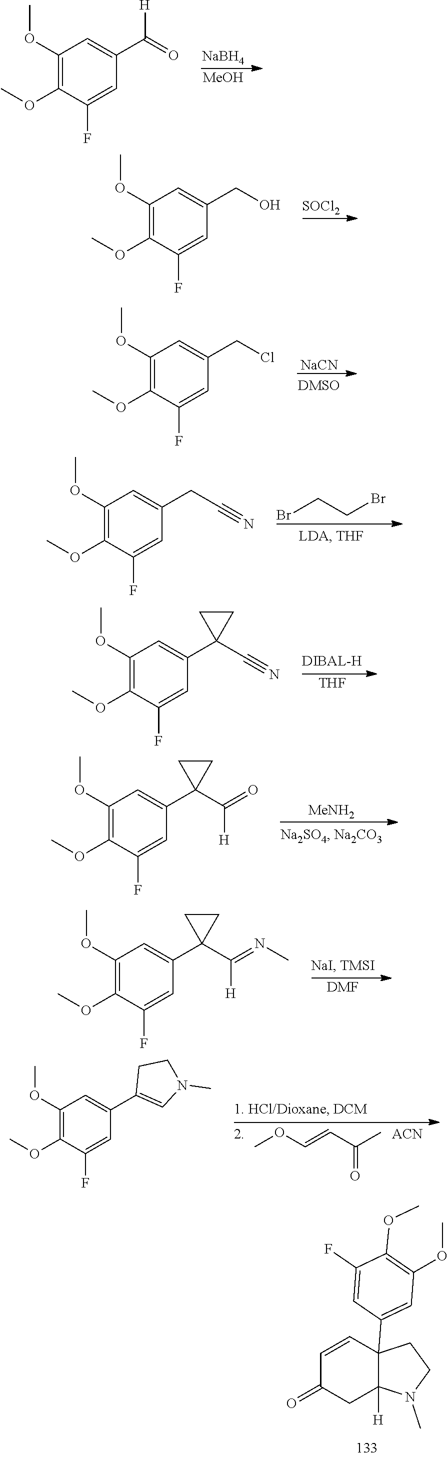

### Step 1-(3-fluoro-4,5-dimethoxy-phenyl)methanol

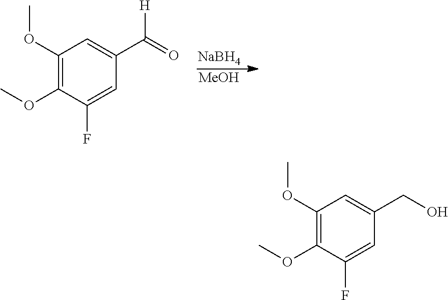

A mixture of 3-fluoro-4,5-dimethoxy-benzaldehyde (4.20 g, 22.81 mmol, CAS: 71924-61-3) in EtOH (60 mL) was added NaBH₄ (949 mg, 25.1 mmol) at 0° C., then the mixture was stirred at 25° C. for 3 hours. On completion, the mixture was poured to the ammonium chloride (10 mL) and extracted with ethyl acetate (30 mL). The organic layer was dried by sodium sulfate, filtered and concentrated in vacuo to give (3-fluoro-4,5-dimethoxy-phenyl)methanol (4.00 g, 89.50% yield, 95% purity) as a yellow oil.

¹H NMR (400 MHz, CDCl₃) δ=6.80-6.56 (m, 2H), 4.55 (s, 2H), 3.90-3.77 (m, 6H).

### Step 2-(chloromethyl)-1-fluoro-2,3-dimethoxy-benzene

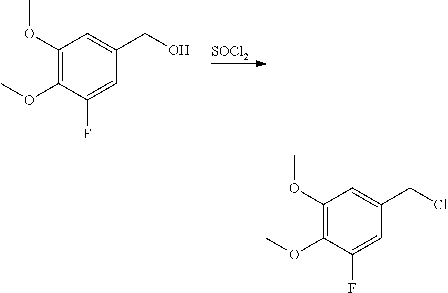

A mixture of (3-fluoro-4,5-dimethoxy-phenyl)methanol (4.00 g, 21.5 mmol) in DCM (40 mL) was added SOCl₂ (6.39 g, 53.7 mmol) at 0° C., then the mixture was stirred at 25° C. for 3 hours. On completion, the mixture was poured to the sodium bicarbonate (10 mL) and extracted with ethyl acetate (100 mL). The organic layer was dried by sodium sulfate, filtered and concentrated in vacuo to give 5-(chloromethyl)-1-fluoro-2,3-dimethoxy-benzene (4.00 g, 91% yield) was obtained as a yellow oil. ¹H NMR (400 MHz, CDCl₃) δ=6.88-6.56 (m, 2H), 4.44 (s, 2H), 3.91-3.80 (m, 6H).

### Step 3-2-(3-fluoro-4,5-dimethoxy-phenyl)acetonitrile

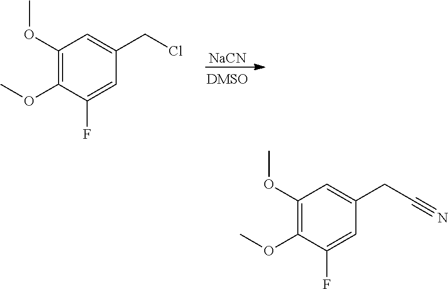

A mixture of 5-(chloromethyl)-1-fluoro-2,3-dimethoxy-benzene (4.00 g, 19.6 mmol) in DMSO (40 mL) was added NaCN (1.92 g, 39.1 mmol), then the mixture was stirred at 25° C. for 16 hours. On completion, the mixture was poured to the saturated sodium bicarbonate solution (40 mL) to adjust pH>7 and extracted with ethyl acetate (100 mL*3), then the organic layer washed by brine (50 mL). The organic layer was dried by sodium sulfate, the residue was purified by column chromatography (SiO₂, Petroleum ether/Ethyl acetate=10/1 to 3/1) to give 2-(3-fluoro-4,5-dimethoxy-phenyl)acetonitrile (3.6 g, 90% yield) as a yellow oil.

¹H NMR (400 MHz, CDCl₃) δ=6.80-6.50 (m, 2H), 3.90-3.75 (m, 6H), 3.62 (s, 2H).

### Step 4-1-(3-bromo-4,5-dimethoxy-phenyl)cyclopropanecarbonitrile

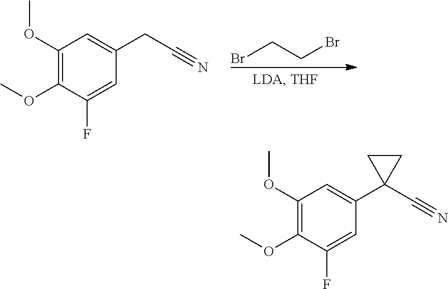

A mixture of 2-(3-fluoro-4,5-dimethoxy-phenyl)acetonitrile (3.60 g, 18.4 mmol) in THF (36 mL) was added LDA (2 M, 23.0 mL), then 1,2-dibromoethane (4.16 g, 22.1 mmol, 1.67 mL) was added above mixture, the mixture was stirred at 25° C. for 2 hours. On completion, the mixture was poured to the water (100 mL) and extracted with ethyl acetate (300 mL). The organic layer was dried by sodium sulfate, filtered and concentrated in vacuo to give the residue. The residue purified by column chromatography (SiO₂, Petroleum ether/Ethyl acetate=10/1 to 3/1) to give 1-(3-fluoro-4,5-dimethoxy-phenyl)cyclopropanecarbonitrile (3.70 g, 14.38 mmol, 78% yield) as a yellow oil. ¹H NMR (400 MHz, CDCl₃) δ=6.67 (t, J=1.8 Hz, 1H), 6.49 (dd, J=2.2, 12.0 Hz, 1H), 3.99-3.74 (m, 6H), 1.72-1.60 (m, 2H), 1.34-1.25 (m, 2H).

### Step 5 1-(3-fluoro-4,5-dimethoxy phenyl)cyclopropanecarbaldehyde

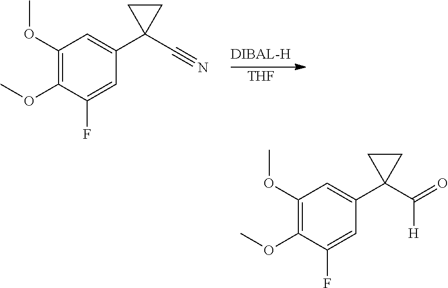 [SMILES: "THF" ; COc1cc(C2(C#N)CC2)cc(F)c1OC ; COc1cc(C2(C=O)CC2)cc(F)c1OC]

A mixture of 1-(3-fluoro-4,5-dimethoxy-phenyl)cyclopropanecarbonitrile (3.70 g, 16.7 mmol), DIBALH (1 M, 25.1 mL) in THF (37 mL) was degassed and purged with N₂ for 3 times, and then the mixture was stirred at 25° C. for 16 hours under N₂ atmosphere. On completion, the mixture was poured to the HCl (2 M, 50 mL) and extracted with ethyl acetate (300 mL). The organic layer was dried by sodium sulfate, filtered and concentrated in vacuo to give 1-(3-fluoro-4,5-dimethoxy-phenyl)cyclopropanecarbaldehyde (3.70 g, 13.2 mmol, 79% yield) as brown oil. ¹H NMR (400 MHz, CDCl₃) δ=9.11 (s, 1H), 7.15-−6.52 (m, 2H), 4.07-3.60 (m, 6H), 1.61-1.40 (m, 2H), 1.37-1.23 (m, 2H).

### Step 6-(E)-1-[1-(3-fluoro-4,5-dimethoxy-phenyl)cyclopropyl]-N-methyl-methanimine

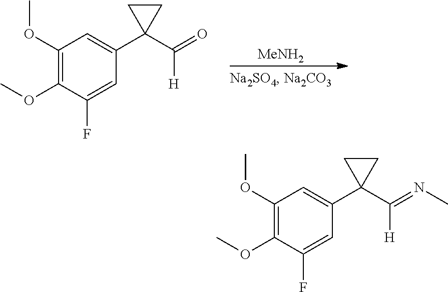

A mixture of 1-(3-fluoro-4,5-dimethoxy-phenyl)cyclopropanecarbaldehyde (3.70 g, 16.5 mmol), methanamine; hydrochloride (5.57 g, 82.5 mmol), Na₂CO₃ (5.25 g, 49.5 mmol) and Na₂SO₄ (35.1 0 g, 247 mmol, 25.1 mL) in DCM (40 mL) was degassed and purged with N₂ for 3 times, and the n the mixture was stirred at 25° C. for 16 hours under N₂ atmosphere. On completion, the reaction mixture was filtered, concentrated in vacuo to give (E)-1-[1-(3-bromo-4,5-dimethoxy-phenyl)cyclopropyl]-N-methyl-methanimine (4.45 g, 86% yield) as a yellow liquid.

¹H NMR (400 MHz, CDCl₃) δ=7.61-7.47 (m, 1H), 6.71 (d, J=1.9 Hz, 2H), 3.91-3.86 (m, 6H), 3.25 (d, J=1.5 Hz, 3H), 1.36-1.22 (m, 2H), 1.20-1.11 (m, 2H).

### Step 7-4-(3-fluoro-4,5-dimethoxy-phenyl)-1-methyl-2,3-dihydropyrrole

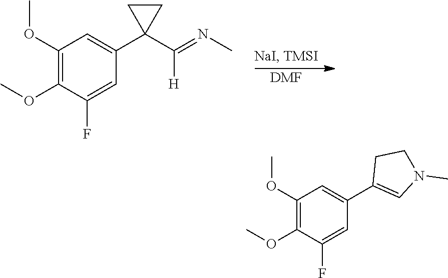

A mixture of (E)-1-[1-(3-fluoro-4,5-dimethoxy-phenyl)cyclopropyl]-N-methyl-methanimine (1.00 g, 4.21 mmol), NaI (63.1 mg, 421. μmol), TMSI (843 mg, 4.21 mmol) in DMF (5 mL) was degassed and purged with N₂ for 3 times, and then the mixture was stirred at 60° C. for 0.5 hr under N₂ atmosphere. On completion, the reaction mixture was filtered, concentrated in vacuo to give 4-(3-fluoro-4,5-dimethoxy-phenyl)-1-methyl-2,3-dihydropyrrole (1.00 g, crude) as a brown oil. LC-MS (ESI⁺) m/z 238.8 (M+H).

Step 8-(3aR,7aS)-3a-(3-fluoro-4,5-dimethoxy-phenyl)-1-methyl-2,3,7,7a-tetrahydroindol-6-one

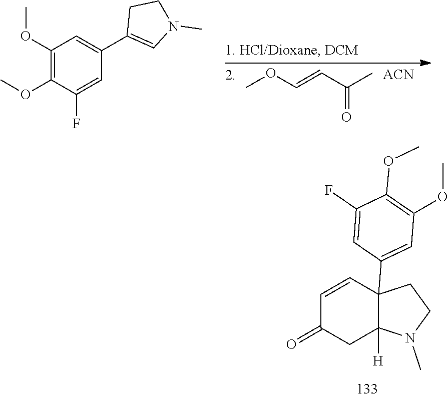

To a solution of 4-(3-fluoro-4,5-dimethoxy-phenyl)-1-methyl-2,3-dihydropyrrole (1.00 g, 1.14 mmol, 27% purity) in DCM (1 mL) was HCl/dioxane (4 M, 10.0 mL) at 25° C. for 10 mins. After addition, the mixture was concentrated in vacuo to give the residue and then (E)-4-methoxybut-3-en-2-one (227 mg, 2.28 mmol, 228 uL) in ACN (10 mL) was added the residue. The mixture was stirred at 80° C. for 16 hours. On completion, the reaction mixture was filtered, concentrated in vacuo to give the residue. The residue was purified by reverse-phase (0.1% FA) to give crude product. The crude product was purified by prep-HPLC (column: Waters xbridge 150*25 mm 10 μm; mobile phase: [water (NH4HCO3)-ACN]; B %: 20%-50%, 9 min) to give (3aR,7aS)-3a-(3-fluoro-4,5-dimethoxy-phenyl)-1-methyl-2,3,7,7a-tetrahydroindol-6-one (9.94 mg, 2.83% yield) was obtained as a yellow solid.

LC-MS (ESI⁺) m/z 306.1 (M+H)⁺.

¹H NMR (400 MHz, CDCl₃) δ=6.67 (dd, J=2.2, 12.8 Hz, 1H), 6.63-6.59 (m, 1H), 6.58 (d, J=1.6 Hz, 1H), 6.05 (d, J=10.4 Hz, 1H), 3.86 (s, 3H), 3.81 (s, 3H), 3.24 (dt, J=2.4, 8.8 Hz, 1H), 2.61-2.56 (m, 1H), 2.56-2.47 (m, 1H), 2.46-2.40 (m, 2H), 2.38-2.30 (m, 1H), 2.25 (s, 3H), 2.19-2.08 (m, 1H).

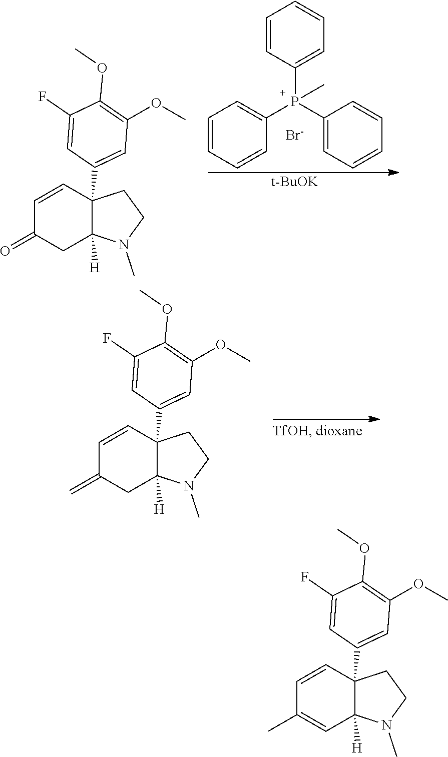

Step 9: Compound 133 is added to a solution of methyl(triphenyl)phosphonium bromide 2.07 mmol) in THF (6.0 mL) is added t-BuOK (1 M in THF, 2.07 mL) at 0° C. over 0.5 hours. 016 (1.04 mmol) in THF (1 mL) is added to the mixture. The reaction mixture was allowed to stir at 25° C. for 16 hr and then poured into water (50 mL). The mixture was extracted with ethyl acetate (100 mL). The organic solutions are dried over sodium sulfate, filtered and concentrated. The crude product is purified by reversed-phase HPLC (0.1% FA condition).

Step 10: The product of Step 9 is added to a solution of 3a-(3,4-dimethoxyphenyl)-1-methyl-6-methylene-2,3,3a,6,7,7a-hexahydro-1H-indole (696 μmol) in dioxane (10 mL) is then added TfOH (1.39 mmol). The mixture was allowed to stir at 80° C. for 2 h and then filtered. The filtrate is concentrated under reduced pressure. The residue is purified by prep-HPLC.

### Example 5B-Compounds 134, 194, 195

### Step 1-3a-(3-fluoro-4,5-dimethoxy-phenyl)-1-methyl-2,3,4,5,7,7a-hexahydroindol-6-one (134)

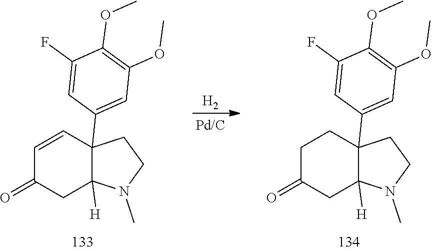 [SMILES: COc1cc(C23C=CC(=O)CC2N(C)CC3)cc(F)c1OC ; "H2" ; COc1cc(C23CCC(=O)CC2N(C)CC3)cc(F)c1OC]

A mixture of 133 (70.00 mg, 229 μmol), Pd/C (10 mg, 10% purity) in ethyl acetate (2 mL) was degassed and purged with N₂ 3 times, and then the mixture was allowed to stir at 25° C. for 1 hr under an atmosphere of H₂. The reaction mixture was filtered, concentrated in vacuo, and purified by prep-HPLC (column: Welch Xtimate C₁₈ 150*25 mm*5 μm; mobile phase: [water (NH₃H₂O)-ACN]; B %: 30%-60%, 8 min) to give 134 (20 mg, 61.8 μmol, 26% yield, 95% purity) as a white solid. LC-MS (ESI⁺) m/z 308.2 (M+H)^{+ 1}H NMR (400 MHz, CDCl₃) δ=6.83-6.67 (m, 1H), 6.62 (s, 1H) 4.07-3.69 (m, 6H), 3.15-2.95 (m, 1H), 2.91-2.80 (m, 1H), 2.52 (d, J=2.8 Hz, 2H), 2.45-2.33 (m, 1H), 2.31-2.21 (m, 4H), 2.18-1.93 (m, 5H).

### Step 2-(3aS,7aS)-3a-(3-fluoro-4,5-dimethoxy-phenyl)-1,7a-dimethyl-3,4,5,7-tetrahydro-2H-indol-6-one (194) & (3aR,7aR)-3a-(3-fluoro-4,5-dimethoxy-phenyl)-1,7a-dimethyl-3,4,5,7-tetrahydro-2H-indol-6-one (195)

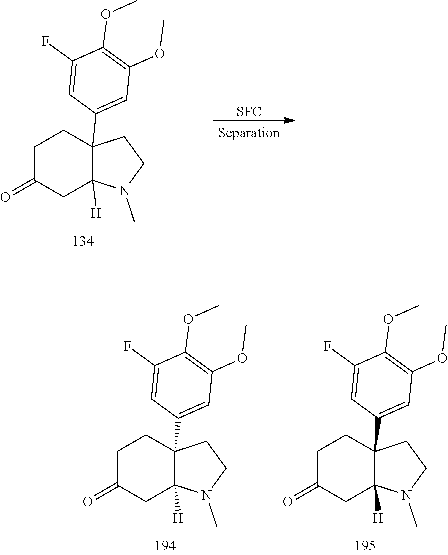 [SMILES: COc1cc(C23CCC(=O)CC2N(C)CC3)cc(F)c1OC ; "SFC" ; COc1cc([C@@]23CCC(=O)C[C@@H]2N(C)CC3)cc(F)c1OC ; COc1cc([C@]23CCC(=O)C[C@H]2N(C)CC3)cc(F)c1OC]

Racemic 134 was purified by SFC (column: Daicel ChiralPak IG (250*30 mm, 10 μm); mobile phase: [0.1% NH₃H₂O MEOH]; B %: 20%-20%, C12.05; 109 min) to give 194 (4.09 mg, 18% yield, 97% purity) as a white solid and 195 (4.92 mg, 22% yield, 94% purity) as a white solid.
194: LC-MS (ESI⁺) m/z 308.2 (M+H)^{+ 1}H NMR (400 MHz, CDCl₃) δ=6.77 (br d, J=12.8 Hz, 1H), 6.70 (s, 1H), 3.92 (br d, J=13.6 Hz, 6H), 3.14 (t, J=6.4 Hz, 1H), 2.90 (s, 1H), 2.70-2.53 (m, 2H), 2.52-2.40 (m, 1H), 2.38-2.27 (m, 4H), 2.26-2.13 (m, 3H), 2.13-2.06 (m, 2H)
195: LC-MS (ESI⁺) m/z 308.2 (M+H); ¹H NMR (400 MHz, CDCl₃) δ=6.77 (br d, J=12.8 Hz, 1H), 6.70 (s, 1H), 3.92 (br d, J=13.6 Hz, 6H), 3.14 (t, J=6.4 Hz, 1H), 2.90 (s, 1H), 2.70-2.53 (m, 2H), 2.52-2.40 (m, 1H), 2.38-2.27 (m, 4H), 2.26-2.13 (m, 3H), 2.13-2.06 (m, 2H).

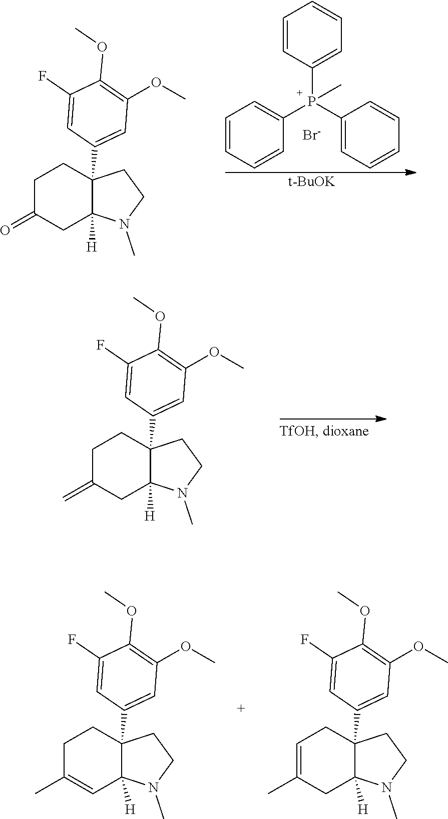

Step 3: Compound 194 is added to a solution of methyl(triphenyl)phosphonium bromide 2.07 mmol) in THF (6.0 mL) is added t-BuOK (1 M in THF, 2.07 mL) at 0° C. over 0.5 hours. 016 (1.04 mmol) in THF (1 mL) is added to the mixture. The reaction mixture was allowed to stir at 25° C. for 16 hr and then poured into water (50 mL). The mixture was extracted with ethyl acetate (100 mL). The organic solutions are dried over sodium sulfate, filtered and concentrated. The crude product is purified by reversed-phase HPLC (0.1% FA condition).

Step 4: The product of Step 3 is added to a solution of 3a-(3,4-dimethoxyphenyl)-1-methyl-6-methylene-2,3,3a,6,7,7a-hexahydro-1H-indole (696 μmol) in dioxane (10 mL) is then added TfOH (1.39 mmol). The mixture was allowed to stir at 80° C. for 2 h and then filtered. The filtrate is concentrated under reduced pressure. The residue is purified by prep-HPLC.

### Example 6A: Synthesis of 149 and 150

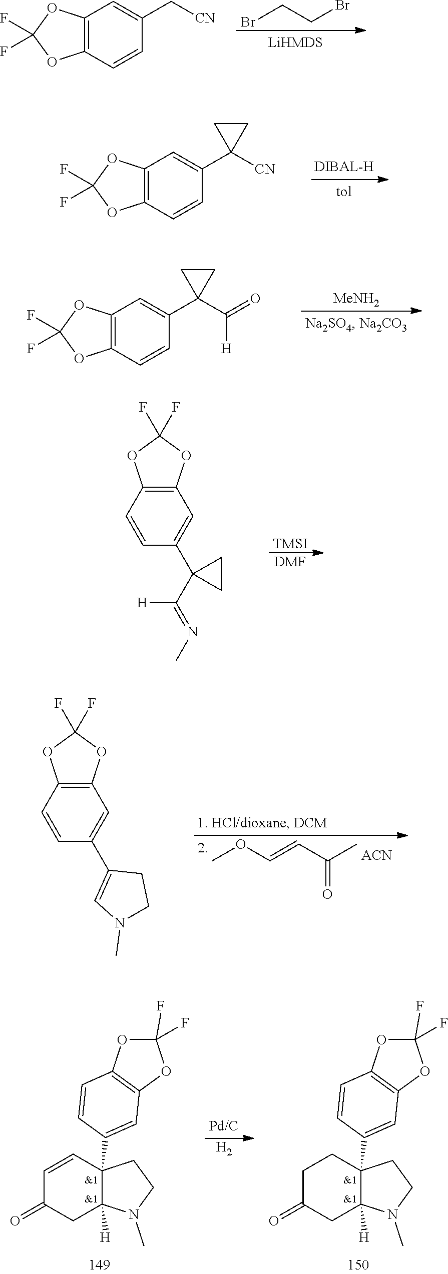

### Step 1: Synthesis of 1-(2,2-difluorobenzo[d][1,3]dioxol-5-yl)cyclopropane-1-carbonitrile

To a solution of 1,2-dibromoethane (19.0 g, 101 mmol, 7.7 mL) in THF (300 mL) was added LiHMDS (1 M, 177.5 mL). The mixture was allowed to stir at −20° C. for 30 min, then 2-(2,2-difluoro-1,3-benzodioxol-5-yl)acetonitrile (10 g, 50.7 mmol) was added. The reaction mixture was allowed to stir and warm from −20° C. to 25° C. over 2 hr. The reaction mixture was quenched by pouring into cold saturated aqueous NH₄Cl solution (300 mL). The aqueous solution was extracted with ethyl acetate (300 mL×2). The organic solutions were combined, dried over anhydrous Na₂SO₄, filtered and concentrated under reduced pressure. The residue was purified by column chromatography (SiO₂, Petroleum ether/Ethyl acetate=10/1 to 10/1) to give 1-(2,2-difluorobenzo[d][1,3]dioxol-5-yl)cyclopropane-1-carbonitrile (7.5 g, 50%) as yellow oil. ¹H NMR (400 MHz, CDCl₃) δ 7.09-6.91 (m, 3H), 1.70-1.65 (m, 2H), 1.35-1.26 (m, 2H).

### Step 2: Synthesis of 1-(2,2-difluorobenzo[d][1,3]dioxol-5-yl)cyclopropane-1-carbaldehyde

To a solution of 1-(2,2-difluorobenzo[d][1,3]dioxol-5-yl)cyclopropane-1-carbonitrile (7.5 g, 33.6 mmol) in toluene (70 mL) was added DIBAL-H (1 M, 40.3 mL). The mixture was allowed to stir at 0-25° C. for 8 hr. The reaction mixture was quenched by pouring into cold saturated aqueous 2N HCl (50 mL). The aqueous solution was extracted with ethyl acetate (50 mL×2). The organic solutions were combined, dried over anhydrous Na₂SO₄, filtered and concentrated under reduced pressure. The residue was purified by column chromatography (SiO₂, Petroleum ether/Ethyl acetate=10/1 to 10/1) to give 1-(2,2-difluorobenzo[d][1,3]dioxol-5-yl)cyclopropane-1-carbaldehyde (6.2 g, 85%) as yellow oil. ¹H NMR (400 MHz, CDCl₃) δ 9.33 (s, 1H), 7.64-7.38 (m, 1H), 7.35-7.27 (m, 1H), 7.24 (d, J=1.6 Hz, 1H), 1.88-1.81 (m, 2H), 1.69-1.62 (m, 2H).

### Step 3: Synthesis of (E)-1-(1-(2,2-difluorobenzo[d][1,3]dioxol-5-yl)cyclopropyl)-N-methylmethanamine

To a solution of 1-(2,2-difluorobenzo[d][1,3]dioxol-5-yl)cyclopropane-1-carbaldehyde (6.2 g, 27.4 mmol) in DCM (150 mL) was added methanamine (2 M, 137 mL) and Na₂SO₄ (17.5 g, 123 mmol, 12.5 mL). The mixture was allowed to stir at 25° C. for 10 hr. The reaction mixture was quenched by adding it to a cold saturated aqueous H₂O solution (150 mL). The aqueous solution was extracted with ethyl acetate (150 mL×2). The organic solutions were combined, dried over anhydrous Na₂SO₄, filtered and concentrated under reduced pressure to give (E)-1-(1-(2,2-difluorobenzo[d][1,3]dioxol-5-yl)cyclopropyl)-N-methylmethanamine (6.7 g) as yellow oil. ¹H NMR (400 MHz, CDCl₃) δ 9.02 (s, 1H), 6.99-6.86 (m, 3H), 3.16 (d, J=1.2 Hz, 3H), 1.23-1.16 (m, 2H), 1.12-1.05 (m, 2H).

### Step 4: Synthesis of 4-(2,2-difluorobenzo[d][1,3]dioxol-5-yl)-1-methyl-2,3-dihydro-1H-pyrrole

A mixture of (E)-1-(1-(2,2-difluorobenzo[d][1,3]dioxol-5-yl)cyclopropyl)-N-methylmethanamine (500 mg, 2.09 mmol) and TMSI (418 mg, 2.09 mmol, 284 uL) in DMF (4 mL) was degassed and purged with N₂ 3 times, and then the reaction mixture was allowed to stir at 60° C. for 1 hr under an atmosphere of N₂. The reaction mixture was quenched by adding it to water (10 mL). The aqueous solution was extracted with ethyl acetate (10 mL×2). The organic solutions were combined, dried over anhydrous Na₂SO₄, filtered and concentrated under reduced pressure to give 4-(2,2-difluorobenzo[d][1,3]dioxol-5-yl)-1-methyl-2,3-dihydro-1H-pyrrole (1.5 g, 60%) as a yellow oil. ¹H NMR (400 MHz, CDCl₃) δ 6.95-6.89 (m, 2H), 6.85-6.80 (m, 1H), 6.36 (s, 1H), 2.68 (s, 3H).

### Step 5: Synthesis of 149

A mixture of 4-(2,2-difluorobenzo[d][1,3]dioxol-5-yl)-1-methyl-2,3-dihydro-1H-pyrrole (1.2 g, 5.02 mmol), (E)-4-methoxybut-3-en-2-one (753 mg, 7.52 mmol, 755 uL), HCl/dioxane (4 M, 123 mL), DCM (5 mL) and ACN (5 mL) was degassed and purged with N₂ 3 times, and then the reaction mixture was allowed to stir at 90° C. for 8 hr under an atmosphere of N₂. The reaction mixture was filtered and the filtrate was concentrated under reduced pressure. The residue was purified by prep-HPLC (column: Welch Xtimate C₁₈ 150*25 mm*5 μm; mobile phase: [water (HCl)-ACN]; B %: 7%-37%, 8 min) to give 149 (1 g, 46%) as a white solid. LC-MS (ESI⁺) m/z 308.0 (M+H)⁺. ¹H NMR (400 MHz, DMSO-d₆) δ 10.69-10.47 (m, 1H), 7.72 (s, 1H), 7.45 (d, J=8.4 Hz, 1H), 7.28 (dd, J=1.6, 8.5 Hz, 1H), 7.04 (d, J=9.2 Hz, 1H), 6.27 (d, J=10.4 Hz, 1H), 4.06 (s, 1H), 3.57 (dd, J=7.6, 11.2 Hz, 1H), 3.46-3.39 (m, 1H), 2.96 (d, J=4.4 Hz, 3H), 2.84-2.71 (m, 2H), 2.49-2.41 (m, 2H).

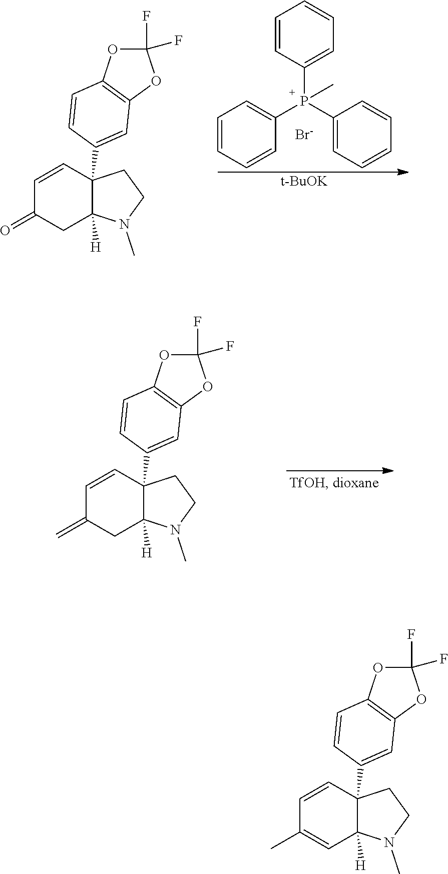

Step 5B: Compound 149 is added to a solution of methyl(triphenyl)phosphonium bromide 2.07 mmol) in THF (6.0 mL) is added t-BuOK (1 M in THF, 2.07 mL) at 0° C. over 0.5 hours. 016 (1.04 mmol) in THF (1 mL) is added to the mixture. The reaction mixture was allowed to stir at 25° C. for 16 hr and then poured into water (50 mL). The mixture was extracted with ethyl acetate (100 mL). The organic solutions are dried over sodium sulfate, filtered and concentrated. The crude product is purified by reversed-phase HPLC (0.1% FA condition).

Step 5C: The product of Step 5B is added to a solution of 3a-(3,4-dimethoxyphenyl)-1-methyl-6-methylene-2,3,3a,6,7,7a-hexahydro-1H-indole (696 mol) in dioxane (10 mL) is then added TfOH (1.39 mmol). The mixture was allowed to stir at 80° C. for 2 h and then filtered. The filtrate is concentrated under reduced pressure. The residue is purified by prep-HPLC.

### Step 6: Synthesis of 150

To a solution of 149 (140 mg, 455 mol) in EtOAc (3 mL) was added Pd/C (15 mg, 455 μmol, 10% purity). The reaction mixture was allowed to stir at 25° C. for 2 hr under an atmosphere of H₂. The reaction mixture was filtered and the filtrate was concentrated under reduced. The residue was purified by prep-HPLC (column: Welch Xtimate C₁₈ 150*25 mm*5 μm; mobile phase: [water (ammonia hydroxide v/v)-ACN]; B %: 39%-69%, 2 min) to give 150 (80 mg, 54%) as white oil. LC-MS (ESI⁺) m/z 303.1 (M+H)⁺. ¹H NMR (400 MHz, CDCl₃) δ 7.15-7.08 (m, 2H), 7.06-7.02 (m, 1H), 3.19 (d, J=2.4 Hz, 1H), 2.96 (s, 1H), 2.61 (d, J=2.4 Hz, 2H), 2.52-2.42 (m, 1H), 2.35 (s, 3H), 2.30-2.22 (m, 1H), 2.20-2.09 (m, 4H), 1.71-1.41 (m, 1H).

### Example 6B: Prep-HPLC Separation of 150 to Give 232 and 233

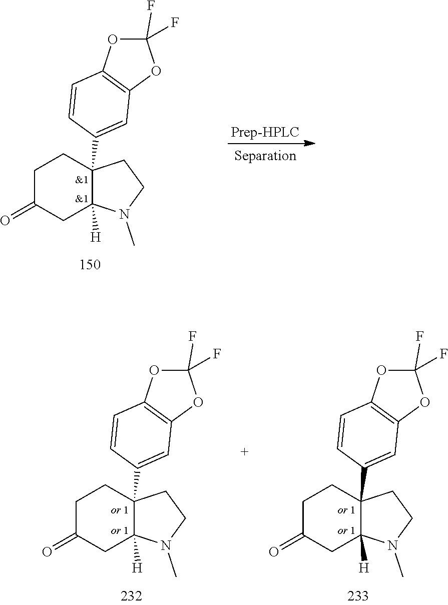 [SMILES: CN1CC[C@]2(c3ccc4c(c3)OC(F)(F)O4)CCC(=O)C[C@H]12 ; "Prep-HPLC" ; CN1CC[C@]2(c3ccc4c(c3)OC(F)(F)O4)CCC(=O)C[C@H]12 ; CN1CC[C@@]2(c3ccc4c(c3)OC(F)(F)O4)CCC(=O)C[C@@H]12]

The enantiomers of 150 were separated by prep-HPLC (column: DAICEL CHIRALPAK AD (250 mm*30 mm, 10 μm); mobile phase: [Neu-MeOH]; B %: 25%-25%, C6.0; 54 min) to 232 (34 mg, 42%) as yellow oil and 233 (35 mg, 53%) as white oil.
232: LC-MS (ESI⁺) m/z 310.1 (M+H)⁺. ¹H NMR (400 MHz, CDCl₃) δ 7.18-7.08 (m, 2H), 7.07-7.02 (m, 1H), 3.23-3.11 (m, 1H), 2.93 (s, 1H), 2.67-2.54 (m, 2H), 2.53-2.42 (m, 1H), 2.40-2.29 (m, 4H), 2.26-2.22 (m, 1H), 2.21-2.16 (m, 1H), 2.16-2.07 (m, 3H).
233: LC-MS (ESI⁺) m/z 310.1 (M+H)⁺. ¹H NMR (400 MHz, CDCl₃) δ 7.15-7.08 (m, 2H), 7.06-7.02 (m, 1H), 3.21-3.11 (m, 1H), 2.93 (t, J=3.2 Hz, 1H), 2.60 (dd, J=3.6, 6.1 Hz, 2H), 2.52-2.43 (m, 1H), 2.39-2.29 (m, 4H), 2.28-2.22 (m, 1H), 2.21-2.16 (m, 1H), 2.16-2.06 (m, 3H).

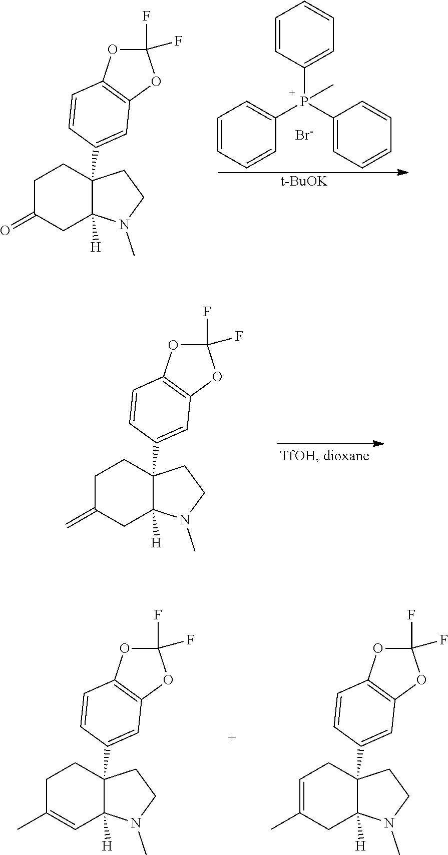

Step 7: Compound 232 is added to a solution of methyl(triphenyl)phosphonium bromide 2.07 mmol) in THF (6.0 mL) is added t-BuOK (1 M in THF, 2.07 mL) at 0° C. over 0.5 hours. 016 (1.04 mmol) in THF (1 mL) is added to the mixture. The reaction mixture was allowed to stir at 25° C. for 16 hr and then poured into water (50 mL). The mixture was extracted with ethyl acetate (100 mL). The organic solutions are dried over sodium sulfate, filtered and concentrated. The crude product is purified by reversed-phase HPLC (0.1% FA condition).

Step 8: The product of Step 7 is added to a solution of 3a-(3,4-dimethoxyphenyl)-1-methyl-6-methylene-2,3,3a,6,7,7a-hexahydro-1H-indole (696 μmol) in dioxane (10 mL) is then added TfOH (1.39 mmol). The mixture was allowed to stir at 80° C. for 2 h and then filtered. The filtrate is concentrated under reduced pressure. The residue is purified by prep-HPLC.

### Example 7: Synthesis of 340, 341, 342

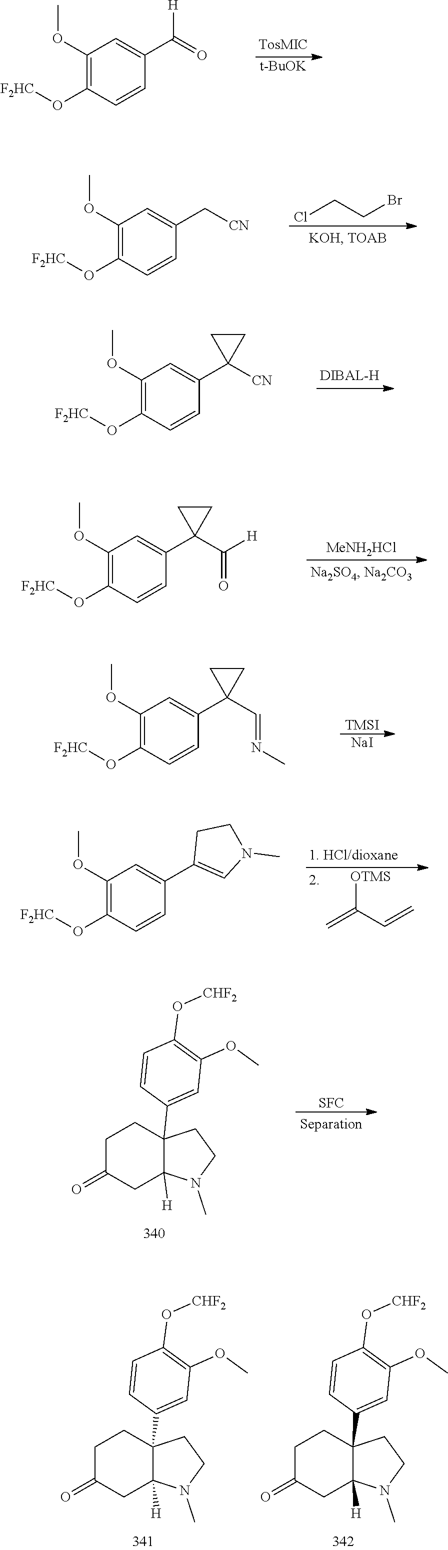

### Step 1-2-[4-(difluoromethoxy)-3-methoxy-phenyl]acetonitrile

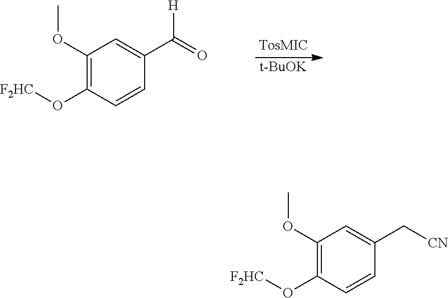

To a solution of t-BuOK (1 M in THF, 395 mL) was added 1-(isocyanomethylsulfonyl)-4-methyl-benzene (38.6 g, 197 mmol) in THF (600 mL) at −70° C. The mixture was allowed to stir for 1 hour, treated with a solution of 4-(difluoromethoxy)-3-methoxy-benzaldehyde (25 g, 123 mmol) in THF (100 mL) dropwise, and was allowed to continue to stir for 2 hours at 60° C. the mixture was cooled and then MeOH (500 mL was added. Then the reaction mixture was allowed to stir at 60° C. for 2 hours. The reaction mixture was concentrated in vacuo. The mixture was poured to the water (500 mL) and extracted with ethyl acetate (1500 mL). The organic solutions were combined, dried over sodium sulfate, filtered and concentrated in vacuo. The residue was purified by column chromatography (SiO₂, petroleum ether:ethyl acetate=5:1) to give 2-[4-(difluoromethoxy)-3-methoxy-phenyl]acetonitrile (21 g, 38% yield) as a yellow oil. ¹H NMR (400 MHz, CDCl₃) δ 7.09 (d, J=8.0 Hz, 1H), 6.86 (d, J=2.0 Hz, 1H), 6.81 (dd, J=2.0, 8.2 Hz, 1H), 6.47 (t, J=74.8 Hz, 1H), 3.83 (s, 3H), 3.67 (s, 2H).

### Step 2-1-[4-(difluoromethoxy)-3-methoxy-phenyl]cyclopropanecarbonitrile

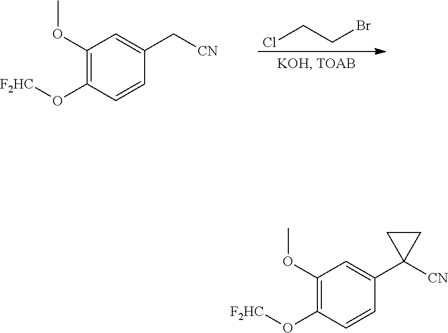

A mixture of 2-[4-(difluoromethoxy)-3-methoxy-phenyl]acetonitrile (8.0 g, 37.5 mmol), 1-bromo-2-chloro-ethane (5.38 g, 37.5 mmol, 3.11 mL) and tetraoctylammonium bromide (4.10 g, 7.5 mmol) in KOH (17.8 M, 10.5 mL) was degassed and purged with N₂ 3 times, and then the mixture was allowed to stir at 70° C. for 16 hours under an atmosphere of N₂. The mixture was poured into water (200 mL) and extracted with ethyl acetate (500 mL). The organic solutions were combined, dried over sodium sulfate, filtered and concentrated in vacuo. The residue was purified by column chromatography (SiO₂, petroleum ether:ethyl acetate=10:1) to give 1-[4-(difluoromethoxy)-3-methoxy-phenyl]cyclopropanecarbonitrile (12.7 g, 67% yield) as an off-white solid. ¹H NMR (400 MHz, CDCl₃) δ 7.17-7.11 (m, 1H), 7.02 (d, J=2.0 Hz, 1H), 6.78 (dd, J=2.0, 8.4 Hz, 1H), 6.55 (t, J=75.2 Hz, 1H), 3.93 (s, 3H), 1.80-1.71 (m, 2H), 1.46-1.36 (m, 2H).

### Step 3-1-[4-(difluoromethoxy)-3-methoxy-phenyl]cyclopropanecarbaldehyde

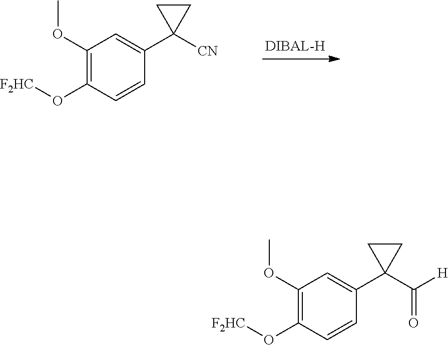 [SMILES: COc1cc(C2(C#N)CC2)ccc1OC(F)F ; COc1cc(C2(C=O)CC2)ccc1OC(F)F]

To a solution of 1-[4-(difluoromethoxy)-3-methoxy-phenyl]cyclopropanecarbonitrile (20 g, 83.6 mmol) in THF (200 mL) was added DIBAL-H (1 M, 125 mL) over 30 minutes at 0° C. under and atmosphere of N₂. The mixture was allowed to stir at 25° C. for 16 hours under N₂ atmosphere. The reaction mixture was poured into dilute hydrochloric acid solution (2 M, 300 mL) and extracted with ethyl acetate (600 mL). The organic solutions were combined and concentrated in vacuo to give 1-[4-(difluoromethoxy)-3-methoxy-phenyl]cyclopropanecarbaldehyde (20 g, 93% yield) as a yellow solid. ¹H NMR (400 MHz, CDCl₃) δ 9.11 (s, 1H), 7.07 (d, J=8.0 Hz, 1H), 6.85 (s, 1H), 6.79 (d, J=8.0 Hz, 1H), 6.47 (t, J=75.2 Hz, 1H), 3.82 (s, 3H), 1.54-1.49 (m, 2H), 1.38-1.30 (m, 2H).

### Step 4-(E)-1-[1-[4-(difluoromethoxy)-3-methoxy-phenyl]cyclopropyl]-N-methyl-methanimine

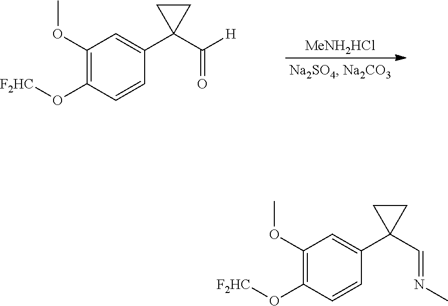 [SMILES: "MeNH2HCl" ; COc1cc(C2(C=O)CC2)ccc1OC(F)F ; C/N=C/C1(c2ccc(OC(F)F)c(OC)c2)CC1]

A mixture of 1-[4-(difluoromethoxy)-3-methoxy-phenyl]cyclopropanecarbaldehyde (20 g, 82.5 mmol), Na₂CO₃ (26.2 g, 247 mmol), Na₂SO₄ (117 g, 825 mmol) and methanamine hydrochloride (27.8 g, 412 mmol) in DCM (200 mL) was degassed and purged with N₂ 3 times, and then the mixture was allowed to stir at 25° C. for 16 hours under an atmosphere of N₂. The reaction mixture was filtered, and the filtrate was concentrated in vacuo to give (E)-1-[1-[4-(difluoromethoxy)-3-methoxy-phenyl]cyclopropyl]-N-methyl-methanimine (20 g, 90% yield) as a yellow oil. ¹H NMR (400 MHz, CDCl₃) δ 7.41 (d, J=1.6 Hz, 1H), 7.02 (d, J=8.0 Hz, 1H), 6.85 (d, J=2.0 Hz, 1H), 6.81 (dd, J=2.0, 8.0 Hz, 1H), 6.45 (t, J=75.2 Hz, 1H), 3.80 (s, 3H), 3.17 (d, J=1.6 Hz, 3H), 1.23-1.16 (m, 2H), 1.12-1.06 (m, 2H).

### Step 5-4-[4-(difluoromethoxy)-3-methoxy-phenyl]-1-methyl-2,3-dihydropyrrole

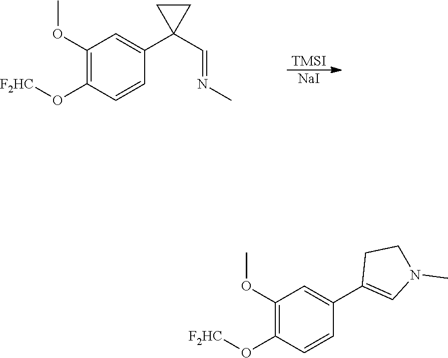 [SMILES: "TMSI" ; C/N=C/C1(c2ccc(OC(F)F)c(OC)c2)CC1 ; "NaI" ; COc1cc(C2=CN(C)CC2)ccc1OC(F)F]

To a solution of (E)-1-[1-[4-(difluoromethoxy)-3-methoxy-phenyl]cyclopropyl]-N-methyl-methanimine (2 g, 7.84 mmol) in DMF (20 mL) was added TMSI (1.57 g, 7.84 mmol, 1.07 mL) and NaI (117 mg, 783 μmol). The reaction mixture was allowed to stir at 60° C. for 30 minutes. The mixture was poured into water (250 mL) and extracted with ethyl acetate (600 mL). To the aqueous phase was added saturated sodium bicarbonate solution to adjust pH>7, and the solution was extracted with ethyl acetate (300 mL). The organic solutions were combined, dried over sodium sulfate, filtered and concentrated in vacuo to give 4-[4-(difluoromethoxy)-3-methoxy-phenyl]-1-methyl-2,3-dihydropyrrole (7 g, 33% yield) as a yellow oil. LC-MS (ESI⁺) m/z 256.1 (M+H)+

### Step 6-3a-[4-(difluoromethoxy)-3-methoxy-phenyl]-1-methyl-2,3,4,5,7,7a-hexahydroindol-6-one (340)

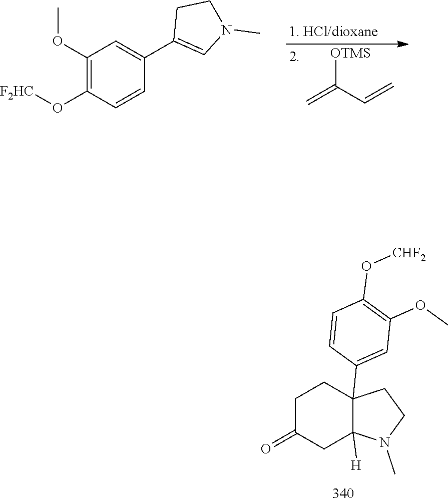

To a solution of 4-[4-(difluoromethoxy)-3-methoxy-phenyl]-1-methyl-2,3-dihydropyrrole (7 g, 15.6 mmol) in DCM (60 mL) was added HCl/dioxane (4 M, 13.6 mL). The reaction mixture was allowed to stir at 20° C. for 30 minutes. The reaction mixture was concentrated in vacuo. The residue was redissolved in ACN (60 mL) and to this solution was added trimethyl(1-methylene allyloxy) silane (3.34 g, 23.4 mmol, 4.07 mL). The mixture was allowed to stir at 80° C. for 16 hours. The mixture was poured into 4 M NaOH solution (100 mL) and extracted with ethyl acetate (250 mL). The organic solutions were combined, dried over sodium sulfate, filtered and concentrated in vacuo. The residue was purified by column chromatography (SiO₂, petroleum ether:ethyl acetate=1:1) to give 340 (4 g, 76% yield) as a yellow oil. LC-MS (ESI⁺) m/z 326.3 (M+H)⁺; ¹H NMR (400 MHz, CDCl₃) δ 7.07 (d, J=8.0 Hz, 1H), 6.93-6.85 (m, 2H), 6.48 (t, J=75.2 Hz, 1H), 3.83 (s, 3H), 3.12-3.03 (m, 1H), 2.88 (t, J=3.2 Hz, 1H), 2.54 (d, J=3.2 Hz, 2H), 2.45-2.34 (m, 1H), 2.32-2.22 (m, 4H), 2.22-2.13 (m, 2H), 2.13-2.09 (m, 1H), 2.09-2.00 (m, 2H).

### Step 7-(3aS,7aS)-3a-[4-(difluoromethoxy)-3-methoxy-phenyl]-1-methyl-2,3,4,5,7,7a-hexahydroindol-6-one (341) & (3aR,7aR)-3a-[4-(difluoromethoxy)-3-methoxy-phenyl]-1-methyl-2,3,4,5,7,7a-hexahydroindol-6-one (342)

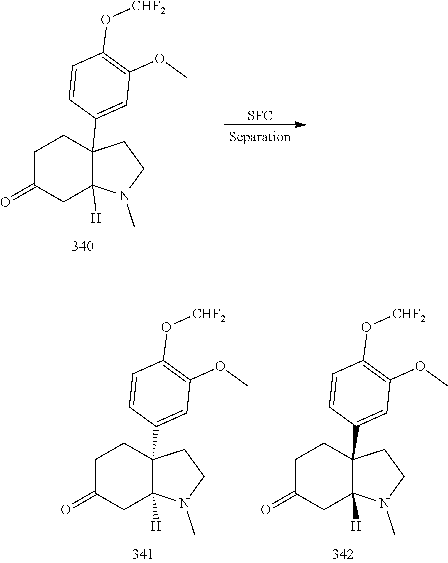

Racemic 340 was separated by SFC (column: Daicel ChiralPak IG (250*30 mm, 10 μm); mobile phase: [CO₂-MeOH]; B %: 20%, isocratic elution mode) to give 341 (1.62 g, 39% yield) as a yellow gum and 342 (1.16 g, 28% yield) as a yellow gum.
341 (retention time: 0.906 min): LC-MS (ESI⁺) m/z 326.0 (M+H)⁺; ¹H NMR (400 MHz, CDCl₃) δ 7.16 (d, J=8.0 Hz, 1H), 7.04-6.90 (m, 2H), 6.80-6.32 (m, 1H), 3.92 (d, J=3.2 Hz, 3H), 3.18 (s, 1H), 2.98 (s, 1H), 2.64 (d, J=2.4 Hz, 2H), 2.47 (dd, J=3.6, 12.4 Hz, 1H), 2.35 (s, 4H), 2.30-2.18 (m, 3H), 2.17-2.08 (m, 2H).
342 (retention time: 1.241 min): LC-MS (ESI⁺) m/z 326.0 (M+H)⁺; ¹H NMR (400 MHz, CDCl₃) δ 7.16 (d, J=8.0 Hz, 1H), 7.03-6.90 (m, 2H), 6.81-6.34 (m, 1H), 3.92 (s, 3H), 3.17 (d, J=6.0 Hz, 1H), 2.99 (s, 1H), 2.64 (s, 2H), 2.54-2.43 (m, 1H), 2.42-2.31 (m, 4H), 2.30-2.21 (m, 2H), 2.20-2.07 (m, 3H).

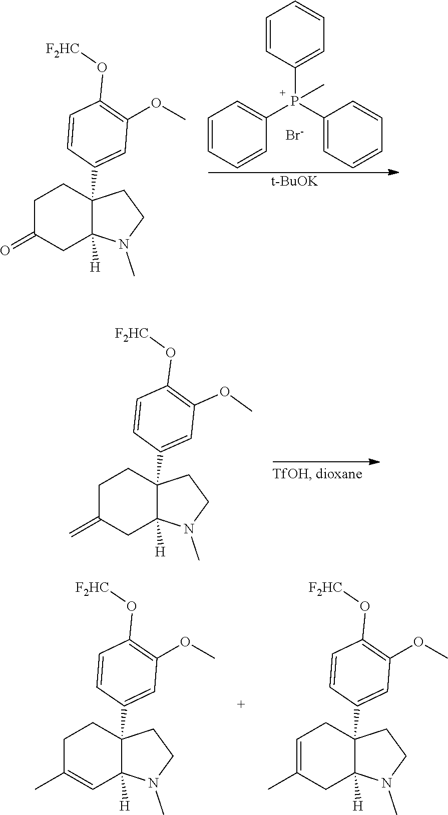

Step 8: Compound 341 is added to a solution of methyl(triphenyl)phosphonium bromide 2.07 mmol) in THF (6.0 mL) is added t-BuOK (1 M in THF, 2.07 mL) at 0° C. over 0.5 hours. 016 (1.04 mmol) in THF (1 mL) is added to the mixture. The reaction mixture was allowed to stir at 25° C. for 16 hr and then poured into water (50 mL). The mixture was extracted with ethyl acetate (100 mL). The organic solutions are dried over sodium sulfate, filtered and concentrated. The crude product is purified by reversed-phase HPLC (0.1% FA condition).

Step 9: The product of Step 8 is added to a solution of 3a-(3,4-dimethoxyphenyl)-1-methyl-6-methylene-2,3,3a,6,7,7a-hexahydro-1H-indole (696 μmol) in dioxane (10 mL) is then added TfOH (1.39 mmol). The mixture was allowed to stir at 80° C. for 2 h and then filtered. The filtrate is concentrated under reduced pressure. The residue is purified by prep-HPLC.

### Example 8: Synthesis of Compounds 230 and 231

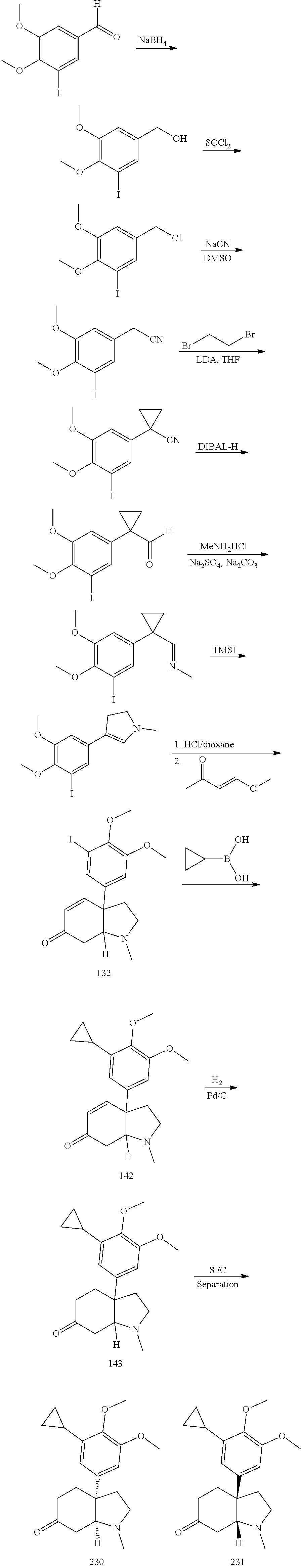

### Step 1-(3-iodo-4,5-dimethoxy-phenyl)methanol

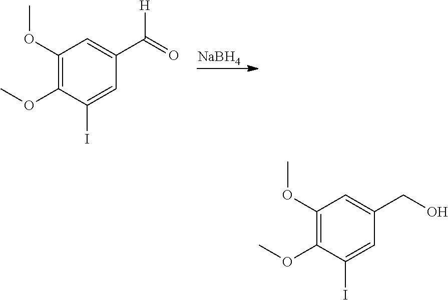

To a solution of 3-iodo-4,5-dimethoxy-benzaldehyde (10 g, 34.2 mmol) in EtOH (100 mL) was added NaBH₄ (1.30 g, 34.2 mmol) in batches at 0° C. The mixture was allowed to stir at 25° C. for 1.5 hours. The mixture was poured into saturated ammonium chloride solution (20 mL) and extracted with ethyl acetate (100 mL). The organic solutions were combined, dried over sodium sulfate, filtered and concentrated in vacuo to give (3-iodo-4,5-dimethoxy-phenyl)methanol (10 g, 89% yield) as a yellow solid. ¹H NMR (400 MHz, CDCl₃) δ 7.24 (s, 1H), 6.83 (s, 1H), 4.52 (s, 2H), 3.76 (d, J=17.6 Hz, 7H).

### Step 2-5-(chloromethyl)-1-iodo-2,3-dimethoxy-benzene

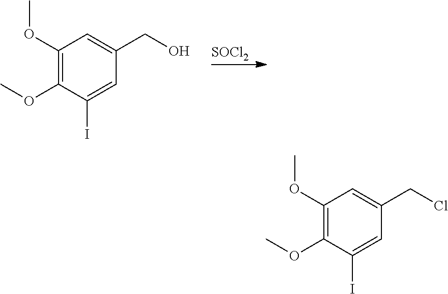

To a solution of (3-iodo-4,5-dimethoxy-phenyl)methanol (10 g, 34.0 mmol) in DCM (100 mL) was added SOCl₂ (16.4 g, 137 mmol, 10 mL). The mixture was allowed to stir at 0° C. for 2 hours. The reaction mixture was concentrated in vacuo, and saturated sodium bicarbonate solution was added to adjust the pH>7. The solution was extracted with ethyl acetate (600 mL). The organic solutions were combined, dried over sodium sulfate, filtered and concentrated in vacuo to give 5-(chloromethyl)-1-iodo-2,3-dimethoxy-benzene (10 g, 84% yield) as a yellow solid. ¹H NMR (400 MHz, CDCl₃) δ 7.29 (d, J=2.0 Hz, 1H), 6.85 (d, J=2.0 Hz, 1H), 4.42 (s, 2H), 3.78 (d, J=17.1 Hz, 6H).

### Step 3-2-(3-iodo-4,5-dimethoxy-phenyl)acetonitrile 2-(3-iodo-4,5-dimethoxy-phenyl)acetonitrile

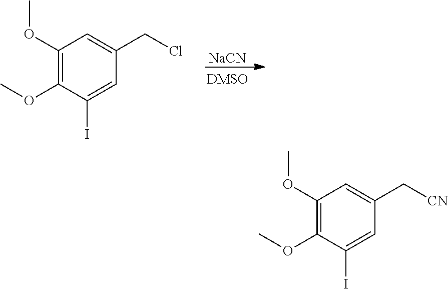

To a solution of 5-(chloromethyl)-1-iodo-2,3-dimethoxy-benzene (10 g, 32.0 mmol) in DMSO (100 mL) was added NaCN (3.14 g, 63.9 mmol). The mixture was allowed to stir at 25° C. for 16 hours. To the mixture was added saturated sodium bicarbonate solution to adjust pH>7. The solution was extracted with ethyl acetate (1000 mL). The organic solutions were combined, washed with brine (500 mL), dried over sodium sulfate, filtered and concentrated in vacuo to give 2-(3-iodo-4,5-dimethoxy-phenyl)acetonitrile 2-(3-iodo-4,5-dimethoxy-phenyl)acetonitrile (9.0 g, 88% yield) as a yellow solid. ¹H NMR (400 MHz, CDCl₃) δ 7.22 (d, J=1.6 Hz, 1H), 6.78 (d, J=1.6 Hz, 1H), 3.81 (s, 3H), 3.76 (s, 3H), 3.60 (s, 2H).

### Step 4-1-(3-iodo-4,5-dimethoxy-phenyl)cyclopropanecarbonitrile

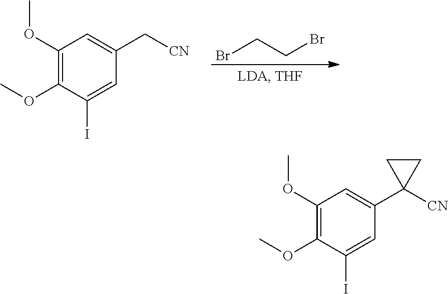

To a solution of 2-(3-iodo-4,5-dimethoxy-phenyl)acetonitrile (9 g, 29.6 mmol, 1 eq.) in THF (90 mL) was added LDA (2 M, 37.1 mL) at 0° C. Next, 1,2-dibromoethane (6.69 g, 35.6 mmol, 2.69 mL) was added to the mixture at 0° C. The mixture was allowed to stir at 25° C. for 2 hours. The mixture was poured into saturated ammonium chloride solution (100 mL) and extracted with ethyl acetate (450 mL). The organic solutions were combined, dried over sodium sulfate, filtered and concentrated in vacuo. The residue was purified by column chromatography (SiO₂, Petroleum ether/Ethyl acetate=1/0 to 1/5 to give 1-(3-iodo-4,5-dimethoxy-phenyl)cyclopropanecarbonitrile (5.8 g, 53% yield) as a yellow solid. ¹H NMR (400 MHz, CDCl₃) δ 7.09 (d, J=2.0 Hz, 1H), 6.86 (d, J=2.0 Hz, 1H), 3.90-3.68 (m, 6H), 1.63 (d, J=2.4 Hz, 2H), 1.30 (d, J=2.4 Hz, 2H).

### Step 5-1-(3-iodo-4,5-dimethoxy-phenyl)cyclopropanecarbaldehyde

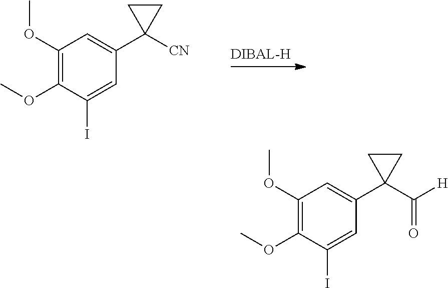 [SMILES: COc1cc(C2(C#N)CC2)cc(I)c1OC ; COc1cc(C2(C=O)CC2)cc(I)c1OC]

To a solution of 1-(3-iodo-4,5-dimethoxy-phenyl)cyclopropanecarbonitrile (5.8 g, 17.6 mmol) in THF (60 mL) was added DIBAL-H (1 M, 26.4 mL). The mixture was allowed to stir at 0° C. for 16 hours. On completion, the mixture was poured into diluted hydrochloric acid (2 M, 20 mL) and extracted with ethyl acetate (60 mL). The organic solutions were combined, dried over sodium sulfate, filtered and concentrated in vacuo to give 1-(3-iodo-4,5-dimethoxy-phenyl)cyclopropanecarbaldehyde (5.8 g, 94% yield) as a yellow solid. ¹H NMR (400 MHz, CDCl₃) δ 9.09 (s, 1H), 7.22-7.18 (m, 1H), 6.76 (d, J=2.0 Hz, 1H), 3.78 (d, J=10.4 Hz, 6H), 1.48 (d, J=2.8 Hz, 2H), 1.34-1.28 (m, 2H)

### Step 6-(E)-1-[1-(3-iodo-4,5-dimethoxy-phenyl)cyclopropyl]-N-methyl-methanimine

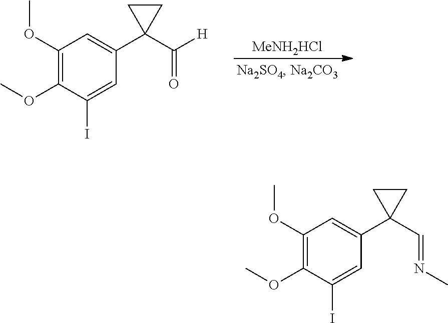

To a solution of 1-(3-iodo-4,5-dimethoxy-phenyl)cyclopropanecarbaldehyde (5.8 g, 17.4 mmol) in DCM (60 mL) was added methanamine hydrochloride (5.90 g, 87.3 mmol), Na₂CO₃ (5.55 g, 52.3 mmol) and Na₂SO₄ (37.2 g, 261 mmol). The mixture was allowed to stir at 25° C. for 16 hours. The reaction mixture was filtered and concentrated in vacuo to give (E)-1-[1-(3-iodo-4,5-dimethoxy-phenyl)cyclopropyl]-N-methyl-methanimine (5.5 g, 85% yield) as a yellow oil. 1H NMR (400 MHz, CDCl₃) δ 7.41 (d, J=1.6 Hz, 1H), 7.22 (d, J=2.0 Hz, 1H), 6.77 (d, J=2.0 Hz, 1H), 3.76 (d, J=13.6 Hz, 6H), 3.17 (d, J=1.6 Hz, 3H), 1.21-1.16 (m, 2H), 1.09-1.02 (m, 2H).

### Step 7-4-(3-iodo-4,5-dimethoxy-phenyl)-1-methyl-2,3-dihydropyrrole

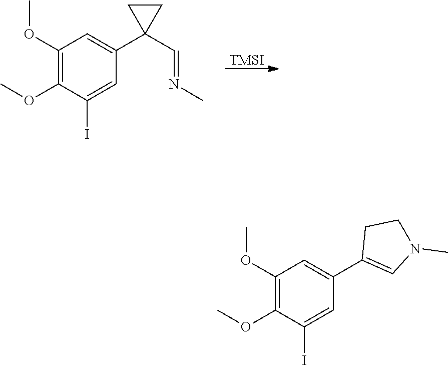

A mixture of (E)-1-[1-(3-iodo-4,5-dimethoxy-phenyl)cyclopropyl]-N-methyl-methanimine (1 g, 2.90 mmol) and TMSI (579 mg, 2.90 mmol, 394 uL) in DMF (10 mL) was degassed and purged with N₂ 3 times, and then the mixture was allowed to stir at 60° C. for 0.5 hours under an atmosphere of N₂. The mixture was poured into water (15 mL) and extracted with ethyl acetate (100 mL). To the aqueous solution was added saturated sodium bicarbonate solution to adjust pH>7, and then this solution was further extracted with ethyl acetate (200 mL). The organic solutions were combined, dried over sodium sulfate, filtered and concentrated in vacuo to give 4-(3-iodo-4,5-dimethoxy-phenyl)-1-methyl-2,3-dihydropyrrole (500 mg, 40% yield) as a yellow oil LC-MS (ESI⁺) m/z 345.9 (M+H)⁺; ¹H NMR (400 MHz, CDCl₃) δ 7.07 (d, J=1.6 Hz, 1H), 6.64 (d, J=1.6 Hz, 1H), 6.30 (s, 1H), 3.77 (s, 3H), 3.72 (s, 3H), 3.10 (t, J=9.2 Hz, 2H), 2.67 (t, J=9.2 Hz, 2H), 2.59 (s, 3H).

### Step 8-(3aR,7aS)-3a-(3-iodo-4,5-dimethoxy-phenyl)-1-methyl-2,3,7,7a-tetrahydroindol-6-one (132)

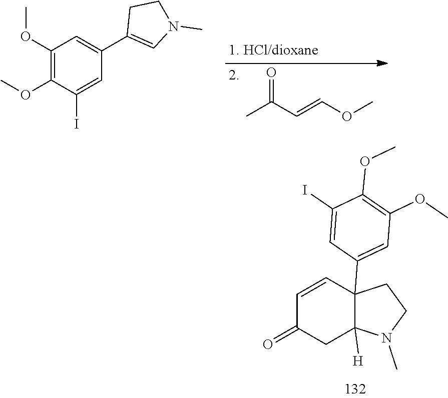

To a solution of 4-(3-iodo-4,5-dimethoxy-phenyl)-1-methyl-2,3-dihydropyrrole (400 mg, 938 μmol) in DCM (4.0 mL) was added HCl/dioxane (4 M, 234 uL) at 25° C. over 10 minutes. After addition, the mixture was filtered and concentrated in vacuo. And then (E)-4-methoxybut-3-en-2-one (112 mg, 1.13 mmol, 113 uL) in ACN (4.0 mL) was added the residue. The mixture was allowed to stir at 60° C. for 2 hours. The reaction mixture was quenched by adding it to a cold saturated aqueous sodium hydroxide solution (30 mL) until pH=8 was achieved. The aqueous solution was extracted with ethyl acetate (100 mL). The organic solutions were combined, dried over anhydrous Na₂SO₄, filtered and concentrated under reduced pressure. The residue was purified by prep-HPLC (FA condition: column: Phenomenex luna C₁₈ 150*25 mm*10 μm; mobile phase: [water (FA)-ACN]; B %: 9%-39%, 10 min) to give 132 (80 mg, 19% yield) as a yellow gum. LC-MS (ESI⁺) m/z 413.9 (M+H)⁺; ¹H NMR (400 MHz, CDCl₃) δ 7.32 (d, J=2.0 Hz, 1H), 6.87 (d, J=2.0 Hz, 1H), 6.71 (dd, J=1.6, 10.0 Hz, 1H), 6.18 (d, J=10.4 Hz, 1H), 3.87 (d, J=11.2 Hz, 6H), 3.38 (dt, J=3.2, 9.2 Hz, 1H), 2.85 (s, 1H), 2.71-2.60 (m, 2H), 2.55 (d, J=4.0 Hz, 1H), 2.50 (dd, J=3.6, 11.1 Hz, 1H), 2.40 (s, 3H), 2.32-2.19 (m, 1H).

### Step 9-(3aR,7aS)-3a-(3-cyclopropyl-4,5-dimethoxy-phenyl)-1-methyl-2,3,7,7a-tetrahydroindol-6-one (142)

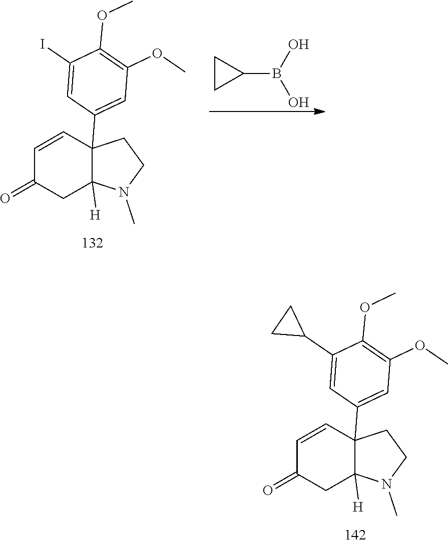

To a solution of 132 (150 mg, 363 μmol) and cyclopropylboronic acid (155 mg, 1.81 mmol) in toluene (3.0 mL) and water (1.0 mL) was added Na₂CO₃ (115 mg, 1.09 mmol) and Pd(PPh₃)₄ (41.9 mg, 36.3 μmol). The mixture was allowed to stir at 90° C. for 12 hours. The mixture was poured to the water (50 mL) and extracted with ethyl acetate (30 mL). The organic solutions were combined, dried over sodium sulfate, filtered and concentrated in vacuo. The crude product was purified by reversed-phase HPLC (0.1% FA condition) to give 142 (70 mg, 57% yield) as a colorless gum. LC-MS (ESI⁺) m/z 328.2 (M+H)⁺. ¹H NMR (400 MHz, CDCl₃) δ 6.77-6.68 (m, 2H), 6.39 (s, 1H), 6.18 (d, J=10.4 Hz, 1H), 3.88 (s, 6H), 3.48-3.32 (m, 1H), 2.94 (br s, 1H), 2.77-2.68 (m, 1H), 2.65 (br s, 1H), 2.59-2.49 (m, 2H), 2.44 (s, 3H), 2.29-2.20 (m, 2H), 1.01 (br d, J=8.4 Hz, 2H), 0.72-0.58 (m, 2H)

### Step 10-3a-(3-cyclopropyl-4,5-dimethoxy-phenyl)-1-methyl-2,3,4,5,7,7a-hexahydroindol-6-one (143)

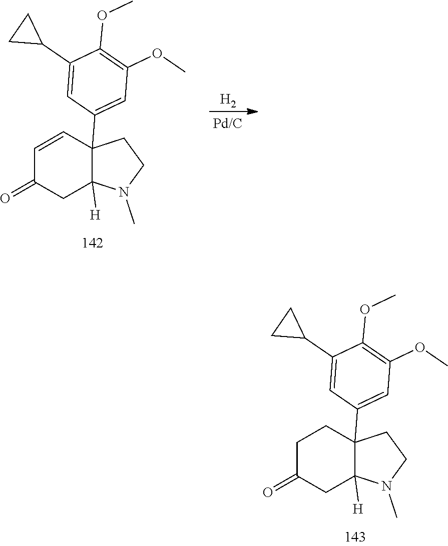 [SMILES: COc1cc(C23C=CC(=O)CC2N(C)CC3)cc(C2CC2)c1OC ; "H2" ; COc1cc(C23CCC(=O)CC2N(C)CC3)cc(C2CC2)c1OC]

To a solution of 142 (200 mg, 610 μmol) in ethyl acetate (1.0 mL) was added Pd/C (10%, 20 mg) under an atmosphere of N₂. The suspension was degassed and purged with H₂ 3 times. The mixture was allowed to stir under H₂ (15 Psi.) at 25° C. for 2 hours. The reaction mixture was filtered and concentrated in vacuo. The residue was purified by prep-HPLC (column: Welch Xtimate C₁₈ 150*25 mm*5 μm; mobile phase: [water (NH₃H₂O)-ACN]; B %: 38%-68%, 8 min) to give the 143 (80 mg, 39% yield) as a yellow solid. LC-MS (ESI⁺) m/z 330.4 (M+H)⁺. ¹H NMR (400 MHz, CDCl₃) δ 6.74 (d, J=2.0 Hz, 1H), 6.42 (d, J=2.0 Hz, 1H), 3.89 (d, J=5.1 Hz, 6H), 3.19-3.10 (m, 1H), 2.93 (br s, 1H), 2.61 (d, J=3.6 Hz, 2H), 2.53-2.37 (m, 2H), 2.33 (s, 3H), 2.31-2.22 (m, 2H), 2.21-2.04 (m, 4H), 1.05-0.96 (m, 2H), 0.72-0.64 (m, 2H)

### Step 11-(3aS,7aS)-3a-(3-cyclopropyl-4,5-dimethoxy-phenyl)-1-methyl-2,3,4,5,7,7a-hexahydroindol-6-one (230) and (3aR,7aR)-3a-(3-cyclopropyl-4,5-dimethoxy-phenyl)-1-methyl-2,3,4,5,7,7a-hexahydroindol-6-one (231)

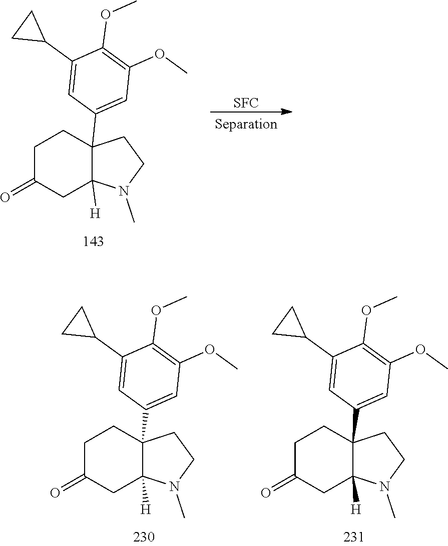

Racemic 143 was separated by SFC (column: DAICEL CHIRALCEL OD (250 mm*30 mm, 10 μm); mobile phase: [Neu-IPA]; B %: 30%-30%, C7.85; 71 min) to give 230 (37.4 mg, 45% yield) as a yellow oil and 231 (40.2 mg, 49% yield) as a yellow oil.
230: LC-MS (ESI⁺) m/z 330.2 (M+H)⁺. ¹H NMR (400 MHz, CDCl₃) δ 6.74 (d, J=2.0 Hz, 1H), 6.41 (d, J=1.6 Hz, 1H), 3.89 (d, J=5.4 Hz, 6H), 3.22-3.09 (m, 1H), 2.94 (br s, 1H), 2.62 (br s, 2H), 2.51-2.40 (m, 1H), 2.34 (s, 4H), 2.29-2.05 (m, 6H), 1.08-0.95 (m, 2H), 0.74-0.62 (m, 2H).
231: LC-MS (ESI⁺) m/z 330.2 (M+H)⁺. ¹H NMR (400 MHz, CDCl₃) δ=6.65 (d, J=2.4 Hz, 1H), 6.32 (d, J=2.0 Hz, 1H), 3.80 (d, J=5.2 Hz, 6H), 3.06 (br t, J=7.2 Hz, 1H), 2.84 (br s, 1H), 2.52 (br d, J=3.6 Hz, 2H), 2.42-2.30 (m, 1H), 2.29-2.22 (m, 4H), 2.21-1.96 (m, 6H), 0.95-0.88 (m, 2H), 0.62-0.55 (m, 2H).

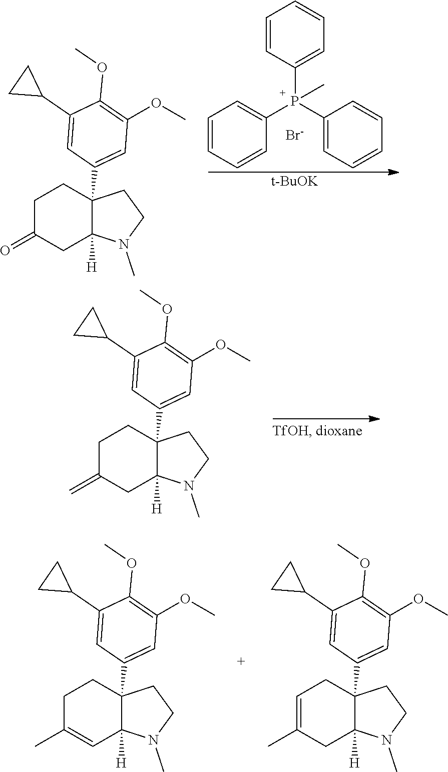

Step 12: Compound 230 is added to a solution of methyl(triphenyl)phosphonium bromide 2.07 mmol) in THF (6.0 mL) is added t-BuOK (1 M in THF, 2.07 mL) at 0° C. over 0.5 hours. 016 (1.04 mmol) in THF (1 mL) is added to the mixture. The reaction mixture was allowed to stir at 25° C. for 16 hr and then poured into water (50 mL). The mixture was extracted with ethyl acetate (100 mL). The organic solutions are dried over sodium sulfate, filtered and concentrated. The crude product is purified by reversed-phase HPLC (0.1% FA condition).

Step 13: The product of Step 12 is added to a solution of 3a-(3,4-dimethoxyphenyl)-1-methyl-6-methylene-2,3,3a,6,7,7a-hexahydro-1H-indole (696 μmol) in dioxane (10 mL) is then added TfOH (1.39 mmol). The mixture was allowed to stir at 80° C. for 2 h and then filtered. The filtrate is concentrated under reduced pressure. The residue is purified by prep-HPLC.

Compounds in the following table could be prepared using synthetic methods similar to those described above:

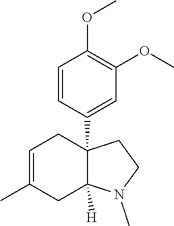

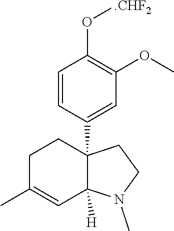

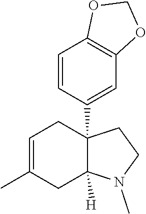 [SMILES: CC1=CC[C@@]2(c3ccc4c(c3)OCO4)CCN(C)[C@H]2C1]

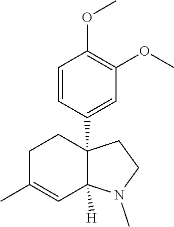

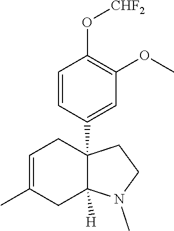

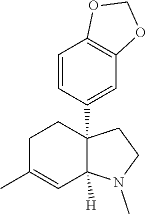 [SMILES: CC1=C[C@@H]2N(C)CC[C@]2(c2ccc3c(c2)OCO3)CC1]

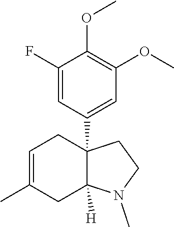

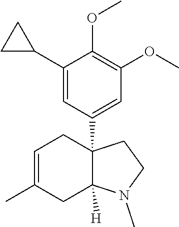 [SMILES: COc1cc([C@@]23CC=C(C)C[C@@H]2N(C)CC3)cc(C2CC2)c1OC]

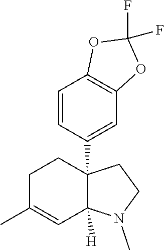 [SMILES: CC1=C[C@@H]2N(C)CC[C@]2(c2ccc3c(c2)OC(F)(F)O3)CC1]

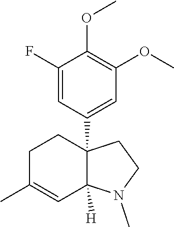

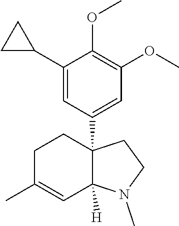 [SMILES: COc1cc([C@@]23CCC(C)=C[C@@H]2N(C)CC3)cc(C2CC2)c1OC]

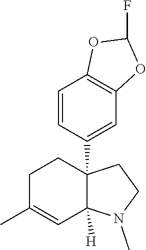 [SMILES: CC1=C[C@@H]2N(C)CC[C@]2(c2ccc3c(c2)OC(F)O3)CC1]

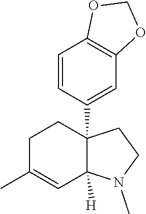 [SMILES: CC1=C[C@@H]2N(C)CC[C@]2(c2ccc3c(c2)OCO3)CC1]

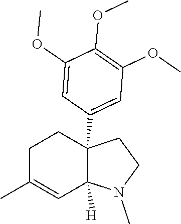

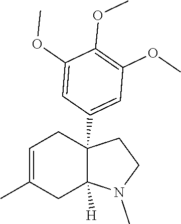

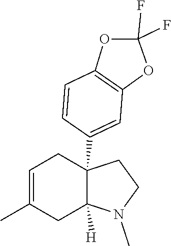 [SMILES: CC1=CC[C@@]2(c3ccc4c(c3)OC(F)(F)O4)CCN(C)[C@H]2C1]

### Example 9: Synthesis of 107, 108 and 109

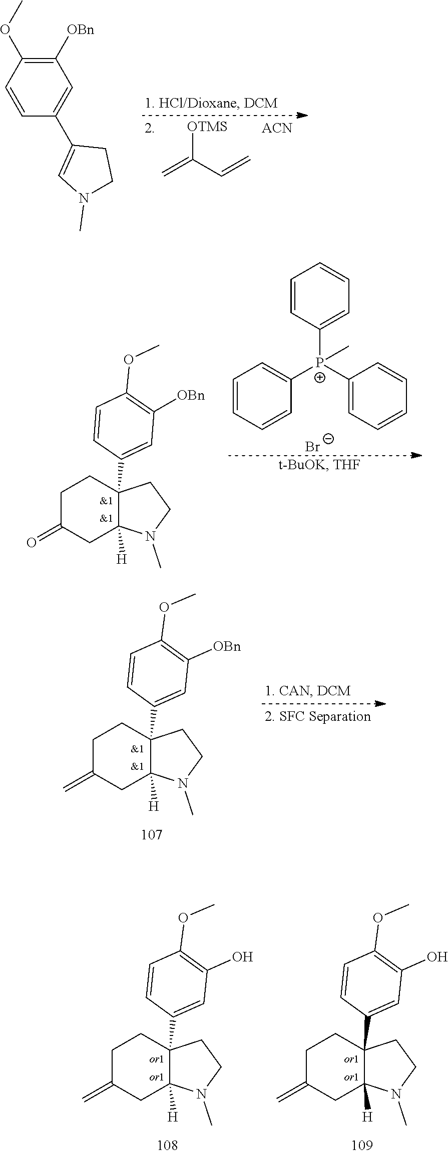

Compound 108 is added to a solution of methyl(triphenyl)phosphonium bromide 2.07 mmol) in THF (6.0 mL) is added t-BuOK (1 M in THF, 2.07 mL) at 0° C. over 0.5 hours. 016 (1.04 mmol) in THF (1 mL) is added to the mixture. The reaction mixture was allowed to stir at 25° C. for 16 hr and then poured into water (50 mL). The mixture was extracted with ethyl acetate (100 mL). The organic solutions are dried over sodium sulfate, filtered and concentrated. The crude product is purified by reversed-phase HPLC (0.1% FA condition).

The product of the previous is added to a solution of 3a-(3,4-dimethoxyphenyl)-1-methyl-6-methylene-2,3,3a,6,7,7a-hexahydro-1H-indole (696 μmol) in dioxane (10 mL) is then added TfOH (1.39 mmol). The mixture was allowed to stir at 80° C. for 2 h and then filtered. The filtrate is concentrated under reduced pressure. The residue is purified by prep-HPLC.

### Example A1: SERT Inhibition Assay

SERT inhibition can be measured using a Neurotransmitter Transportation Fluorescence assay. Briefly, stable 5 HTT cells were prepared in a 384 microwell plate. Compounds were prepared in assay buffer (20 mM HEPES, 0.1% BSA). The compounds were added to the plated cells and incubated for 30 minutes at 37° C. 25 μL of dye solution (Molecular Devices Neurotransmitter Transporter Uptake Assay Kit) is added per well and incubated for 30 minutes at 37° C. The plates are then read on a plate reader.

| | | Relative |
|---|---|---|
| | SERT | SERT |
| Compound | IC₅₀ (nM) | IC₅₀ (nM) |
| 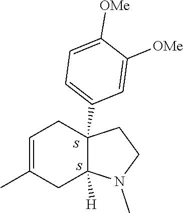 | A | 4.7 |
| 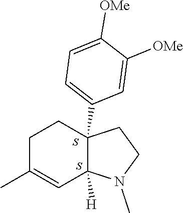 | A | 1.6 |
| 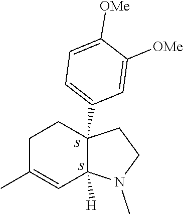 | A | 1.0 |

The results can be provided as follows: A: IC₅₀</=50 nM or lower; B: 50 nM<IC₅₀</=100 nM; C: 100 nM<IC₅₀</=500 nM; D: 500 nM<IC₅₀</=1 micromolar; E: IC₅₀>1 micromolar. The relative SERT IC₅₀ refers to a ratio of the IC₅₀ values for the compounds shown in the table above.

### Example B1: Metabolic Stability of 4 Compounds in Human, Monkey, Dog and Rat Intestinal S9

TABLE B1
Metabolic Stability

| | | Compound ID | | |
|---|---|---|---|---|
| Assay | Unit | 001 | 243 | 242 |
| Human Hepatocyte | Stability Improvement | 1 | 3.5X | 2.9X |
| Stability (CLint) | Relative to Compound 001 | | | |
| Human Intestinal S9 | Stability Improvement | 1 | 11X | 10X |
| (CLint) | Relative to Compound 001 | | | |
| SERT | IC₅₀ (nM) | A | A | A |

### Hepatocyte Stability:

10 mM stock solutions of test compounds and positive control were prepared in DMSO. Thawing medium and supplement incubation medium (serum-free) were placed in a 37° C. water bath for at least 15 minutes prior to use. Stock solutions were diluted to 100 μM by combining 198 μL acetonitrile and 2 μL of 10 mM stock solution. Verapamil was used as positive control in the assay. Vials of cryopreserved pooled human mixed gender hepatocytes were removed from storage, ensured that vials remain at cryogenic temperatures. The pressure was removed by loosening and re-tightening the cap. The vials were thawed in a 37° C. water bath with gently shaking. Vials remained in water bath until all ice crystals had dissolved and were no longer visible. Vials were sprayed with 70% ethanol before being transferred to a biosafety cabinet. And then the contents were poured into the 50 mL thawing medium conical tube. Vials were centrifuged at 100 g for 10 minutes at room temperature. Thawing medium was aspirated and hepatocytes were re-suspended with serum-free incubation medium to yield ˜1.5×10⁶ cells/mL. Cell viability and density were counted using Cellometer® Vision, and then cells were diluted with serum-free incubation medium to a working cell density of 0.5×10⁶ viable cells/mL. Aliquots of 198 μL hepatocytes were dispensed into each well of a 96-well non-coated plate. The plate was placed in the incubator on an orbital shaker at 500 rpm for approximately 10 minutes. Aliquots of 2 μL of the 100 μM test compounds or verapamil were added into respective wells of the non-coated 96-well plate to start the reaction. This assay was performed in duplicate. The plate was incubated in the incubator on an orbital shaker at 500 rpm for the designed time points. 25 μL of contents were transferred and mixed with 5 volumes (125 L) of cold acetonitrile with IS (100 nM alprazolam, 100 nM labetalol and 100 nM tolbutamide) to terminate the reaction at time points of 0, 15, 30, 60, 90 and 120 minutes. Samples were centrifuges for 30 minutes at 3,220 g. Then transfer 100 μL of the supernatant to new 96-well plates for analysis. Add 100 μL of distilled water to each sample and mix for analysis by LC-MS/MS. Peak areas were determined from extracted ion chromatograms. Determine the in vitro half-life (t_{1/2}) of parent compound by regression analysis of the percent parent disappearance vs. time curve. The in vitro half-life (in vitro t_{1/2}) was determined from the slope value: in vitro t_{1/2}=0.693/k. b. Conversion of the in vitro t_{1/2} (in min) into the in vitro intrinsic clearance (in vitro CLᵢₙₜ, in uL/min/10⁶ cells) is done using the following equation:

in vitro CLᵢₙₜ=kV/N
V=incubation volume (0.2 mL);
N=number of hepatocytes per well (0.1×10⁶ cells).
Hepatocyte Stability Data in table B1 expressed as CLint Times (X) improvement relative to compound 001.

### Intestinal Stability:

A master solution was prepared according to the Table B2 below.

TABLE B2
Preparation of Master Solution

| | Stock | | | | Final | |
|---|---|---|---|---|---|---|
| Reagent | Concentration | | Volume | | Concentration | |
| Phosphate buffer | 200 | mM | 125 | μL | 100 | mM |
| Ultra-pure H₂O | — | | 10 | μL | — | |
| MgCl₂ solution | 50 | mM | 25 | μL | 5 | mM |
| Intestinal S9 fractions | 4 | mg/mL | 62.5 | μL | 1 | mg/mL |

Add 222.5 μL of master solution and 25 μL of 10 mM NADPH solution to the incubation plates. Pre-warm for 10 min. The reaction was started with the addition of 2.5 μL of 100 μM control compound or test compound solutions. Verapamil, Midazolam and Testosterone were used as positive control in this study. The final concentration of control compounds and test compounds was 1 μM. Aliquots of 25 μL were taken from the reaction solution at 0.5, 5, 15, 30, 45 and 60 minutes. The reaction was stopped by the addition of 5 volumes of cold acetonitrile with IS (100 nM alprazolam, 200 nM caffeine and 100 nM tolbutamide). Samples were centrifuged at 3, 220 g for 30 minutes. Aliquot of 100 μL of the supernatant was mixed with 100 μL of ultra-pure H₂O and then used for LC-MS/MS analysis.

Data analysis was performed as follows. All calculations were carried out using Microsoft Excel. Peak areas were determined from extracted ion chromatograms. The slope value, k, was determined by linear regression of the natural logarithm of the remaining percentage of the parent drug vs. incubation time curve. The in vitro half-life (in vitro t_{1/2}) was determined from the slope value:

$$in vitro {t}_{1 / 2} = \text{?} ( 0.693 / k )$$

$$\text{?} \text{indicates text missing or illegible when filed}$$

Conversion of the in vitro t_{1/2} (min) into the in vitro intrinsic clearance (in vitro CLᵢₙₜ, in uL/min/mg protein) was done using the following equation (mean of duplicate determinations):

$$in vitro C \text{?} = ( \frac{0.693}{( {t}_{1 / 2} )} ) \text{?} ( \frac{volume of incubation ( μL )}{amount of proteins ( mg )} )$$

$$\text{?} \text{indicates text missing or illegible when filed}$$

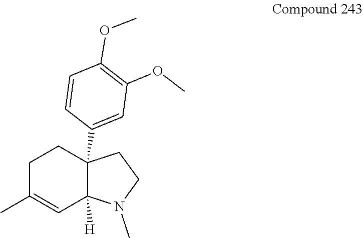

### Example 3

The metabolic stability of Compound 243 was tested in human, monkey, dog and rat (2 replicate measurement). For comparison, verapamil was tested in human and monkey (2 replicates), midazolam was tested in dog (2 replicates), and testosterone was tested in rat (2 replicates).

In human metabolic stability testing, the median half-life of compound 243 was greater than 24 hours and 33 times greater than verapamil, with a median CLint (micrograms/min/mg protein) that was more than 23 times greater for verapamil than Compound 243.

In dog metabolic stability testing, the median half-life of Compound 243 was more than 168 times greater than the half-life of midazolam, with a median CLint (micrograms/min/mg protein) that was more than 164 times greater for midazolam than Compound 243 in dogs.

In rat metabolic stability testing, the median half-life of Compound 243 was about 30 times greater than the half-life of testosterone, with a median CLint (micrograms/min/mg protein) that was about 30 times greater for testosterone than Compound 243 in dogs.

1-21. (canceled)

22. A compound of formula (I), or a pharmaceutically acceptable salt thereof: 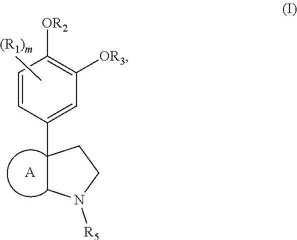
wherein
ring A is 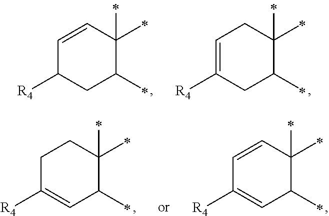
wherein * denotes the attachment points of ring A to the compound of formula (I), and wherein
each R₁ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, —C(O) NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O) NR^{a}R^{b}, —CN, nitro, or —P(O)OR^{a}OR^{b};
m is 0, 1, 2, or 3;
each of R₂ and R₃ is independently H, alkyl, alkenyl, alkynyl, cycloalkyl, heterocycloalkyl, or aryl; wherein each hydrogen atom in alkyl cycloalkyl, alkenyl, alkynyl, and aryl is optionally substituted by halo, deuterium, cycloalkyl, aryl, or OR^{a}; or
R^{a} and R^{b} are independently H, alkyl, alkenyl, cycloalkyl, heterocycloalkyl, aryl, or heteroaryl; or if an instance of R₁ is —NR^{a}R^{b}, then R^{a} and R^{b} may combine with the nitrogen atom to which they are attached to form heterocycloalkyl or heteroaryl;
R₄ is H, C₁₋₄ alkyl, or C₁₋₆ haloalkyl; and
R₅ is C₁₋₄ alkyl optionally substituted with C₃₋₆ cycloalkyl, 3- to 6-membered heterocycle, phenyl or 5- to 6-membered heteroaryl, wherein the alkyl, cycloalkyl, heterocycle, phenyl or heteroaryl are each independently optionally substituted with one or more deuterium, C₁₋₄ alkoxy, C₁₋₄ haloalkyl, halo, cyano, amino, carboxy, acetyl, or amide.

23. The compound of claim 22, wherein R₄ is methyl.

24. The compound of claim 23, wherein R₅ is methyl.

25. The compound of claim 24, wherein each R₁ is independently halo, C₁₋₄ alkyl, C₁₋₄ alkenyl, C₁₋₄ alkynyl, C₃₋₆ cycloalkyl, —OR^{a}, —NR^{a}R^{b}, —CHO, —C(O)R^{a}, —CO₂R^{a}, —C(O) NR^{a}R^{b}, —CN, nitro, or —P(O)OR^{a}OR^{b}; wherein each hydrogen atom in alkyl, alkenyl, alkynyl, or cycloalkyl is optionally substituted by halo, C₁₋₄ alkyl, C₁₋₄ alkanol, —OR^{a}, —NR^{a}R^{b}, —CHO, —C(O)R^{a}, —CO₂R^{a}, —C(O)NR^{a}R^{b}, —CN, nitro, or —P(O)OR^{a}OR^{b}; and R^{a} and R^{b} are each independently H, or C₁₋₄ alkyl.

26. The compound of claim 25, wherein each R₁ is independently halo, C₁₋₄ alkyl, C₁₋₄ haloalkyl, C₁₋₄ alkoxy, acetyl, cyano, C₃₋₆ cycloalkyl, or C(O)NR^{a}R^{b}; R^{a} and R^{b} are each C₁₋₄ alkyl.

27. The compound of claim 26, wherein
a. R₂ and R₃ are each independently H, C₁₋₄ haloalkyl, C₁₋₄ alkoxy, C₃₋₆ cycloalkyl or C₁₋₄ alkyl optionally substituted with C₃₋₆ cycloalkyl, phenyl, C₂₋₄ alkenyl or C₂₋₄ alkynyl; or
b. R₂ and R₃ together with the atoms to which they are attached combine to form a 5- to 6-membered heterocyclyl, wherein each hydrogen atom in the heterocyclyl is optionally substituted by halo or OR^{a}; and R^{a} is C₁₋₄ alkyl, C₁₋₄ haloalkyl or C₁₋₄ alkoxy.

28. The compound of claim 27, wherein one of R₂ and R₃ is not H.

29. The compound of claim 28, wherein R₂ and R₃ are each independently H, C₁₋₄ haloalkyl, —(CH₂)—(C₃₋₆ cycloalkyl), —(CH₂)—(C₁₋₄ alkoxy), C₃₋₆ cycloalkyl, or benzyl.

30. The compound of claim 29, wherein ring A is

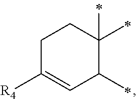

31. The compound of claim 30, wherein the compound is a compound of Formula (II-C), 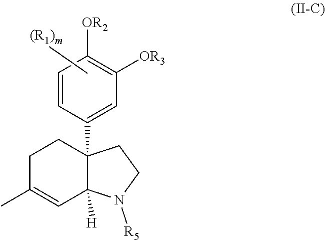
or a pharmaceutically acceptable salt thereof.

32. The compound of claim 31, wherein R₂ and R₃ are each independently methyl.

33. The compound of claim 32, wherein m is 0 or 1.

34. The compound of claim 22, wherein the compound is 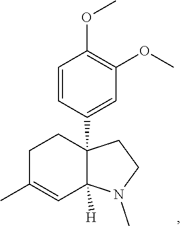
or a pharmaceutically acceptable salt thereof.

35. The compound of claim 22, wherein the compound is 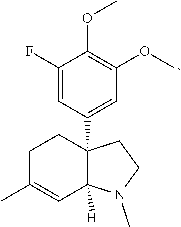
or a pharmaceutically acceptable salt thereof.

36. The compound of claim 22, wherein the compound is 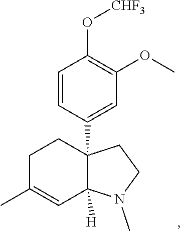
or a pharmaceutically acceptable salt thereof.

37. The compound of claim 22, wherein the compound is 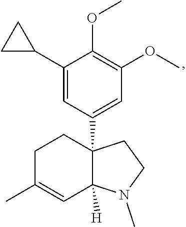 [SMILES: COc1cc([C@@]23CCC(C)=C[C@@H]2N(C)CC3)cc(C2CC2)c1OC]
or a pharmaceutically acceptable salt thereof.

38. The compound of claim 27, wherein R₂ and R₃ together with the atoms to which they are attached combine to form a 5- to 6-membered heterocyclyl, wherein each hydrogen atom in the heterocyclyl is optionally substituted by halo or OR^{a}; and R^{a} is C₁₋₄ alkyl, or C₁₋₄ haloalkyl.

39. The compound of claim 37, wherein R^{a} is methyl and the halo is fluoro.

40. A compound of formula (I), or a pharmaceutically acceptable salt thereof: 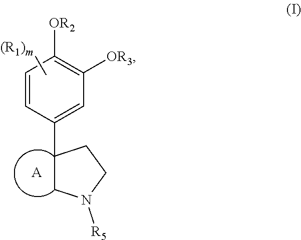
wherein
ring A is 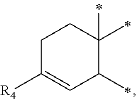
wherein * denotes the attachment points of ring A to the compound of formula (I); and wherein
each R₁ is independently deuterium, halo, alkyl, alkenyl, alkynyl, cycloalkyl, cycloalkenyl, heterocyclyl, aryl, heteroaryl, —OR^{a}, —NR^{a}R^{b}, —CHO, —C(O)R^{a}, —CO₂R^{a}, C(O)NR^{a}R^{b}, —CN, nitro, or —P(O)OR^{a}OR^{b}; wherein each hydrogen atom in alkyl, alkenyl, alkynyl, cycloalkyl, cycloalkenyl, heterocyclyl, aryl, and heteroaryl is optionally substituted by halo, alkyl, alkanol, aryl, —OR^{a}, —NR^{a}R^{b}, —CHO, —C(O)R^{a}, —CO₂R^{a}, —C(O) NR^{a}R^{b}, —CN, nitro, or —P(O)OR^{a}OR^{b};
m is 0, 1, 2, or 3;
each of R₂ and R₃ is independently H, alkyl, alkenyl, alkynyl, cycloalkyl, heterocycloalkyl, or aryl; wherein each hydrogen atom in alkyl cycloalkyl, alkenyl, alkynyl, and aryl is optionally substituted by halo, deuterium, cycloalkyl, aryl, or OR^{a}; or
R^{a} and R^{b} are independently H, alkyl, alkenyl, cycloalkyl, heterocycloalkyl, aryl, or heteroaryl; or if an instance of R₁ is —NR^{a}R^{b}, then R^{a} and R^{b} may combine with the nitrogen atom to which they are attached to form heterocycloalkyl or heteroaryl;
R₄ is H, C₁₋₄ alkyl, or C₁₋₆ haloalkyl; and
R₅ is methyl.

41. A pharmaceutical composition, comprising a compound of claim 22 or a pharmaceutically acceptable salt thereof; and a pharmaceutically acceptable excipient or carrier.

42. A method of treating a central nervous system condition, comprising administering to a subject in need thereof an effective amount of a compound of claim 22 or a pharmaceutically acceptable salt thereof.
